SEQUENCE LISTING (88 sequences)

SEQ ID NO: 1 - DNA, 5483 bp, Artificial Sequence
  SYNTHESIZED
caggacacaaggcctgttactagcactcacatggaacaaatggcccagatctggccgcactcgaaaacgggccctctagactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccctagcgtttccggcgacggtgctagcgtcgaccagtggatcctggaggcttgctgaaggctgtatgctgtacttcagctcctttgaggaagttttggccactgactgacttcctcaagagctgaagta

SEQ ID NO: 2 - DNA, 1197 bp, Mus musculus
atggccaagctcattcttgtcacaggtctggcaattcttctgaacgtacagctgggatcttcctaccagctgatgtgctactataccagttgggctaaggacaggccaatagaagggagtttcaaacctggtaatattgacccctgcctgtgtactcacctgatctatgcctttgctggaatgcagaataatgagatcacttacacacatgagcaagacttgcgtgactatgaagcattgaatggtctgaaagacaagaacactgagctaaaaactctcctggccattggaggatggaagtttggacctgccccgttcagtgccatggtctctactcctcagaaccgtcagatattcattcagtcagttatcagattccttcgtcaatataactttgatggcctcaacctggactggcagtaccctgggtctcgaggaagccctcctaaggacaaacatctcttcagtgttctggtgaaggaaatgcgtaaagcttttgaggaagaatctgtggagaaagacattccaaggctgctactcacttccacaggagcaggaatcattgacgtaatcaagtctgggtacaagatccctgaactgtctcagtctcttgactatattcaggtcatgacatatgatctccatgatcctaaggatggctacactggagaaaatagtcccctctataaatctccatatgacattggaaagagtgctgatctcaatgtggattcaatcatttcctactggaaggaccatggagcagcttctgagaagctcattgtgggatttccagcatatgggcatacctttatcctgagtgacccttctaagactggaattggtgcccctacaattagtactggcccaccaggaaagtacacagatgaatcaggactcctggcttactatgaggtttgtacatttctgaatgaaggagccactgaggtctgggatgccccccaggaagtaccctatgcctatcagggtaatgagtgggttggttatgacaatgtcaggagcttcaagttgaaggctcagtggctcaaggacaacaatttaggaggtgccgtggtctggcccctggacatggatgacttcagtggttctttctgtcaccagagacatttccctctgacatctactttaaagggagatctcaatatacacagtgcaagttgcaagggcccttattga

SEQ ID NO: 3 - DNA, 21 bp, Mus musculus
accagctgatgtgctactata

SEQ ID NO: 4 - DNA, 64 bp, Artificial Sequence
  SYNTHESIZED
tgctgtatagtagcacatcagctggtgttttggccactgactgacaccagctggtgctactata

SEQ ID NO: 5 - DNA, 21 bp, Mus musculus
cctgtgtactcacctgatcta

SEQ ID NO: 6 - DNA, 64 bp, Artificial Sequence
  SYNTHESIZED
tgctgtagatcaggtgagtacacagggttttggccactgactgaccctgtgtacacctgatcta

SEQ ID NO: 7 - DNA, 21 bp, Mus musculus
caagacttgcgtgactatgaa

SEQ ID NO: 8 - DNA, 21 bp, Mus musculus
tctcttcagtgttctggtgaa

SEQ ID NO: 9 - DNA, 21 bp, Mus musculus
agcaggaatcattgacgtaat

SEQ ID NO: 10 - DNA, 21 bp, Mus musculus
tatcctgagtgacccttctaa

SEQ ID NO: 11 - DNA, 64 bp, Artificial Sequence
  SYNTHESIZED
tgctgttagaagggtcactcaggatagttttggccactgactgactatcctgagacccttctaa

SEQ ID NO: 12 - DNA, 21 bp, Mus musculus
tgaatcaggactcctggctta

SEQ ID NO: 13 - DNA, 21 bp, Mus musculus
atgtcaggagcttcaagttga

SEQ ID NO: 14 - DNA, 64 bp, Artificial Sequence
  SYNTHESIZED
tgctgtcaacttgaagctcctgacatgttttggccactgactgacatgtcaggcttcaagttga

SEQ ID NO: 15 - DNA, 21 bp, Mus musculus
ggctcaaggacaacaatttag

SEQ ID NO: 16 - DNA, 21 bp, Mus musculus
ttccctctgacatctacttta

SEQ ID NO: 17 - DNA, 1395 bp, Mus musculus
atggtgcagtccctggcctgggcaggtgtgatgactctgctgatggtccagtggggctctgctgcaaaactggtctgctacctcaccaactggtcccagtaccggacggaggcagttcggttctttcccagggatgtggatcccaacctgtgtacccacgtcatctttgcttttgctggaatggacaaccatcagctcagcactgtggagcacaatgacgaacttctctaccaggagctgaacagcctaaagactaagaaccccaagctcaagaccctgttagccgttggaggctggacctttggtacccagaagttcacagacatggtggccaccgccagcaaccggcagacctttgtgaagtcagccctaagtttcctgcgcactcaaggttttgatggccttgaccttgactgggagttcccaggtggacgtgggagccccacagtagacaaagagagattcacagccctgatacaggacttggccaaagccttccaggaggaagcccagtcctcagggaaggaacgcctccttctgactgcagctgtaccgagtgatcgaggcctggtggatgctggctacgaggtggacaagattgcccagagcttggatttcatcaaccttatggcctacgacttccacagctccttggaaaagaccacagggcataacagccccctctacaaaaggcaaggagaaagtggggcagccgctgagcaaaacgtggatgctgctgtgacgctctggctgcagaaggggaccccagccagcaaactgatccttggcatgcctacctatggacgctctttcaccttggcctcctcgtcagacaatggagttggggccccagccacagggcctggtgccccaggcccctatacgaaggacaaaggggtcctggcttactatgaggcctgctcctggaaggaaagacacagaatcgaggaccagaaggtgccttacgccttccaggacaaccagtgggtgagctttgacgacgtggaaagcttcaaagccaaggctgcctacctgaaacagaaggggctgggaggagccatggtctgggtcctggacttggatgacttcaagggttccttctgcaaccagggcccgtaccctctcatccggacactacggcaggaactaaatcttccatccgagactccaaggagcccagaacagataatacctgagccacgcccatcttctatgccagagcagggacccagcccagggctagataacttctgccaaggcaaagctgatggggtctaccccaaccctggagacgagtccacttactacaactgtggaggagggcggctgttccagcagagctgtcctccaggcctggtgtttagagcctcttgcaaatgttgtacctggagctaa

SEQ ID NO: 18 - DNA, 21 bp, Mus musculus
gcacaatgacgaacttctcta

SEQ ID NO: 19 - DNA, 21 bp, Mus musculus
acttctctaccaggagctgaa

SEQ ID NO: 20 - DNA, 64 bp, Artificial Sequence
  SYNTHESIZED
tgctgttcagctcctggtagagaagtgttttggccactgactgacacttctctcaggagctgaa

SEQ ID NO: 21 - DNA, 21 bp, Mus musculus
cctttgtgaagtcagccctaa

SEQ ID NO: 22 - DNA, 21 bp, Mus musculus
tttgtgaagtcagccctaagt

SEQ ID NO: 23 - DNA, 64 bp, Artificial Sequence
  SYNTHESIZED
tgctgacttagggctgacttcacaaagttttggccactgactgactttgtgaacagccctaagt

SEQ ID NO: 24 - DNA, 21 bp, Artificial Sequence
  SYNTHESIZED
cctaagtttcctgcgcactca

SEQ ID NO: 25 - DNA, 64 bp, Artificial Sequence
  SYTHESIZED
tgctgtacagctgcagtcagaaggaggttttggccactgactgacctccttctctgcagctgta

SEQ ID NO: 26 - DNA, 21 bp, Mus musculus
gagagattcacagccctgata

SEQ ID NO: 27 - DNA, 21 bp, Mus musculus
ctccttctgactgcagctgta

SEQ ID NO: 28 - DNA, 21 bp, Mus musculus
gcttggatttcatcaacctta

SEQ ID NO: 29 - DNA, 64 bp, Artificial Sequence
  SYNTHESIZED
tgctgtaaggttgatgaaatccaagcgttttggccactgactgacgcttggatcatcaacctta

SEQ ID NO: 30 - DNA, 21 bp, Mus musculus
tgagctttgacgacgtggaaa

SEQ ID NO: 31 - DNA, 64 bp, Artificial Sequence
  SYNTHESIZED
tgctgtttccacgtcgtcaaagctcagttttggccactgactgactgagctttcgacgtggaaa

SEQ ID NO: 32 - DNA, 21 bp, Mus musculus
gtgtttagagcctcttgcaaa

SEQ ID NO: 33 - DNA, 1395 bp, Rattus rattus
atggtgcagtccctggccttggcaggtgtgatgactctgctgatgatccagtggggctctgctgcaaaactgttctgctacttcaccaactgggcccagtaccggtccggggcagctcgattcctacctagggatgtggatcccaacctgtgtacccatgtcatctatgcctttgctggaatgaacaaccaccagatcagcactgtagagcccaatgacgagcttttctaccaagagctgaacagcctaaagaagaggaaccccaagctcaagaccctgttagccgtcgggggctggagctttggtacccagaagttcacagacatggtggccacagccagcacccggcagacctttgtcaactcagctctctcgttcctgcgcactcatggttttgacggccttgaccttgactgggaatacccaggaagccgagggagcccagcagtagacaaagagagattcacagcgctgatacaggatttggccaaagccttccaggaggaagcccgggcctcagggaaaagtcgcctccttctgactgcagctgtaccaactggtcgaggccatgtggatgctggttatgaggtggacaagattgctcagagcttggatttcatcaaccttatggcctacgacttccacagctcctgggacaagaccacagggcacaacagccccctctacaaaaggcaaggagagactgggaaggatgctgaaaaaaatgtggatgctgctgtgacgctctggctgaagaaggggacccctgccagcaaactgatgcttggcatgcccgcctacggacgctccttcaccctggcctcctcatcagacagtggagttggggccccagccacaggacctggggccccaggcccctatactaaggaaaaggggatactggtttactttgaggtctgctcctggaagggaaaacagagaatcgaggaccagaaggtgccctatgtctcccagggcaaccaatgggtgggatttgatgacagggaaagcttcagagccaaggctgcctatgtgaaacagaagggactaggaggggccatggtctggatcctggatggggatgacttcaaaggttccttctgcaacgagggccagtaccctctcatccggacactacaccaggagctgagcctttcatccgggcctccaagaagcccagaacaggaagtacctgggccacaccagccttctgagcccgagcagggccccagcccagggctagataacttctgccaaggcaaagctgatgggctctaccccaaccctacagaaaagtccagtttctacagctgtggaggggggcgactgttccagcacagctgccccccaggcctggtgtttatcgactcttgcaaatgctgtacctggggttga

SEQ ID NO: 34 - DNA, 21 bp, Artificial Sequence
  SYNTHESIZED
aaccaccagatcagcactgta

SEQ ID NO: 35 - DNA, 64 bp, Artificial Sequence
  SYNTHESIZED
tgctgtacagtgctgatctggtggttgttttggccactgactgacaaccaccatcagcactgta

SEQ ID NO: 36 - DNA, 21 bp, Rattus rattus
gctttggtacccagaagttca

SEQ ID NO: 37 - DNA, 21 bp, Rattus rattus
gcccagcagtagacaaagaga

SEQ ID NO: 38 - DNA, 64 bp, Artificial Sequence
  SYNTHESIZED
tgctgtctctttgtctactgctgggcgttttggccactgactgacgcccagcaagacaaagaga

SEQ ID NO: 39 - DNA, 21 bp, Rattus rattus
ctccttctgactgcagctgta

SEQ ID NO: 40 - DNA, 64 bp, Artificial Sequence; identical to SEQ ID NO: 25
  SYNTHESIZED

SEQ ID NO: 41 - DNA, 21 bp, Rattus rattus
ggccatgtggatgctggttat

SEQ ID NO: 42 - DNA, 21 bp, Rattus rattus
agattgctcagagcttggatt

SEQ ID NO: 43 - DNA, 64 bp, Artificial Sequence
  SYNTHESIZED
tgctgaatccaagctctgagcaatctgttttggccactgactgacagattgctgagcttggatt

SEQ ID NO: 44 - DNA, 21 bp, Rattus rattus
aaccaatgggtgggatttgat

SEQ ID NO: 45 - DNA, 21 bp, Rattus rattus
tgggatttgatgacagggaaa

SEQ ID NO: 46 - DNA, 64 bp, Artificial Sequence
  SYNTHESIZED
tgctgtttccctgtcatcaaatcccagttttggccactgactgactgggattttgacagggaaa

SEQ ID NO: 47 - DNA, 21 bp, Rattus rattus
acttcaaaggttccttctgca

SEQ ID NO: 48 - DNA, 20 bp, Rattus rattus
tggtgtttatcgactcttgc

SEQ ID NO: 49 - DNA, 1146 bp, Mus musculus
atgggcatgagggcggcactgacaggctttgcggtcctgatgctgctccagagctgctctgcgtacaagctggtctgctacttcaccagctggtcccagtaccgggaaggcgttggaagcttcttaccagacgccatccaacctttcctgtgcacccacatcatctacagctttgccaacatcagcagcgacaacatgcttagcacatgggagtggaatgacgagtcgaactatgacaagctgaataaactgaagaccagaaacaccaacctgaagaccctcctgtctgttggagggtggaaatttggcgaaaaaagattttccgagattgcctccaacactgagagacgcactgctttcgtccggtcggtagccccgttcctgcgttcttatggctttgatgggctggatctcgcctggctctaccctcgcttaagagacaagcagtatttctccaccctgatcaaggaactgaatgcggaattcacaaaggaggtccagccaggcagagagaaactcctgctcagcgcagctttgtcagcaggaaaggtggccattgacactggctatgacatcgcccagatagcccaacacctggattttatcaatctcatgacctacgatttccatggagtctggcgccaaatcacaggccaccacagccccctcttccaaggccagaaggacactaggtttgacagatacagcaatgtgaactatgccgtgcagtacatgatacgtctgggagcccaggccagcaagctactgatgggcatccccacctttgggaagagcttcactctggcatcttctgaaaatcagttgggagctccaatctcaggggaaggattaccaggccggttcaccaaggaggcagggaccctggcctactacgagatatgcgacttcctcaaaggagctgaagtacatcgactctccaacgagaaggttcccttcgctaccaagggcaaccagtgggtggggtatgaggacaaggagagtgtcaaaaacaaggttgggttcctgaaggagaagaagctggcaggagccatggtgtgggcactggatttggatgatttccagggcacctgtcagccgaaggaattcttcccgctcaccaacgccatcaaggatgccctggcttag

SEQ ID NO: 50 - DNA, 21 bp, Mus musculus
ctttcctgtgcacccacatca

SEQ ID NO: 51 - DNA, 64 bp, Artificial Sequence
  SYNTHESIZED
tgctgtgatgtgggtgcacaggaaaggttttggccactgactgacctttcctgcacccacatca

SEQ ID NO: 52 - DNA, 21 bp, Mus musculus
cgaactatgacaagctgaata

SEQ ID NO: 53 - DNA, 21 bp, Mus musculus
cactgagagacgcactgcttt

SEQ ID NO: 54 - DNA, 21 bp, Mus musculus
gcttaagagacaagcagtatt

SEQ ID NO: 55 - DNA, 21 bp, Mus musculus
gaactgaatgcggaattcaca

SEQ ID NO: 56 - DNA, 64 bp, Artificial Sequence
  SYNTHESIZED
tgctgtgtgaattccgcattcagttcgttttggccactgactgacgaactgaacggaattcaca

SEQ ID NO: 57 - DNA, 21 bp, Mus musculus
gcagctttgtcagcaggaaag

SEQ ID NO: 58 - DNA, 21 bp, Mus musculus
ctaggtttgacagatacagca

SEQ ID NO: 59 - DNA, 21 bp, Mus musculus
ttcctcaaaggagctgaagta

SEQ ID NO: 60 - DNA, 64 bp, Artificial Sequence
  SYNTHESIZED
tgctgtacttcagctcctttgaggaagttttggccactgactgacttcctcaagagctgaagta

SEQ ID NO: 61 - DNA, 21 bp, Mus musculus
ggcactggatttggatgattt

SEQ ID NO: 62 - DNA, 64 bp, Artificial Sequence
  SYNTHESIZED
tgctgaaatcatccaaatccagtgccgttttggccactgactgacggcactggttggatgattt

SEQ ID NO: 63 - DNA, 21 bp, Mus musculus
cgaaggaattcttcccgctca

SEQ ID NO: 64 - DNA, 1113 bp, Rattus norvegicus
atgctgctccagagctgctctgcgtacaaactggtctgctactacaccaactggtcccagtaccgggaaggcaatgggagctgcttcccagatgccctcgaccattccctgtgcacccatatcatctacagctttgccaacatcagcaacaacaagctcagcacatcggagtggaatgacgtaaccctgtatggcatgctgaatactctcaagaccagaaaccccagactgaagacactgctgtctgttggaggatggagctttggctcagaaagattttccaggattgtctccaacgctaagagtcgcaagactttcgtccagtcggtagctcccttcctgcggacctatggctttgatggactggatctcgcctggctctacccgggcccgaaagacaagcaacattttaccacactgatcaaggaactgaaggcggaattcacaaaggaagtccagccaggcacagagaaactcctgctcagtgctgccgtgtcagcaggaaaggtgacccttgacagtggctatgatgttgcccagatagcccaacacctagatttcattaatctcatgacctatgatttccatggaacctggcgccacaccacaggacatcacagccccctcttccgaggccagcaggacactgggcctgacagattcagcaatgtggactatggtgtggggtacatgctaaggctgggagcccccaccaacaagctagtgatgggtatccccacctttggaaagagcttcactctggcatcttctgagaatcaagtgggagctccaatcacagggtcaggattaccaggccgctacaccaaggagaaagggaccctcgcctactacgagatatgcgacttcctcagaggagctgaagtacatagaattcttggccagcaggttccctttgctaccaagggcaaccagtgggtggggtatgatgacccggagagcgtcaaaaacaaggtgaagtacctgaagaacaagcagctggcaggagccatggtgtgggcagtggatttggatgatttccggggctccttctgtgggcataacgtacacttcccgctcaccaacgccatcaaggaggccctggctgtggcttag

SEQ ID NO: 65 - DNA, 21 bp, Rattus norvegicus
ctttcctgtgcacccacatca

SEQ ID NO: 66 - DNA, 64 bp, Artificial Sequence; identical to SEQ ID NO: 51
  SYNTHESIZED

SEQ ID NO: 67 - DNA, 20 bp, Rattus norvegicus
cgaactatgacaagctgaat

SEQ ID NO: 68 - DNA, 21 bp, Rattus norvegicus
cactgagagacgcactgcttt

SEQ ID NO: 69 - DNA, 21 bp, Rattus norvegicus
gcttaagagacaagcagtatt

SEQ ID NO: 70 - DNA, 21 bp, Rattus norvegicus
gaactgaatgcggaattcaca

SEQ ID NO: 71 - DNA, 64 bp, Artificial Sequence; identical to SEQ ID NO: 56
  SYNTHESIZED

SEQ ID NO: 72 - DNA, 21 bp, Rattus norvegicus
gcagctttgtcagcaggaaag

SEQ ID NO: 73 - DNA, 21 bp, Rattus norvegicus
ctaggtttgacagatacagca

SEQ ID NO: 74 - DNA, 21 bp, Rattus norvegicus
ttcctcaaaggagctgaagta

SEQ ID NO: 75 - DNA, 64 bp, Artificial Sequence; identical to SEQ ID NO: 60
  SYNTHESIZED

SEQ ID NO: 76 - DNA, 21 bp, Rattus norvegicus
ggcactggatttggatgattt

SEQ ID NO: 77 - DNA, 64 bp, Artificial Sequence; identical to SEQ ID NO: 62
  SYNTHESIZED

SEQ ID NO: 78 - DNA, 21 bp, Rattus norvegicus
cgaaggaattcttcccgctca

SEQ ID NO: 79 - DNA, 64 bp, Artificial Sequence
  SYNTHESIZED
tgctgagagtattcagcatgccatacgttttggccactgactgacgtatggcactgaatactct

SEQ ID NO: 80 - DNA, 64 bp, Artificial Sequence
  SYNTHESIZED
tgctgctcagaagatgccagagtgaagttttggccactgactgacttcactctcatcttctgag

SEQ ID NO: 81 - DNA, 64 bp, Artificial Sequence
  SYNTHESIZED
tgctgaaatcatccaaatccactgccgttttggccactgactgacggcagtggttggatgattt

SEQ ID NO: 82 - DNA, 64 bp, Artificial Sequence; identical to SEQ ID NO: 6
  SYNTHESIZED

SEQ ID NO: 83 - DNA, 28 bp, Artificial Sequence
  SYNTHESIZED
cagcggccgcatgctgctccagagctgc

SEQ ID NO: 84 - DNA, 30 bp, Artificial Sequence
  SYNTHESIZED
cagcggccgcatggtgcagtccctggcctt

SEQ ID NO: 85 - DNA, 29 bp, Artificial Sequence
  SYNTHESIZED
cagcggccgcatgggcatgagggcggcac

SEQ ID NO: 86 - DNA, 31 bp, Artificial Sequence
  SYNTHESIZED
cagcggccgcatggccaagctcattcttgtc

SEQ ID NO: 87 - DNA, 2247 bp, Artificial Sequence
  SYNTHESIZED
tgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccctagcgtttccggcgacggtgctagcgtcgaccagtggatcctggaggcttgctgaaggctgtatcaggacacaaggcctgttactagcactcacatggaacaaatggcccagatctggccgccaggacacaaggcctgttactagcactcacatggaacaaatggcccagatctggccgcactcgaaaacgggccctctagactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcg

SEQ ID NO: 88 - DNA, 717 bp, Artificial Sequence
  SYNTHESIZED
gttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctag

(19) United States
(12) Patent Application Publication
(10) Pub. No.: US 2024/0084323 A1
(43) Pub. Date: Mar. 14, 2024

(54) MODULATION OF CHITINASE PROTEIN EXPRESSION

(71) Applicant: Dignity Health, San Francisco, CA (US)

(72) Inventors: Fredric Manfredsson, San Francisco, CA (US); Robert Bowser, San Francisco, CA (US)

(21) Appl. No.: 18/261,319

(22) PCT Filed: Jan. 13, 2022
(86) PCT No.: PCT/US2022/012349

§ 371 Date: Jul. 13, 2023

Related U.S. Application Data
(60) Provisional application No. 63/136,983, filed on Jan. 13, 2021.

(51) Int. Cl. C12N 15/86; A61K 48/00; C12N 9/42; C12N 15/113

(52) U.S. Cl. CPC C12N 15/86; A61K 48/005; C12N 9/2442; C12N 15/1137; C12N 2310/141; C12N 2310/20; C12N 2750/14143

(57) ABSTRACT

Compositions and methods for modifying the expression of one or more chitinase proteins in a target cell or tissue type are disclosed. The compositions can be used in methods of treating a disease condition associated with expression of a chitinase protein in a cell or tissue type in a subject in need thereof, such as ALS and PD.

### CROSS REFERENCE TO RELATED APPLICATIONS

This application claims priority benefit from Provisional Application No. 63/136,983, filed Jan. 13, 2021, the contents of which are hereby incorporated by reference in their entirety.

### SEQUENCE LISTING

This application contains a Sequence Listing that has been submitted in ASCII format via EFS-Web and is hereby incorporated by reference in its entirety. The ASCII copy is named 102695_696917_sequencelisting_ST25.txt, and is 34.6 kilobytes in size.

### GOVERNMENTAL RIGHTS

This invention was made with government support under AL200061 awarded by the Department of Defense. The government has certain rights in the invention.

### FIELD OF THE INVENTION

The present disclosure provides compositions and methods for modifying the expression of one or more chitinase proteins in a target cell or tissue type.

### BACKGROUND OF THE INVENTION

Chitinases and Chitinase-Like Proteins (C/CLPs) have been shown to modulate innate immune responses, extracellular tissue remodeling, fibrosis and solid carcinomas, cell migration and differentiation, and function to modulate inflammation in the progression of many human diseases. For instance, inflammation, once thought to be the consequence of the neuronal death occurring in neurodegenerative disorders such as Alzheimer's disease (AD), Parkinson's disease (PD), and amyotrophic lateral sclerosis (ALS), is now increasingly thought of as playing a participatory role in the disease process itself. However, since chitinase proteins are expressed by multiple cell types throughout the body, a targeted approach (instead of a global knock-out model) is necessary to develop therapeutic tools for treating diseases associated with chitinase protein expression.

A need therefore exists for targeted modulation of endogenous chitinase expression as a modality for treating health conditions associated with chitinase protein expression.

### SUMMARY OF THE INVENTION

One aspect of the present disclosure encompasses a composition for modifying expression of one or more chitinase proteins in a target cell or tissue type. The chitinase protein can be SI-CLP (CHID1), YKL-39 (CHI3L2), YKL-40 (CHI3L1), Chitriosidase (Chit-1), AMCase (CHIA), or oviductin protein. The target cell or tissue type can be any cell or tissue type wherein expression of the chitinase protein is associated with a disease condition. For instance, the target cell or tissue type can be an organ in the body, a cell in the nervous system, a cancer cell, or tumor, or a cell of the immune system. In some aspects, the target cell or tissue type is a cell of glial lineage. In some aspects, the target cell is a subset of activated glia. The target cell can also be activated astrocytes. Modifying the expression of the chitinase protein in a target cell or tissue type can decrease inflammation.

The composition comprises a nucleic acid construct encoding a modification system targeted to the one or more chitinase proteins. The expression modification system can modify the expression of the chitinase protein in the nervous system. For instance, the modification system can modify the expression of YKL-39 (CHI3L2), YKL-40 (CHI3L1), Chitriosidase (Chit-1), or any combination thereof in the nervous system.

The protein expression modification system can be a peptide, polypeptide, antibody, or antibody fragment. The expression modification system can also be a programmable nucleic acid modification system. The programmable nucleic acid modification system can be an interfering nucleic acid molecule such as an antisense molecule, siRNA molecules, single-stranded siRNA molecules, miRNA molecules, piRNA molecules, lncRNA molecules, shRNA molecules, or any combination thereof.

In some aspects, the programmable nucleic acid modification system is a nucleic acid editing system. The nucleic acid editing system can be an RNA-guided clustered regularly interspersed short palindromic repeats (CRISPR)/CRISPR-associated (Cas) (CRISPR/Cas) nuclease system, a CRISPR/Cpf1 nuclease system, a zinc finger nuclease (ZFN), a transcription activator-like effector nuclease (TALEN), a meganuclease, ribozyme, a programmable DNA binding domain linked to a nuclease domain, or any combination thereof. In some aspects, the programmable nucleic acid modification system is a CRISPR/Cas tool modified for transcriptional regulation of a locus. The programmable nucleic acid modification system can be a CRISPR/Cas transcriptional regulator driven by cell specific promoters using a catalytically dead effector (dCAS9) to modulate transcription of a chitinase gene encoding the chitinase protein. The system for targeting the nucleic acid construct to a target cell or tissue can be a viral vector.

The composition also comprises a nucleic acid delivery system for delivering the nucleic acid construct to the target cell or tissue. The delivery system can be an adeno-associated virus (AAV) vector encapsidating the nucleic acid construct for delivering the construct to the target cell or tissue type.

In some aspects, the protein expression modification system modifies the expression of one or more chitinase proteins to decrease the inflammatory profile of the one or more chitinase genes in distinct glial subsets. The target cell or tissue type can be in a subject having ALS.

When the subject has ALS, the protein expression modification system can modify the expression of Chit-1, CHI3L1, CHI3L2, or any combination thereof in the central nervous system. When the subject has ALS, the protein expression modification system can also modify the expression of Chit-1, CHI3L1, CHI3L2, or any combination thereof in a cell of glial lineage. In some aspects, when the subject has ALS, the protein expression modification system reduces the expression of Chit-1 in activated microglia, increases the expression of CHI3L1 in activated astrocytes, increases the expression of CHI3L2 in microglia, or any combination thereof in subpopulations of glial cells, and the nucleic acid delivery system is an AAV vector having tropism to the subsets of activated glial cells.

In some aspects, the target cell or tissue type is in a subject having PD. When the subject has PD, the protein expression modification system can reduce the expression of CHI3L1 in activated astrocytes.

Another aspect of the present disclosure encompasses a method of treating a disease condition associated with expression of a chitinase protein in a cell or tissue type in a subject in need thereof. The method comprises modifying the expression of one or more chitinase proteins in the cell or tissue type in the subject by administering to the subject a therapeutically effective amount of a composition for modifying expression of one or more chitinase proteins in a target cell or tissue type. The composition can be as described herein above. The disease condition can be a condition associated with inflammation. The disease condition can also be a neurological condition. In some aspects, the disease condition is a cancer.

Yet another aspect of the present disclosure encompasses a method of treating a neurological condition associated with expression of a chitinase protein in a cell or tissue type in the nervous system in a subject in need thereof. The method comprises modifying the expression of one or more chitinase proteins in the cell or tissue type in the nervous system of the subject by administering to the subject a therapeutically effective amount of a composition for modifying expression of one or more chitinase proteins in a target cell or tissue type. The composition can be as described herein above. The protein expression modification system can modify the expression of one or more chitinase proteins in the nervous system. In some aspects, the neurological condition is Alzheimer's disease (AD), Parkinson's disease (PD), Amyotrophic lateral sclerosis (ALS), multiple sclerosis (MS), ataxia, Bell's palsy, or epilepsy. In some aspects, the modification system modifies the expression of one or more chitinase proteins to decrease the inflammatory profile of the one or more chitinase proteins in distinct glial subsets. The expression modification system can modify the expression of Chit-1, CHI3L1, CHI3L2, or any combination thereof in the central nervous system.

In some aspects, the neurological condition is ALS. When the neurological condition is ALS, the expression modification system modifies the expression of one or more chitinase proteins in activated glial subtypes. The modification system can modify the expression of Chit-1, CHI3L1, CHI3L2, or any combination thereof in a cell of glial lineage. Further, when the neurological condition is ALS, the protein expression modification system reduces the expression of Chit-1 in activated microglia, reduces the expression of CHI3L1 in activated astrocytes, reduces the expression of CHI3L2 in activated microglia, or any combination thereof.

In some aspects, the neurological condition is Parkinson's disease (PD). When the neurological condition is PD, the protein expression modification system reduces the expression of CHI3L1 protein in activated astrocytes.

One aspect of the present disclosure encompasses a method of treating amyotrophic lateral sclerosis (ALS) in a subject in need thereof. The method comprises modifying the expression of one or more chitinase proteins in a cell or tissue type associated with ALS in the nervous system of the subject by administering to the subject a therapeutically effective amount of a composition for modifying expression of one or more chitinase proteins in a target cell or tissue type. The composition can be as described herein above.

The modification system can modify the expression of one or more chitinase proteins in activated glial subtypes. For instance, the modification system can modify the expression of Chit-1, CHI3L1, CHI3L2, or any combination thereof in the central nervous system. The protein expression modification system can also modify the expression of Chit-1, CHI3L1, CHI3L2, or any combination thereof in a cell of glial lineage. The protein expression modification system can reduce the expression of Chit-1 in activated microglia, reduce the expression of CHI3L1 in activated astrocytes, reduce the expression of CHI3L2 in activated microglia, or any combination thereof.

An additional aspect of the present disclosure encompasses a method of treating Parkinson's disease (PD) in a subject in need thereof. The method comprises modifying the expression of one or more chitinase proteins in a cell or tissue type associated with PD in the nervous system of the subject by administering to the subject a therapeutically effective amount of a composition for modifying expression of one or more chitinase proteins in a target cell or tissue type. The composition can be as described herein above. In some aspects, the protein expression modification system modifies the expression of one or more chitinase proteins in activated astrocytes. In some aspects, the modification system reduces the expression of CHI3L1 proteins in activated astrocytes.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Astrocyte CHI3L1 expression is increased in the midbrain of PD patients. Panel A) Midbrain sections from a PD patient (Age 80, H&Y 4), or panel B) Age-matched control were stained for CHI3L1. Strong CHI3L1 expression was observed in cells with astrocyte-like morphology (black) in the PD SN, whereas little CHI3L1 was seen in an age-matched control nigra. Inset in A shows an example of a strongly positive CHI3L1 cell “surrounding” a neuromelanin-containing cell.

FIG. 2. CHI3L1 expression is increased prior to dopamine neuron loss. Rats were treated with either AAV-GFP (panels A and C) or AAV human α-syn (panels B, D and E). A, B, CHI3L1 expression in the treated SN. Panels C and D Fluorescent imaging shows colocalization of CHI3L1 (blue) with GFAP (red). Panel E area outlined in (panel D) shows CHI3L1 positive astrocyte flanking a human α-syn overexpressing neuron.

FIG. 3. Triple label confocal microscopy for GFAP (green), CHI3L1 (red), and DAPI (blue) in the motor cortex white matter of an ALS patient. White arrowhead denotes a GFAP positive astrocyte and yellow arrowhead denotes a GFAP- and CHI3LI-positive astrocyte. Bar=20 μm.

FIG. 4. Double label confocal microscopy for GFAP (green), CHI3L1 (red), and DAPI (blue) in the subpial surface of ALS motor cortex. Extensive overlap between GFAP and CHI3L1 staining in this spatial location.

FIG. 5. Triple label confocal microscopy for GFAP (green), IBA-1 (pseudocolor white), Chit-1 (red), and DAPI (blue) in the motor cortex gray matter of an ALS patient. Chit-1 immunoreactivity co-localizes with IBA-1 positive activated microglia but not GFAP positive astrocytes.

FIG. 6. BRP-39 immunostaining in the cortex (left) and cerebellum (right) of transgenic SOD1 G93A mice at time of disease onset (top panels) and age and gender matched non-transgenic control mice (bottom panels). BRP-39 is labeled red with Hematoxylin counterstain. Note increased BRP-39 IHC in glia in the cortex and cerebellar white matter. Images using 10× objective.

FIG. 7 is an illustration of Chitins (C) and chitin-like protein (CLPs) regulatory functions.

FIG. 8 is an illustration of chitinases and chitinase-like proteins being researched.

FIG. 9 is an illustration of steps to identify and characterize chitinase expression in post-mortem brain sections.

FIG. 10A are the immunohistochemistry results of chitinase expression in post-mortem brain sections of Healthy Control Subjects. Scale Bar represents 100 μm in low magnification images and 10 μm in high magnification inset.

FIG. 10B are the immunohistochemistry results of chitinase expression in post-mortem brain sections of PD Subjects. Scale Bar represents 100 μm in low magnification images and 10 μm in high magnification inset.

FIG. 10C are the immunohistochemistry results of chitinase expression in post-mortem brain sections of rats injected with AAV2/5 expressing GFP. Scale Bar represents 100 μm in low magnification images and 10 μm in high magnification inset.

FIG. 10D are the immunohistochemistry results of chitinase expression in post-mortem brain sections of rats injected with AAV2/5 expressing alpha-synuclein. Scale Bar represents 100 μm in low magnification images and 10 μm in high magnification inset.

FIG. 11 are the histological analysis results showing that the pattern of CHI3L1 in situ hybridization (ISH) is more abundant than CHIT-1 in the SN of both GFP (panels A-D) and a-Syn (panels E-H) injected rats. The histological analysis results showing that CHI3L1 ISH is more abundant in a-Syn (panels E-H) injected rats compared to GFP controls (panels A-D). Scale Bar represents 100 μm in low magnification images (panels A, E) and 10 μm in high magnification images (panels B, C, D, F, G, H).

FIG. 12 are the immunohistochemistry results of in situ hybridization expression of CHI3L1 and CHIT-1 in the substantia nigra. Scale Bar represents 100 μm in low magnification images (A, E) and 10 μm in high magnification images (B, C, D, F, G, H).

FIG. 13 depicts the steps to modulate chitinase expression in rats

FIG. 14 are luciferase activities caused by engineered genomes for knocking down CHI3L1 expression in mice.

FIG. 15 are luciferase activities caused by engineered genomes for knocking down CHI3L1 expression in rats.

FIG. 16 is the genome mapping of constructed miR907.

### DETAILED DESCRIPTION

The present disclosure relates to compositions and methods for modifying the expression of one or more chitinase proteins in a target cell or tissue type. The inventors surprisingly discovered that modifying the expression of one or more chitinase proteins in a target cell or tissue type can treat health conditions associated with chitinase expression. More specifically, the inventors discovered that reducing the expression of chitinases in a target tissue can treat health conditions associated with chitinase expression. The compositions and methods of the instant disclosure can modify chitinase expression in specific target cells or tissue types, thereby allowing for treatment of a health condition all while reducing or eliminating any possible effects of an approach directed to global modification of the chitinases.

### I. Composition

One aspect of the present disclosure encompasses a composition for modifying the expression of one or more chitinase proteins in a target cell or tissue type.

### (a) Chitinases

Chitinases and chitinase-like proteins, collectively referred to as chitinase proteins, are members of the glycoside hydrolase family 18 and function to degrade chitin, modulate innate immune responses, cell migration and differentiation, and modulate inflammation in the progression of many human diseases. To date, six chitinase or chitinase-like proteins have been identified in humans: CHID1 (SI-CLP), CHI3L2 (YKL-39), CHI3L1 (YKL-40), Chitriosidase (Chit-1), AMCase (CHIA), and oviductin. Chitotriosidase (Chit-1) was the first identified mammalian chitinase protein that both binds and degrades chitin. Some members of the chitinase family, such as chitinase-3− like protein 1 (CHI3L1) or chitinase-3− like protein 2 (CHI3L2), bind chitin but do not exhibit enzymatic activity. Additional chitinase proteins such as YM1 and YM2 (Chi3l3/l4) have been identified in certain non-human mammals. Accordingly, a composition of the instant disclosure can modify the expression of a SI-CLP protein, YKL-39 (CHI3L2) protein, YKL-40 (CHI3L1) protein, Chitriosidase (Chit-1) protein, AMCase (CHIA) protein, oviductin protein, YM1 protein, YM2 protein, or any combination thereof.

In some aspects, the chitinase protein is YM2. In some aspects, the chitinase protein is a human chitinase protein. In some aspects, the chitinase is a human CHID1 protein. In some aspects, the chitinase is a human CHI3L2 protein. In some aspects, the chitinase is a human Chitriosidase protein. In some aspects, the chitinase is a human AMCase protein. In some aspects, the chitinase is a human oviductin protein.

In some aspects, the chitinase protein is a YM1 protein. In some aspects, the YM1 protein comprises an amino acid sequence encoded by a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 2. In some aspects, the YM1 protein comprises an amino acid sequence encoded by a nucleotide sequence comprising about 75% or more, 85% or more, 95% or more, or 100% sequence identity with the nucleic acid sequence of SEQ ID NO: 2.

In some aspects, the chitinase protein is a Chit-1 protein. In some aspects, the Chit-1 protein comprises an amino acid sequence encoded by a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 17. In some aspects, the Chit-1 protein comprises an amino acid sequence encoded by a nucleotide sequence comprising about 75% or more, 85% or more, 95% or more, or 100% sequence identity with the nucleic acid sequence of SEQ ID NO: 17.

In some aspects, the chitinase protein is a Chit-1 protein. In some aspects, the Chit-1 protein comprises an amino acid sequence encoded by a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 33. In some aspects, the Chit-1 protein comprises an amino acid sequence encoded by a nucleotide sequence comprising about 75% or more, 85% or more, 95% or more, or 100% sequence identity with the nucleic acid sequence of SEQ ID NO: 33.

In some aspects, the chitinase protein is a CHI3L1 protein. In some aspects, the CHI3L1 protein comprises an amino acid sequence encoded by a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 33. In some aspects, the CHI3L1 protein comprises an amino acid sequence encoded by a nucleotide sequence comprising about 75% or more, 85% or more, 95% or more, or 100% sequence identity with the nucleic acid sequence of SEQ ID NO: 33.

In some aspects, the chitinase protein is a CHI3L1 protein. In some aspects, the CHI3L1 protein comprises an amino acid sequence encoded by a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 64. In some aspects, the CHI3L1 protein comprises an amino acid sequence encoded by a nucleotide sequence comprising about 75% or more, 85% or more, 95% or more, or 100% sequence identity with the nucleic acid sequence of SEQ ID NO: 64.

### (b) Target Cell or Tissue Type

The composition modifies the expression of the one or more chitinase proteins in a target cell or tissue type. The target cell or tissue type can be any cell or tissue type wherein expression of the one or more chitinase proteins is associated with a disease condition. Disease conditions are described further herein below in Section II(b).

The target cell or tissue type can be an organ in the body, the nervous system, including the central nervous system and the peripheral nervous system, a cancer cell or tumor, or cell of the immune system. In some aspects, the target cell or tissue type is the central nervous system. Non-limiting examples of cells in the nervous system include axons, oligodendrocytes, neuroblasts, neurons, glial cells, and astrocytes. In some aspects, the target cell or tissue type is a glial cell. In other aspects, the target cell or tissue type is an astrocyte. In some aspects, the target cell or tissue type is a cell of glial lineage. In some aspects, the target cell or tissue type is an activated glia. In other aspects, the target cell or tissue type is activated astrocytes.

In some aspects, the modification system modifies the expression of Chit-1, CHI3L1, CHI3L2, or any combination thereof in the nervous system. In some aspects, the expression modification system modifies the expression of Chit-1, CHI3L1, CHI3L2, or any combination thereof in a glial cell. In some aspects, the expression modification system modifies the expression of Chit-1, CHI3L1, CHI3L2, or any combination thereof in a cell of glial lineage. In some aspects, the expression modification system modifies the expression of Chit-1, CHI3L1, CHI3L2, or any combination thereof in an activated glia. In some aspects, the expression modification system modifies the expression of Chit-1, CHI3L1, CHI3L2, or any combination thereof in astrocytes.

In some aspects, the target cell or tissue type is in a subject having ALS. When the subject is a subject having ALS, the modification system modifies the expression of one or more chitinase proteins to decrease the inflammatory profile of the one or more chitinase proteins in the central nervous system. For instance, the protein expression modification system can modify the expression of Chit-1, CHI3L1, CHI3L2, or any combination thereof to decrease the inflammatory profile of the one or more chitinase proteins in the central nervous system. In some aspects, the modification system modifies the expression of one or more chitinase proteins to decrease the inflammatory profile of the one or more chitinase proteins in distinct glial subsets. In some aspects, when the subject has ALS, the protein expression modification system modifies the expression of Chit-1, CHI3L1, CHI3L2, or any combination thereof in a cell of glial lineage. In other aspects, when the subject has ALS, the protein expression modification system reduces the expression of Chit-1 in activated microglia, increases the expression of CHI3L1 in activated astrocytes, increases the expression of CHI3L2 in microglia, or any combination thereof in subpopulations of glial cell.

In some aspects, the target cell or tissue type is in a subject having PD. When the subject is a subject having PD, the modification system modifies the expression of one or more chitinase proteins to decrease the inflammatory profile of the one or more chitinase proteins in the central nervous system. For instance, the protein expression modification system can modify the expression of Chit-1, CHI3L1, CHI3L2, or any combination thereof to decrease the inflammatory profile of the one or more chitinase proteins in the central nervous system. In some aspects, the modification system modifies the expression of one or more chitinase proteins to decrease the inflammatory profile of the one or more chitinase proteins in activated astrocytes. In some aspects, when the subject has PD, the protein expression modification system modifies the expression of Chit-1, CHI3L1, CHI3L2, or any combination thereof in activated astrocytes. In some aspects, the target cell or tissue type is in a subject having PD. In some aspects, when the subject has PD, the protein expression modification system reduces the expression of a CHI3L1 protein in astrocytes. In some aspects, the astrocytes are activated astrocytes.

In some aspects, the target cell or tissue type is cancer cells. In some aspects, the cancer cell is glioblastoma, colon cancer, ovarian cancer, breast cancer, prostate cancer, osteosarcoma, or malignant melanoma. Other non-limiting examples of neoplasms or cancer cells that may be diagnosed using methods of the instant disclosure include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood).

In some aspects, the target cell or tissue type is immune cells such as lymphocutes, neutrophis, microglia, and monocytes/macrophages, or any combination thereof. In some aspects, the target cell or tissue type is monocytes, microglia.

### (c) Protein Expression Modification System

Any protein expression modification system capable of modifying the expression of the chitinase protein of interest can be used in the instant disclosure. Non-limiting examples of suitable protein expression modification systems include a programmable nucleic acid modification system, or a peptide, polypeptide, antibody, or antibody fragment which when expressed in the target cell or tissue type modifies the activity of the protein.

As used herein, “protein expression” includes but is not limited to one or more of the following: transcription of a gene into precursor mRNA; splicing and other processing of the precursor mRNA to produce mature mRNA; mRNA stability; translation of the mature mRNA into protein (including codon usage and tRNA availability); production of a mutant protein comprising a mutation that modifies the activity of the protein, including the calcium channel activity; and glycosylation and/or other modifications of the translation product, if required for proper expression and function. Non-limiting examples of suitable protein expression modification systems include programmable nucleic acid modification systems, or peptide, polypeptide, antibody, or antibody fragments which when expressed in a target cell or tissue type reduce the level of chitinase protein or the calcium channel activity of the protein.

In some aspects, the protein expression modification system is a programmable nucleic acid modification system targeted to a sequence within a gene encoding the chitinase protein. As used herein, a “programmable nucleic acid modification system” is a system capable of targeting and modifying the nucleic acid, or modifying the expression or stability of a nucleic acid to alter a protein or the expression of a protein encoded by the nucleic acid. The programmable nucleic acid modification system can comprise an interfering nucleic acid molecule or a nucleic acid editing system. The programmable protein expression modification system is specifically targeted to a sequence within a gene encoding the chitinase protein.

In some aspects, the programmable expression modification system comprises an interfering nucleic acid (RNAi) molecule having a nucleotide sequence complementary to a target sequence within a gene encoding the chitinase protein used to inhibit expression of the chitinase protein. RNAi molecules generally act by forming a heteroduplex with a target RNA molecule, which is selectively degraded or “knocked down,” hence inactivating the target RNA. Under some conditions, an interfering RNA molecule can also inactivate a target transcript by repressing transcript translation and/or inhibiting transcription. An interfering RNA is more generally said to be “targeted against” a biologically relevant target, such as a protein, when it is targeted against the nucleic acid encoding the target. For example, an interfering RNA molecule has a nucleotide (nt) sequence which is complementary to an endogenous mRNA of a target gene sequence. Thus, given a target gene sequence, an interfering RNA molecule can be prepared which has a nucleotide sequence at least a portion of which is complementary to a target gene sequence. When introduced into cells, the interfering RNA binds to the target mRNA, thereby functionally inactivating the target mRNA and/or leading to degradation of the target mRNA.

Interfering RNA molecules include, inter alia, small interfering RNA (siRNA), microRNA (miRNA), piwi-interacting RNA (piRNA), long non-coding RNAs (long ncRNAs or lncRNAs), and small hairpin RNAs (shRNA). lncRNAs are widely expressed and have key roles in gene regulation. Depending on their localization and their specific interactions with DNA, RNA and proteins, lncRNAs can modulate chromatin function, regulate the assembly and function of membraneless nuclear bodies, alter the stability and translation of cytoplasmic mRNAs, and interfere with signalling pathways. Piwi-interacting RNA (piRNA) is the largest class of small non-coding RNA molecules expressed in animal cells. piRNAs regulate gene expression through interactions with piwi-subfamily Argonaute proteins. SiRNA are double-stranded RNA molecules, preferably about 19-25 nucleotides in length. When transfected into cells, siRNA inhibit the target mRNA transiently until they are also degraded within the cell. MiRNA and siRNA are biochemically and functionally indistinguishable. Both are about the same in nucleotide length with 5′-phosphate and 3′-hydroxyl ends, and assemble into an RNA-induced silencing complex (RISC) to silence specific gene expression. siRNA and miRNA are distinguished based on origin. siRNA is obtained from long double-stranded RNA (dsRNA), while miRNA is derived from the double-stranded region of a 60-70nt RNA hairpin precursor. Small hairpin RNAs (shRNA) are sequences of RNA, typically about 50-80 base pairs, or about 50, 55, 60, 65, 70, 75, or about 80 base pairs in length, that include a region of internal hybridization forming a stem loop structure consisting of a base-pair region of about 19-29 base pairs of double-strand RNA (the stem) bridged by a region of single-strand RNA (the loop) and a short 3′ overhang. shRNA molecules are processed within the cell to form siRNA which in turn knock down target gene expression. shRNA can be incorporated into plasmid vectors and integrated into genomic DNA for longer-term or stable expression, and thus longer knockdown of the target mRNA.

Interfering nucleic acid molecules can contain RNA bases, non-RNA bases, or a mixture of RNA bases and non-RNA bases. For example, interfering nucleic acid molecules provided herein can be primarily composed of RNA bases but also contain DNA bases or non-naturally occurring nucleotides. The interfering nucleic acids can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone.

In some aspects, the chitinase protein is YM1, and the programmable nucleic acid modification system is an miRNA molecule comprising a nucleotide sequence complementary to a target sequence within the nucleotide sequence encoding the YM1 protein. In some aspects, the YM1 protein comprises an amino acid sequence encoded by a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 2. In some aspects, the YM1 protein comprises an amino acid sequence encoded by a nucleotide sequence comprising about 75% or more, 85% or more, 95% or more, or 100% sequence identity with the nucleic acid sequence of SEQ ID NO: 2. In some aspects, the miRNA molecule comprises a nucleotide sequence selected from SEQ ID NOs: 4, 6, 11, 14, 87, and any combination thereof, and the target sequence within the gene encoding the YM1 protein is selected from SEQ ID NOs: 3, 5, 7-10, 12, 13, and any combination thereof.

In some aspects, the chitinase protein is Chit-1, and the programmable nucleic acid modification system is an miRNA molecule comprising a nucleotide sequence complementary to a target sequence within the nucleotide sequence encoding the Chit-1 protein. In some aspects, the Chit-1 protein comprises an amino acid sequence encoded by a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 17. In some aspects, the Chit-1 protein comprises an amino acid sequence encoded by a nucleotide sequence comprising about 75% or more, 85% or more, 95% or more, or 100% sequence identity with the nucleic acid sequence of SEQ ID NO: 17. In some aspects, the miRNA molecule comprises a nucleotide sequence selected from SEQ ID NOs: 20, 23, 25, 29, 31, and any combination thereof, and the target sequence within the gene encoding the Chit-1 protein is selected from SEQ ID NOs: 18, 19, 21, 22, 24, 26-28, 30, 32, and any combination thereof.

In some aspects, the chitinase protein is Chit-1, and the programmable nucleic acid modification system is an miRNA molecule comprising a nucleotide sequence complementary to a target sequence within the nucleotide sequence encoding the Chit-1 protein. In some aspects, the Chit-1 protein comprises an amino acid sequence encoded by a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 33. In some aspects, the Chit-1 protein comprises an amino acid sequence encoded by a nucleotide sequence comprising about 75% or more, 85% or more, 95% or more, or 100% sequence identity with the nucleic acid sequence of SEQ ID NO: 33. In some aspects, the miRNA molecule comprises a nucleotide sequence selected from SEQ ID NOs: 35, 38, 40, 43, 46, and any combination thereof, and the target sequence within the gene encoding the Chit-1 protein is selected from SEQ ID NOs: 34, 36, 37, 39, 41, 42, 44, 45, 47, 48, and any combination thereof.

In some aspects, the chitinase protein is CHI3L1, and the programmable nucleic acid modification system is an miRNA molecule comprising a nucleotide sequence complementary to a target sequence within the nucleotide sequence encoding the CHI3L1 protein. In some aspects, the CHI3L1 protein comprises an amino acid sequence encoded by a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 49. In some aspects, the CHI3L1 protein comprises an amino acid sequence encoded by a nucleotide sequence comprising about 75% or more, 85% or more, 95% or more, or 100% sequence identity with the nucleic acid sequence of SEQ ID NO: 49. In some aspects, the miRNA molecule comprises a nucleotide sequence selected from SEQ ID NOs: 51, 56, 60, 62, and any combination thereof, and the target sequence within the gene encoding the CHI3L1 protein is selected from SEQ ID NOs: 50, 52-55, 57-59, 61, 63, and any combination thereof.

In some aspects, the chitinase protein is CHI3L1, and the programmable nucleic acid modification system is an miRNA molecule comprising a nucleotide sequence complementary to a target sequence within the nucleotide sequence encoding the CHI3L1 protein. In some aspects, the CHI3L1 protein comprises an amino acid sequence encoded by a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 64. In some aspects, the CHI3L1 protein comprises an amino acid sequence encoded by a nucleotide sequence comprising about 75% or more, 85% or more, 95% or more, or 100% sequence identity with the nucleic acid sequence of SEQ ID NO: 64. In some aspects, the miRNA molecule comprises a nucleotide sequence selected from SEQ ID NOs: 66, 71, 75, 77, and any combination thereof, and the target sequence within the gene encoding the CHI3L1 protein is selected from SEQ ID NOs: 65, 67-70, 72-74, 76, 78, and any combination thereof.

In some aspects, the programmable nucleic acid modification system is a nucleic acid editing system. Such modification system can be used to edit DNA or RNA sequences to repress transcription or translation of an mRNA encoded by the gene, and/or produce mutant proteins with reduced activity or stability. Non-limiting examples of programmable nucleic acid editing systems include, without limit, an RNA-guided clustered regularly interspersed short palindromic repeats (CRISPR)/CRISPR-associated (Cas) (CRISPR/Cas) nuclease system, a CRISPR/Cpf1 nuclease system, a zinc finger nuclease (ZFN), a transcription activator-like effector nuclease (TALEN), a meganuclease, a ribozyme, or a programmable DNA binding domain linked to a nuclease domain. Other suitable programmable nucleic acid modification systems will be recognized by individuals skilled in the art.

In some aspects, the programmable nucleic acid modification system is a CRISPR/Cas tool modified for transcriptional regulation of a locus. In some aspects, the programmable nucleic acid modification system is a CRISPR/Cas transcriptional regulator driven by cell-specific promoters using a catalytically dead effector (dCAS9) to modulate transcription of a chitinase gene encoding a chitinase protein.

In some aspects, the programmable nucleic acid modification system is a programmable nucleic acid editing system. Such modification systems can be engineered to edit specific DNA or RNA sequences to repress transcription or translation of an mRNA encoded by the gene, and/or produce mutant proteins with reduced activity or stability. Non-limiting examples of programmable nucleic acid editing systems include, without limit, an RNA-guided clustered regularly interspersed short palindromic repeats (CRISPR) system, such as a CRISPR− associated (Cas) (CRISPR/Cas) nuclease system, a CRISPR/Cpf1 nuclease system, a zinc finger nuclease (ZFN) system, a transcription activator-like effector nuclease (TALEN) system, or a system comprising a meganuclease, a ribozyme, or a programmable DNA binding domain linked to a nuclease domain. Other suitable programmable nucleic acid modification systems will be recognized by individuals skilled in the art. Such systems rely for specificity on the delivery of exogenous protein(s), and/or a guide RNA (gRNA) or single guide RNA (sgRNA) having a sequence which binds specifically to a gene sequence of interest. The system components are delivered by a plasmid or viral vector or as a synthetic oligonucleotide. For example, engineered CRISPR systems comprise a gRNA or sgRNA, and a CRISPR-associated endonuclease. The gRNA is a short synthetic RNA comprising a sequence necessary for endonuclease binding, and a preselected ˜20 nucleotide spacer sequence targeting the sequence of interest in a genomic target. Systems such as ZFNs and TALENs rely on customizable sequence-specific DNA-binding domains which are connected to a nonspecific nuclease for target cleavage.

Nucleases that can be used in programmable nucleic acid editing systems include any endonuclease that cleaves phosphodiester bonds within a polynucleotide. An endonuclease may specifically cleave phosphodiester bonds within a DNA polynucleotide. In some embodiments, an endonuclease is a ZFN, a TALEN, a homing endonuclease (HE), a meganuclease, a MegaTAL, or a CRISPR-associated endonuclease. In some aspects, an endonuclease is a RNA-guided endonuclease. In certain aspects, an RNA-guided endonuclease can be a CRISPR nuclease, e.g., a Type II CRISPR Cas9 endonuclease or a Type V CRISPR Cpf1 endonuclease. In some embodiments, an endonuclease is a Cas, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or any combination thereof. In some embodiments, an endonuclease may introduce one or more single-stranded breaks (SSBs) and/or one or more double-stranded breaks (DSBs).

In some aspects, the programmable nucleic acid modification system is a CRISPR/Cas tool modified for transcriptional regulation of a locus. In some aspects, the programmable nucleic acid modification system is a CRISPR/Cas transcriptional regulator driven by cell-specific promoters using a catalytically dead effector (dCAS9) to modulate transcription of a chitinase gene encoding a chitinase protein.

### (d) Delivery Vectors

The composition of the instant disclosure comprises a nucleic acid delivery vector for delivering the nucleic acid construct to the target cell or tissue. The nucleic acid delivery vector can be any vector capable of delivering the nucleic acid construct to the target cell or tissue. Non-limiting examples of delivery vectors include viral and non-viral constructs, and/or vectors to introduce the programmable nucleic acid modification system into a cell or organism. In some aspects, the delivery has tropism to the target or tissue type.

In some aspects, the nucleic acid delivery vector is a non-viral vector. Vectors can include plasmids, linear DNA fragments, viruses, bacteriophage, pro-viruses, phagemids, transposons, and artificial chromosomes and the like, that may or may not be able to replicate autonomously or integrate into a chromosome of a host cell. Such vectors can be delivered to a cell or tissue by electroporation, using a variety of means. Suitable delivery means include synthetic oligonucleotides, lipoplexes, polymersomes, polyplexes, dendrimers, inorganic nanoparticles, cell-penetrating peptides, microinjection, electroporation, sonoporation, biolistics, calcium phosphate-mediated transfection, cationic transfection, liposomes and other lipids, dendrimer transfection, heat shock transfection, nucleofection transfection, gene gun delivery, dip transformation, supercharged proteins, cell-penetrating peptides, implantable devices, magnetofection, lipofection, impalefection, optical transfection, proprietary agent-enhanced uptake of nucleic acids, and delivery via liposomes, immunoliposomes, virosomes, or artificial virions.

In some aspects, the nucleic acid delivery vector is a viral vector. The viral vector may be an adenovirus vector; an adeno-associated virus (AAV) vector; a pox virus vector, such as a fowlpox virus vector; an alpha virus vector; a baculoviral vector; a herpes virus vector; a retrovirus vector, such as a lentivirus vector; a Modified Vaccinia virus Ankara vector; a Ross River virus vector; a Sindbis virus vector; a Semliki Forest virus vector; and a Venezuelan Equine Encephalitis virus vector.

In some aspects, the vector is a lentiviral vector. A recombinant lentiviral vector is capable of transducing a target cell with a nucleotide of interest. Once within the cell, the RNA genome from the vector particle is reverse transcribed into DNA and integrated into the genome of the target cell. The lentiviral vector can be derived from or may be derivable from any suitable lentivirus. The lentiviral vector can be derived from primate or non-primate lentiviruses. Examples of primate lentiviruses include but are not limited to the human immunodeficiency virus (HIV) and the simian immunodeficiency virus (SrV). The non-primate lentiviral group includes the prototype “slow virus” visna/maedi virus (VMV), as well as the related caprine arthritis-encephalitis virus (CAEV), equine infectious anemia virus (EIAV), feline immunodeficiency virus (FIV), and bovine immunodeficiency virus (BIV).

In other aspects, the viral vector is a herpes simplex virus (HSV) vector. The genome of the type-1 (HSV-1) is about 150 kb of linear, double-stranded DNA, featuring about 70 genes. Many viral genes may be deleted without the virus losing its ability to propagate. The “immediately early” (IE) genes are transcribed first. They encode trans-acting factors which regulate expression of other viral genes. The “early” (E) gene products participate in replication of viral DNA. The late genes encode the structural components of the virion as well as proteins, which turns on transcription of the IE and E genes, or disrupt host cell protein translation.

In yet other aspects, the delivery vector is a recombinant adeno-associated virus (rAAV) vector comprising the nucleic acid expression construct for delivering the construct to the target cell or tissue type. The adenovirus genome consists of about 36 kb of double-stranded DNA. Adenoviruses target airway epithelial cells, but are also capable of infecting neurons. Recombinant adenovirus vectors have been used as gene transfer vehicles for non-dividing cells. These vectors are similar to recombinant HSV vectors, since the adenovirus E1a immediate-early gene is removed but most viral genes are retained. Since the E1a gene is small (roughly 1.5 kb) and the adenovirus genome is approximately one-third the size of the HSV genome, other non-essential adenovirus genes are removed in order to insert a foreign gene within the adenovirus genome.

The AAV genome is built of single-stranded deoxyribonucleic acid (ssDNA), either positive- or negative-sensed, which is about 4.7 kilobase long. The genome comprises inverted terminal repeats (ITRs) at both ends of the DNA strand, and two open reading frames (ORFs): rep and cap. The former is composed of four overlapping genes encoding Rep proteins required for the AAV life cycle, and the latter contains overlapping nucleotide sequences of capsid proteins: VP1, VP2 and VP3, which interact to form a capsid with icosahedral symmetry. With regard to use of rAAVs as vectors for nucleic acid delivery into cells, ITRs can be sequences sufficient in cis next to desired gene to be delivered into a cell. Structural (cap) and packaging (rep) proteins can be delivered in trans. Many methods are established for efficient production of recombinant AAV (rAAV) vectors containing a reporter or a gene of interest. Accordingly, rAVs are constructed by inserting a desired gene together with a promoter to drive transcription of the gene in the AAV genome between the inverted terminal repeats (ITRs) that aid in concatemer formation in the nucleus after the single-stranded vector DNA is converted by host cell DNA polymerase complexes into double-stranded DNA.

In some aspects, the nucleic acid delivery vector is an rAAV vector comprising a nucleic acid expression construct inserted between the ITRs of an AAV virus genome. In some aspects, the rAAV vector comprises a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 88. In some aspects, the rAAV vector comprises a nucleotide sequence comprising about 75% or more, 85% or more, 95% or more, or 100% sequence identity with the nucleotide sequence of SEQ ID NO: 88.

Viral vectors are generally encapsidated in the viral capsid protein(s) for introduction into cells. Viral vectors can be targeted to specific cell types or tissue by encapsidating the viral vectors in virions using capsid proteins having tropism to (or that can infect) the specific cell type or tissue. For instance, capsid protein of AAV serotype 2 presents natural tropism towards skeletal muscles, neurons, vascular smooth muscle cells and hepatocytes. Capsid protein of AAV serotype 6 appears much better in infecting airway epithelial cells, capsid protein of AAV serotype 7 presents very high transduction rate of murine skeletal muscle cells (similar to capsid protein of AAV serotype 1 and capsid protein of AAV serotype 5), and capsid protein of AAV serotype 8 transduces hepatocytes.

In some aspects, a delivery vector has tropism to a desired target cell or tissue type. In some aspects, the target cell or tissue type is the central nervous system. The use of rAAV vectors to deliver nucleic acids into the brain is well known in the art. (See, e.g., U.S. Pat. No. 8,487,088, which is incorporated by reference herein in its entirety). The AAV can be any AAV serotype, including a serotype selected from AAV1, AAV2, AAV3, AAV4, AAV5, AAV6, AAV7, AAV8, AAVrh8, AAVrh10, AAVrh39, or AAVrh43. Included are AAVs with incorporated mutations inhibiting the canonical HSPG binding site (e.g. R585, R588, R487), or improving intracellular trafficking (e.g. Y444, Y500, Y730) in various combinations and/or in combination with incorporation of mutations that enhance tropism of the virus to a desired target cell or tissue type, and methods of generating the library. Such mutations can include insertion of one or more peptides for targeting the virus to a cell or tissue type.

In some aspects, the delivery vector is an rAAV vector having improved tropism to neuronal cells. In some aspects, the delivery vector is an rAAV vector having tropism to a glial cell or an astrocyte. Glial cells can be a cell of glial lineage or an activated glia. In other aspects, the delivery vector is an rAAV vector encapsidated in a capsid protein having tropism to activated astrocytes.

In some aspects, the programmable nucleic acid modification system is a CRISPR/Cas tool modified for transcriptional regulation of a locus. In some aspects, the programmable nucleic acid modification system is a CRISPR/Cas transcriptional regulator driven by cell-specific promoters using a catalytically dead effector (dCAS9) to modulate transcription of a chitinase gene encoding a chitinase protein.

The protein expression modification system can be encoded by a nucleic acid construct. Nucleic acid constructs can be as described in Section II herein below.

### II. Nucleic Acid Constructs

The protein expression modification system can be encoded by one or more nucleic acid constructs. The expression modification constructs generally comprise DNA coding sequences operably linked to at least one promoter control sequence for expression of the protein modification system in a target cell or tissue. Promotor control sequences can include constitutive, ubiquitous, regulated, cell- or tissue-specific promoters, or any combination thereof.

Suitable eukaryotic constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable eukaryotic regulated promoter control sequences include, without limit, those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, NphsI promoter, OG-2 promoter, SP-B promoter, SYN1 promoter, GFAP promoter and CBA promoter and WASP promoter. Promoter control sequences can also be promoter control sequences of the gene of interest, such that the expression pattern of the one or more nucleic acid constructs matches the expression pattern of the gene of interest. The promoter sequence can be wild type or it can be modified for more efficient or efficacious expression.

In some aspects, the promoter is an astrocyte-specific promoter. In some aspects, the promoter is a chimeric CMV-chicken β-actin promoter (CBA) promoter comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 87. In some aspects, the promoter is a chimeric CMV-chicken β-actin promoter (CBA) promoter comprising a nucleic acid sequence comprising about 75% or more, 85% or more, 95% or more, or 100% sequence identity with a sequence selected from SEQ ID NO: 87.

The nucleic acid constructs can comprise additional expression control sequences (e.g., enhancer sequences, Kozak sequences, polyadenylation sequences, transcriptional termination sequences, etc.), selectable reporter sequences (e.g., antibiotic resistance genes), origins of replication, and the like. The nucleic acid constructs can further comprise RNA processing elements such as glycine tRNAs or Csy4 recognition sites. Such RNA processing elements can, for instance, intersperse polynucleotide sequences encoding multiple gRNAs under the control of a single promoter to produce the multiple gRNAs from a transcript encoding the multiple gRNAs. When a cys4 recognition cite is used, a vector can further comprise sequences for expression of Csy4 RNAse to process the gRNA transcript. Additional information about nucleic acid constructs and use thereof may be found in “Current Protocols in Molecular Biology”, Ausubel et al., John Wiley & Sons, New York, 2003, or “Molecular Cloning: A Laboratory Manual”, Sambrook & Russell, Cold Spring Harbor Press, Cold Spring Harbor, NY, 3rd edition, 2001. Other methods of controlling expression in a specific tissue or target cell can be as described in Section I(c) and Section III.

Nucleic acid constructs encoding an expression modification system can comprise one or more constructs encoding the expression system. The nucleic acid constructs can be DNA or RNA, linear or circular, single-stranded or double-stranded, or any combination thereof. The nucleic acid constructs can be codon optimized for efficient translation into protein in the cell of interest. Codon optimization programs are available as freeware or from commercial sources.

### III. Methods

Another aspect of the present disclosure encompasses a method of treating a disease condition associated with expression of a chitinase protein in a cell or tissue type of a subject in need thereof. The method comprises modifying the expression of one or more chitinase proteins in the cell or tissue type in the subject by administering to the subject a therapeutically effective amount of a composition for modifying the expression of one or more chitinase proteins in a target cell or tissue type. The composition can be as described in Section I herein above.

It will be appreciated by those skilled in the art that a combination of more than one composition of the present disclosure may be used. It will also be appreciated by those skilled in the art that a composition of the present disclosure may be used in combination with other therapeutic agents before, after, and/or during treatment with a composition of the disclosure. Further, methods of the invention may be used in combination with standard treatments for a specific disorder.

### (a) Administering

The method comprises administering to the subject a therapeutically effective amount of a composition for modifying the expression of one or more chitinase proteins in a target cell or tissue type. A composition of the invention may be formulated for administration to a subject by several different means. For instance, a composition may generally be administered parenterally, intraperitoneally, intravascularly, transdermally, subcutaneously, rectally, or intrapulmonarily in dosage unit formulations containing conventional nontoxic pharmaceutically acceptable adjuvants, carriers, excipients, and vehicles as desired. The term parenteral as used herein includes subcutaneous, intravenous, intramuscular, intrathecal, or intrasternal injection, or infusion techniques. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

When the delivery vector is a viral vector, cells can be infected with the viral vector by contacting the cells with the vector. For instance, the cells can be tissue culture cells, and can be contacted with the viral vector by adding the vector to the cell culture. The cells can also be infected by delivering to a subject in compositions according to any appropriate methods known in the art. The viral vector, suspended in a physiologically compatible carrier (e.g., in a composition), may be administered to a subject. The subject can be a human, a livestock animal, a companion animal, a lab animal, or a zoological animal. In one aspect, the subject can be a rodent, e.g., a mouse, a rat, a guinea pig, etc. Non-limiting examples of suitable livestock animals can include pigs, cows, horses, goats, sheep, llamas and alpacas. Non-limiting examples of companion animals can include pets such as dogs, cats, rabbits, and birds. As used herein, a “zoological animal” refers to an animal that can be found in a zoo. Such animals can include non-human primates, large cats, wolves, and bears. Non-limiting examples of a laboratory animal can include rodents, canines, felines, and non-human primates. Non-limiting examples of rodents can include mice, rats, guinea pigs, etc. In some aspects, the subject is a human subject.

Delivery of the vector-mediated system and engineered vector compositions of the instant disclosure to a mammalian subject may be by, for example, intramuscular injection or by administration into the bloodstream of the mammalian subject. Administration into the bloodstream may be by injection into a vein, an artery, or any other vascular conduit. In some aspects, the compositions are administered into the bloodstream by way of isolated limb perfusion, a technique well known in the surgical arts, the method essentially enabling the artisan to isolate a limb from the systemic circulation prior to administration of the compositions. A variant of the isolated limb perfusion technique can also be employed by the skilled artisan to administer the compositions into the vasculature of an isolated limb to potentially enhance transduction into muscle cells or tissue. Moreover, in certain aspects, it may be desirable to deliver the compositions to the nervous system of a subject. Thus, the term includes, but is not limited to, neuronal cells, glial cells, astrocytes, cerebrospinal fluid (CSF), interstitial spaces, bone, cartilage, and the like. Compositions may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection.

Suitable carriers may be readily selected by one of skill in the art in view of the indication for which a delivery vector is directed. For example, one suitable carrier includes saline, which may be formulated with a variety of buffering solutions (e.g., phosphate buffered saline). Other carriers include sterile saline, lactose, sucrose, calcium phosphate, gelatin, dextran, agar, pectin, peanut oil, sesame oil, and water. The selection of the carrier is not a limitation of the disclosure.

Optionally, in addition to the compositions and carrier(s), other conventional pharmaceutical ingredients can be included, such as preservatives or chemical stabilizers. Suitable preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

Compositions are administered in sufficient amounts to introduce the nucleic acid construct into the cells of a desired tissue and to provide sufficient levels of expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

Formulation of pharmaceutically-acceptable excipients and carrier solutions is well known to those of skill in the art, as is the development of suitable dosing and treatment regimens for using the particular compositions described herein in a variety of treatment regimens. The amount of active compound in each therapeutically useful composition may be prepared is such a way that a suitable dosage is obtained in any given unit dose of the compound. Factors such as solubility, bioavailability, biological half-life, route of administration, product shelf life, as well as other pharmacological considerations, are contemplated by one skilled in the art of preparing such pharmaceutical formulations, and as such, a variety of dosages and treatment regimens may be desirable.

In certain aspects, when the delivery vector is a viral vector, it is desirable to deliver the compositions in suitably formulated pharmaceutical compositions disclosed herein either subcutaneously, intrapancreatically, intranasally, parenterally, intravenously, intramuscularly, intrathecally, or orally, intraperitoneally, or by inhalation. In some aspects, the cells are infected with the viral vectors by administering the vectors to a subject in a pharmaceutically-acceptable carrier to the subject in an amount and for a period of time sufficient to infect the cells. For instance, the viral vectors can be administered parenterally into the subject.

Pharmaceutical forms suitable for injectable use can include sterile aqueous solutions or dispersions and sterile powders for the extemporaneous preparation of sterile injectable solutions or dispersions. Dispersions may also be prepared in glycerol, liquid polyethylene glycols, and mixtures thereof, and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, isotonic agents can be included, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, sterile aqueous mediums that can be employed are known to those of skill in the art.

Sterile injectable solutions can be prepared by incorporating composition in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the other required ingredients from those enumerated above. In the case of sterile powders for the preparation of sterile injectable solutions, the methods of preparation can be vacuum-drying and freeze-drying techniques which yield a powder of the active ingredient, plus any additional desired ingredient from a previously sterile-filtered solution thereof.

Compositions can also be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions are administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles and the like, may be used for the introduction of the compositions of the disclosure into suitable host cells. In particular, the compositions may be formulated for delivery either encapsulated in a lipid particle, a liposome, a vesicle, a nanosphere, or a nanoparticle or the like.

In some aspects, the delivery vector of the engineered system is a rAAV vector. rAAV vectors can be as described in Section I(d) herein above.

Cell infection methods for infecting cells with the rAAVs are known. For instance, the cells can be infected with encapsidated rAAVs by contacting the cells with the encapsidated rAAVs. For instance, the cells can be tissue culture cells, and they can be contacted with the encapsidated rAAVs by adding the encapsidated rAAVs to the cell culture. The cells can also be infected by delivering to a subject in compositions according to any appropriate methods known in the art. The encapsidated rAAV, preferably suspended in a physiologically compatible carrier (e.g., in a composition), may be administered to a subject, e.g., host animal, such as a human, mouse, rat, cat, dog, sheep, rabbit, horse, cow, goat, pig, guinea pig, hamster, chicken, turkey, or a non-human primate (e.g., Macaque).

Delivery of the encapsidated rAAVs to a mammalian subject may be by, for example, intramuscular injection or by administration into the bloodstream of the mammalian subject. Administration into the bloodstream may be by injection into a vein, an artery, or any other vascular conduit. In some aspects, the encapsidated rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique can also be employed by the skilled artisan to administer the virions into the vasculature of an isolated limb to potentially enhance transduction into muscle cells or tissue. Moreover, in certain aspects, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the disclosure.

Optionally, in addition to the rAAV and carrier(s), other conventional pharmaceutical ingredients can be included, such as preservatives or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

In some aspects, rAAV compositions are formulated to reduce aggregation of AAV particles in the composition, particularly where high rAAV concentrations are present (e.g., ^{˜}1013 GC/ml or more). Methods for reducing aggregation of rAAVs are well known in the art and include, for example, addition of surfactants, pH adjustment, salt concentration adjustment, etc.

Formulation of pharmaceutically-acceptable excipients and carrier solutions is well known to those of skill in the art, as is the development of suitable dosing and treatment regimens for using the particular compositions described herein in a variety of treatment regimens. Typically, these formulations may contain at least about 0.1% of the active compound or more, although the percentage of the active ingredient(s) may, of course, be varied and may conveniently be between about 1% or 2% and about 70% or 80% or more of the weight or volume of the total formulation. Naturally, the amount of active compound in each therapeutically useful composition may be prepared in such a way that a suitable dosage is obtained in any given unit dose of the compound. Factors such as solubility, bioavailability, biological half-life, route of administration, product shelf life, as well as other pharmacological considerations, are contemplated by one skilled in the art of preparing such pharmaceutical formulations, and as such, a variety of dosages and treatment regimens may be desirable.

In certain aspects, it is desirable to deliver the rAAV-based therapeutic constructs in suitably formulated pharmaceutical compositions disclosed herein either subcutaneously, intrapancreatically, intranasally, parenterally, intravenously, intramuscularly, intrathecally, or orally, intraperitoneally, or by inhalation. In some aspects, the cells are infected with the rAAVs by administering the rAAVs to a subject in a pharmaceutically-acceptable carrier to the subject in an amount and for a period of time sufficient to infect the cells. For instance, the rAAVs can be administered parenterally into the subject. When the cells are neural cells, including microglial cells, the rAAVs can be administered by injection into the striatum.

### (b) Disease Conditions

The disease condition can be any disease condition associated with expression of one or more chitinase proteins in a cell or tissue type. In humans, chitinases and chitinase-like proteins function to modulate innate immune responses, extracellular tissue remodeling, fibrosis and solid carcinomas, cell migration and differentiation, and function to modulate inflammation in the progression of many human diseases. Accordingly, a disease condition can be any disease condition associated with defective innate immune responses, cell migration and differentiation, extracellular tissue remodeling, fibrosis and solid carcinomas, and inflammation.

In some aspects, the disease condition is a condition associated with inflammation. Disease conditions associated with inflammation are known in the art and include, for example, arthritic conditions including, but not limited to, rheumatoid arthritis, spondyloarthropathies, gouty arthritis, osteoarthritis, systemic lupus erythematosus, or juvenile arthritis, asthma, bronchitis, menstrual cramps, premature labor, tendinitis, bursitis, skin-related conditions such as psoriasis, eczema, burns, dermatitis, gastrointestinal conditions such as inflammatory bowel disease, Crohn's disease, gastritis, irritable bowel syndrome, or ulcerative colitis, vascular diseases such as migraine headaches, periarteritis nodosa, thyroiditis, aplastic anemia, Hodgkin's disease, sclerodoma, rheumatic fever, type I diabetes, neuromuscular junction disease including myasthenia gravis, white matter disease, multiple sclerosis, sarcoidosis, nephrotic syndrome, Behcet's syndrome, polymyositis, gingivitis, nephritis, hypersensitivity, allergic rhinitis, respiratory distress syndrome, endotoxin shock syndrome, atherosclerosis, cancers, conditions associated with pulmonary inflammation, such as that associated with viral infections or cystic fibrosis, neuroinflammatory diseases such as Alzheimer's disease (AD), stroke, traumatic brain injury (TBI), Parkinson's disease (PD), Amyotrophic lateral sclerosis (ALS), multiple sclerosis (MS), ataxia, Bell's palsy, or epilepsy.

In some aspects, the disease condition is cancer. In other aspects, the disease condition is an immune system condition.

One aspect of the present disclosure encompasses a method of treating a neurological condition. In some aspects, the condition is associated with expression of a chitinase protein in a cell or tissue type in the nervous system of a subject in need thereof. The method comprises modifying the expression of one or more chitinase proteins in the cell or tissue type in the nervous system of the subject by administering to the subject a therapeutically effective amount of a composition for modifying the expression of one or more chitinase proteins in a target cell or tissue type. The composition can be as described in Section I herein above.

In some aspects, the disease condition is a neurological disease condition. This invention relates to benz[b]azepine compounds useful in the treatment of neurological disorders generally in mammals such as man. More specifically, the compounds are useful in the treatment of strokes and/or other neurodegenerative disorders such as hypoglycemia, synucleinopathies, cerebral palsy, multiple system atrophies (MSAs) such as striatonigral degeneration, olivopontocerebellar ataxia, and Shy-Drager syndrome, transient cerebral ischemic attack, perinatal asphyxia, epilepsy, psychosis, Huntington's chorea, amyotrophic lateral sclerosis, Alzheimer's disease, Parkinson's disease, Olivo-pontocerebellar atrophy, vital-induced neurodegeneration such as in acquired immunodeficiency syndrome and its associated dementia, anoxia such as from drowning, spinal cord and brain trauma, poisoning by exogenous neurotoxins, and chronic pain, for the prevention of drug and alcohol withdrawal symptoms, and for the inhibition of tolerance and dependence to opiate analgesics.

In some aspects, the neurological condition is a neurodegenerative disease. Neurodegenerative diseases result from the deterioration of neurons, causing brain dysfunction. The diseases are loosely divided into two groups-conditions affecting memory that are ordinarily related to dementia, and conditions causing problems with movements. The most widely known neurodegenerative diseases include Alzheimer (or Alzheimer's) disease and its precursor mild cognitive impairment (MCI), Parkinson's disease (including Parkinson's disease dementia), and multiple sclerosis.

Less well-known neurodegenerative diseases include adrenoleukodystrophy, AIDS dementia complex, Alexander disease, Alper's disease, amyotrophic lateral sclerosis (ALS), ataxia telangiectasia, Batten disease, bovine spongiform encephalopathy, Canavan disease, cerebral amyloid angiopathy, cerebellar ataxia, Cockayne syndrome, corticobasal degeneration, Creutzfeldt-Jakob disease, diffuse myelinoclastic sclerosis, fatal familial insomnia, Fazio-Londe disease, Friedreich's ataxia, frontotemporal dementia or lobar degeneration, hereditary spastic paraplegia, Huntington disease, Kennedy's disease, Krabbe disease, Lewy body dementia, Lyme disease, Machado-Joseph disease, motor neuron disease, Multiple systems atrophy, neuroacanthocytosis, Niemann-Pick disease, Pelizaeus-Merzbacher Disease, Pick's disease, primary lateral sclerosis including its juvenile form, progressive bulbar palsy, progressive supranuclear palsy, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, Tabes dorsalis, Tay-Sachs disease, toxic encephalopathy, transmissible spongiform encephalopathy, Vascular dementia, and X-linked spinal muscular atrophy, as well as idiopathic or cryptogenic diseases as follows: synucleinopathy, progranulinopathy, tauopathy, amyloid disease, prion disease, protein aggregation disease, and movement disorder. A more comprehensive listing can be found at the web site (www) of the National Institute of Neurological Disorders and Stroke (ninds) of the National Institutes of Health (nih) of the United States government (gov) in a subdirectory (/disorder/disorder_index) web page (htm). It is understood that such diseases often go by more than one name and that a nosology can oversimplify pathologies that occur in combination or that are not archetypical. In some aspects, the neurological condition Alzheimer's disease (AD), Parkinson's disease (PD), Amyotrophic lateral sclerosis (ALS), multiple sclerosis (MS), ataxia, Bell's palsy, and epilepsy CNS infections and autoimmune disorders, mood disorders such as suicidality and violent suicide, schizophrenia, or any combination thereof.

Certain other disorders, such as postoperative cognitive dysfunction, have been described only recently, and they too can involve neurodegeneration. Other disorders such as epilepsy cannot be primarily neurodegenerative, but at some point in their progression, they might involve nerve degeneration.

In some aspects, the neurological disorder is a synucleinopathy. Synucleinopathies (also called α-Synucleinopathies) are neurodegenerative diseases characterized by the abnormal accumulation of aggregates of α-synuclein protein in neurons, nerve fibres or glial cells. There are three main types of synucleinopathy: Parkinson's disease (PD), dementia with Lewy bodies (DLB), and multiple system atrophy (MSA). Other rare disorders, such as various neuroaxonal dystrophies, also have α-synuclein pathologies. Additionally, autopsy studies have shown that around 6% of sporadic Alzheimer's Disease exhibit α-synuclein positive Lewy pathology, and are sub-classed as Alzheimer's Disease with Amygdalar Restricted Lewy Bodies (AD/ALB).

The protein expression modification system modifies the expression of one or more chitinase proteins in the nervous system. In some aspects, the expression modification system modifies the expression of one or more chitinase proteins to decrease the inflammatory profile of the one or more chitinase proteins in distinct glial subsets. In other aspects, the modification system modifies the expression of Chit-1, CHI3L1, CHI3L2, or any combination thereof, in the central nervous system. In some aspects, the neurological disorder is a synucleinopathy.

Another aspect of the present disclosure encompasses a method of treating amyotrophic lateral sclerosis (ALS) in a subject in need thereof. The method comprises modifying the expression of one or more chitinase proteins in a cell or tissue type associated with ALS in the nervous system of the subject by administering to the subject a therapeutically effective amount of a composition for modifying the expression of one or more chitinase proteins in a target cell or tissue type. The composition can be as described in Section I herein above. The protein expression modification system can modify the expression of one or more chitinase proteins in activated glial subtypes. The protein expression modification system can also modify the expression of Chit-1, CHI3L1, CHI3L2, or any combination thereof in the central nervous system. In some aspects, the protein expression modification system modifies the expression of Chit-1, CHI3L1, CHI3L2, or any combination thereof in a cell of glial lineage. In other aspects, the protein expression modification system reduces the expression of Chit-1 in activated microglia, reduces the expression of CHI3L1 in activated astrocytes, reduces the expression of CHI3L2 in activated microglia, or any combination thereof.

Yet another aspect of the present disclosure encompasses a method of treating PD in a subject in need thereof. The method comprises modifying the expression of one or more chitinase proteins in a cell or tissue type associated with PD in the nervous system of the subject by administering to the subject a therapeutically effective amount of a composition for modifying the expression of one or more chitinase proteins in a target cell or tissue type. The composition can be as described in Section I herein above.

It will be appreciated by those skilled in the art that a combination of more than one system of the present disclosure can be used. It will also be appreciated by those skilled in the art that a system of the present disclosure can be used in combination with other therapeutic agents before, after, and/or during treatment with a system of the disclosure. Further, methods of the invention can be used in combination with standard treatments for a specific disorder.

In some aspects, the neurological condition is multiple system atrophy (MSA). When the neurological condition is MSA, the protein expression modification system reduces the expression of CHI3L1 protein in activated astrocytes.

### IV. Kits

One aspect of the present disclosure encompasses a kit for modifying the expression of one or more chitinase proteins in a target cell or tissue type. The kit comprises one or more vector-mediated engineered systems for modifying the expression of the one or more chitinase proteins, one or more nucleic acid constructs encoding the vector-mediated engineered systems, an encapsidated rAAV vector comprising the nucleic acid constructs encoding the vector-mediated engineered systems, or any combination thereof. The one or more vector-mediated engineered systems can be as described in Section I. The one or more nucleic acid constructs can be as described in Section II. rAAV vectors comprising the nucleic acid constructs can be as described in Section I(d). Alternatively, the kit can comprise one or more cells comprising one or more engineered vector-mediated systems, the one or more nucleic acid constructs encoding the engineered vector-mediated system, the rAAV vector comprising the nucleic acid constructs, or any combination thereof.

As used herein, “kits” refer to a collection of elements including at least one non-standard laboratory reagent for use in the disclosed methods, in appropriate packaging, optionally containing instructions for use. A kit may further include any other components required to practice the methods, such as transfection reagents, cell growth media, selection media, in vitro transcription reagents, nucleic acid purification reagents, protein purification reagents, buffers, dry powders, concentrated solutions, or ready-to-use solutions, and the like. In some aspects, a kit comprises one or more containers that contain reagents for use in the methods. Containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

The instructions generally include information about the use of the kit in the disclosed methods. Instructions included in the kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

A “genetically modified” cell refers to a cell in which the nuclear, organellar or extrachromosomal nucleic acid sequences of a cell have been modified, i.e., the cell contains at least one nucleic acid sequence that has been engineered to contain an insertion of at least one nucleotide, a deletion of at least one nucleotide, and/or a substitution of at least one nucleotide.

As used herein, the term “tropism” can be used to describe a nucleic acid delivery vector wherein the delivery vector has an increased affinity to a specific cell or tissue type.

The terms “genome modification” and “genome editing” refer to processes by which a specific nucleic acid sequence in a genome is changed such that the nucleic acid sequence is modified. The nucleic acid sequence may be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence may be modified such that an altered product is made.

As used herein, the term “nervous system” refers to all cells and tissue of the peripheral nervous system and all cells and tissue of the central nervous system, including the brain and spinal cord of a vertebrate.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms may encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide may be linked by phosphodiester, phosphothioate, phosphoramidite, phosphorodiamidate bonds, or any combination thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the terms “target site”, “target sequence”, or “nucleic acid locus” refer to a nucleic acid sequence that defines a portion of a nucleic acid sequence to be modified or edited and to which a homologous recombination composition is engineered to target.

The terms “upstream” and “downstream” refer to locations in a nucleic acid sequence relative to a fixed position. Upstream refers to the region that is 5′ (i.e., near the 5′ end of the strand) to the position, and downstream refers to the region that is 3′ (i.e., near the 3′ end of the strand) to the position.

As used herein, the term “treating” refers to: (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures. In the context of a neurodegenerative disorder, “treat” and “treating” encompass alleviating, ameliorating, delaying the onset of, inhibiting the progression of, or reducing the severity of one or more symptoms associated with the neurodegenerative disorder.

As used herein, the term “subject” means that the subject is a mammal, such as a human, but can also be an animal, e.g., domestic animals (e.g., dogs, cats and the like), farm animals (e.g., cows, sheep, pigs, horses and the like), and laboratory animals (e.g., cynomolgus monkey, rats, mice, guinea pigs and the like).

As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

The term “a therapeutically effective amount” as used herein refers to an amount effective, at dosages, and for periods of time necessary, to achieve the desired result with respect to the treatment of a disease. For example, in the treatment of a neurodegenerative disease, an agent (i.e., a compound or a composition) which decreases, prevents, delays or suppresses or arrests any symptoms of the cancer would be effective. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two or more doses in a suitable form to be administered at one, two or more times throughout a designated time period. A therapeutically effective amount may be determined by the efficacy or potency of the particular composition, the disorder being treated, the duration or frequency of administration, the method of administration, and the size and condition of the subject, including that subject's particular treatment response. A therapeutically effective amount may be determined using methods known in the art, and may be determined experimentally, derived from therapeutically effective amounts determined in model animals such as the mouse, or a combination thereof. Additionally, the route of administration may be considered when determining the therapeutically effective amount. In determining therapeutically effective amounts, one skilled in the art may also consider the existence, nature, and extent of any adverse effects that accompany the administration of a particular compound in a particular subject.

As used herein, the term “gene” means a segment of DNA that contains all the information for the regulated biosynthesis of an RNA product, including promoters, exons, introns, and other untranslated regions that control expression.

As used herein, the term “locus” means a location on a chromosome or DNA molecule corresponding to a gene or a physical or phenotypic feature.

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Astrocytic CHI3L1 Expression is Elevated in the Brains of PD Patients

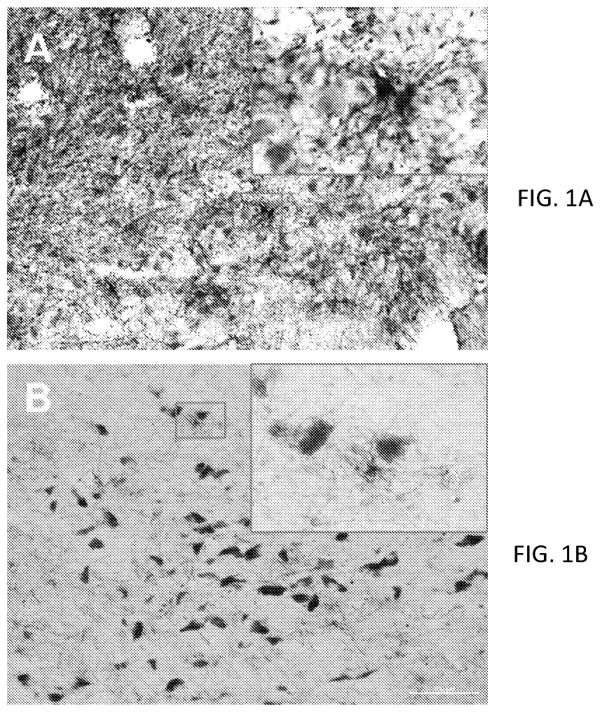

The inventors have shown an increase of astrocytic CHI3L1 in the AAV alpha-synuclein (α-syn) animal model-prior to neuronal loss. Data obtained by the inventors showed a significant increase in the number of midbrain astrocytes expressing CHI3L1 in PD brains as compared to age matched non-neurologic disease controls (FIG. 1). Particularly noticeable is increased cellular protein levels immediately adjacent to neuromelanin+ neurons in the SN. Accordingly, it is clear that CHI3L1 plays an important role in the activation of the immune response, and its dysregulation likely contributes to the pathogenesis of a number of neurological diseases.

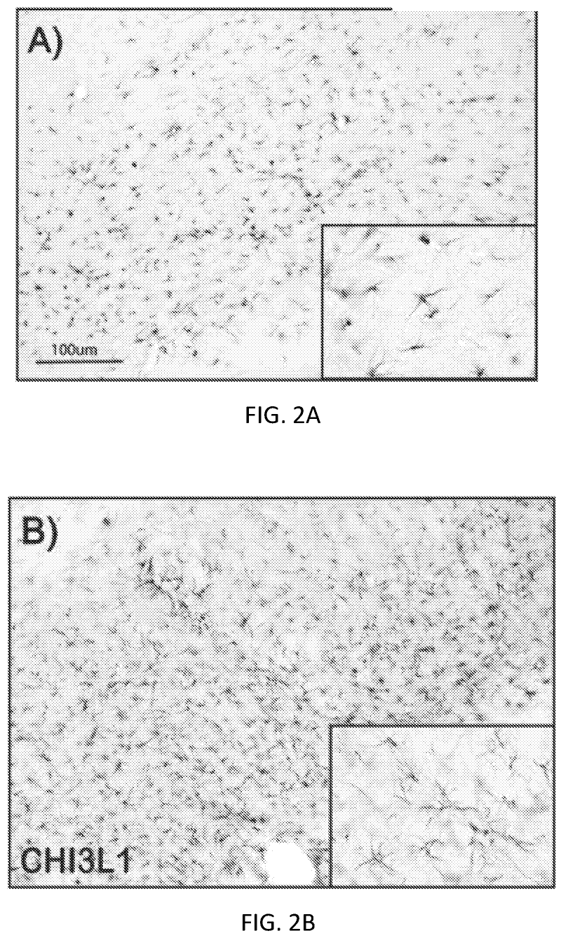

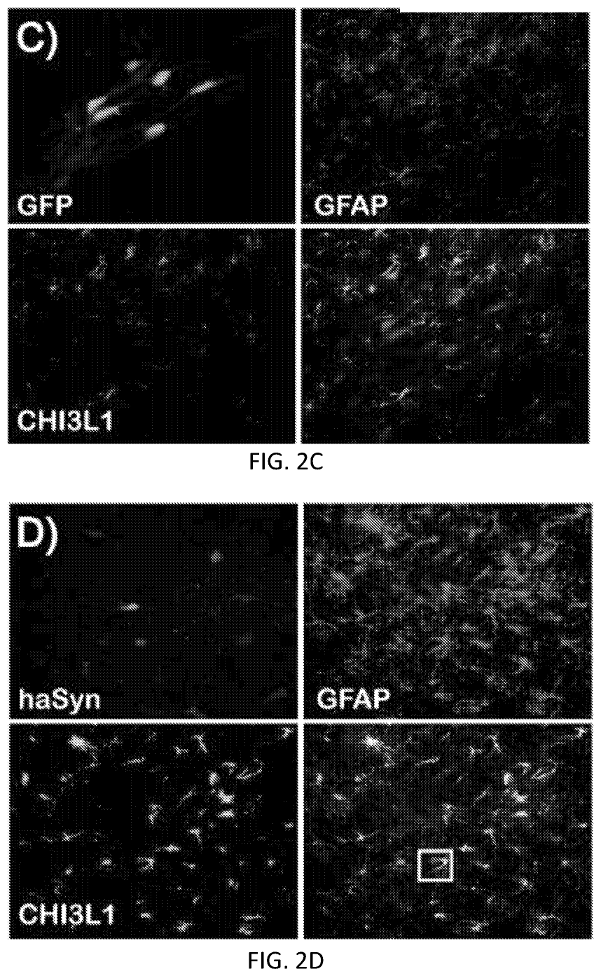

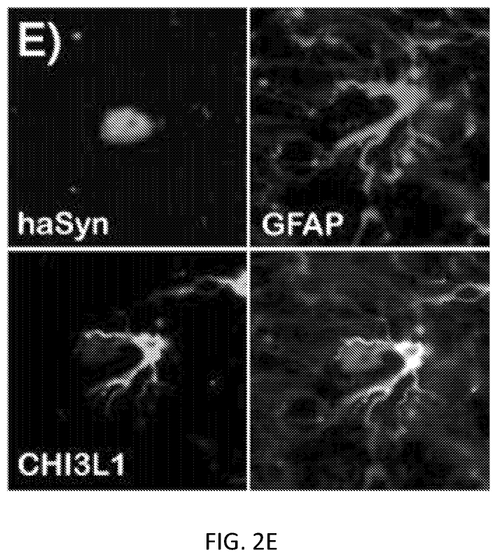

The inventors also showed that neuronal death in the AAV α-syn model of nigrostriatal degeneration is preceded by increased astrocytic CHI3L1 expression (FIG. 2), suggesting that CHI3L1 is an important mediator of detrimental neuroinflammation seen with α-syn toxicity.

### Example 2. Design and Engineering of AAV-CRISPR/Cas Transcriptional Regulators

In order to modulate CHI3L1 gene expression, a series of AAV genomes aimed at manipulating transcription of the endogenous CHI3L1 gene specifically in astrocytes is utilized. The inventors built a set of genomes utilizing a CRISPR/Cas tool modified for transcriptional regulation, using a catalytically dead effector (dCAS9) fused with a VP64 (transcriptional activator) or KRAB domain (transcriptional repressor) to either activate or repress transcription of the endogenous CHI3L1 gene, respectively. Specific guide RNAs (gRNAs), designed for the CHI3L1 promoter region, are tested for efficiency in rat astrocytes in vitro. The leading candidates (i.e. highest induction or suppression of CHI3L1) are cloned into the AAV genomes described above. Thus, control of endogenous expression is achieved using a single recombinant AAV. A similar vector with a gRNA designed against a non-targeted (LacZ) is used as a negative control. Vectors are evaluated in vivo for targeting specificity and efficiency of targeted transcriptional regulation. To this end, AAV vectors are injected into the midbrain using stereotaxic delivery. One month following vector delivery tissue is collected and analyzed for expression of Cas9 and CHI3L1.

### Example 3. AAV-Mediated Astrocytic Suppression of CHI3L1 in the AAV-α-Syn Rat Model of PD

The AAV-α-syn overexpression rat model of PD is used in this example. Targeted expression of human αSyn in DA neurons of the SN using AAV recapitulates many of the phenotypic pathological hallmarks observed in humans, including the accumulation of neuroinflammatory markers, α-syn protein aggregates, and progressive neuronal loss. It is well established that the amount of neurotoxicity produced by AAV-mediated α-syn overexpression is dose (i.e. virus titer) and time dependent. Based on this knowledge, a vector titer intended to produce modest neurodegeneration (˜50-60%) 3 months following vector delivery is used, facilitating detection of differences in neuronal vulnerability, in either direction, among the groups. Rats (n=12/group) receive a unilateral injection (2 sites×2 μl×2×10¹² vector genomes/mL) of AAV9-αSyn to the SN of young adult (3-month-old) rats. Simultaneously, AAV^{A}-CRISPR/Cas is injected to modulate expression of endogenous CHI3L1 as indicated in Table 1. The naïve contralateral hemisphere serves as an internal control, and an AAV-CRISPR with a LacZ gRNA is delivered to control animals. Animals are sacrificed at one month (ongoing DA neuronal loss), and 3 months (advanced neuronal loss).

TABLE 1
Experimental groups to determine effects of
CH13L expression in αSyn induced pathogenesis

| Exp. | rAAV2/9 group | Rationale |
|---|---|---|
| 2.2 | GFAP_SadCas9VP64- | Changes/toxicity associated with |
| | CH13L1 | increased CH13L expression |
| | GFAP_SadCas9VP64- | Negative control for CH13L |
| | control | overexpression |
| 2.3 | GFAP_SadCas9KRAB- | Changes/toxicity associated with |
| | CH13L1 | decreased CH13L expression |
| | GFAP_SadCas9KRAB- | Negative control for CH13L knock- |
| | control | down expression |

All groups will receive AAV-α-syn.
n = 12 rats/group

Animals in the 3-month survival group are subjected to monthly behavioral analysis indicative of dopamine loss (cylinder testing and amphetamine-induced rotations) to assess the integrity of the nigrostriatal system. Following sacrifice, series of quantitative histological measures are performed: 1) unbiased stereology analysis of TH immunoreactive neurons (THir) and HuC/D (pan-neuronal marker) is performed to quantify cell death and/or loss of phenotype; 2) Quantitative histology to validate CHI3L1 suppression; 3) Quantitative assessment of neuroinflammation (expression levels of Iba1, GFAP).

It is found that reducing expression of CHI3L1 ameliorates and exacerbates nigrostriatal neurodegeneration and neuroinflammatory indices, respectively.

### Example 4. AAV-Mediated Astrocytic Suppression of CHI3L1 in the AAV-α-Syn Rat Model of PD

AAV-mediated astrocytic overexpression of CHI3L1 in the AAV-α-syn rat model of PD is performed as described for Example 3, but utilizing AAV-CRISPR vectors designed to increase astrocytic expression of CHI3L1 (Table 1) and all outcomes are the same. Enhancing expression of CHI3L1 exacerbates nigrostriatal neurodegeneration and neuroinflammatory indices.

### Example 5. The Inflammatory Profile of Each Chitinase Gene in Distinct Glial Subsets in the Transgenic SOD1G93A Mouse Model of ALS

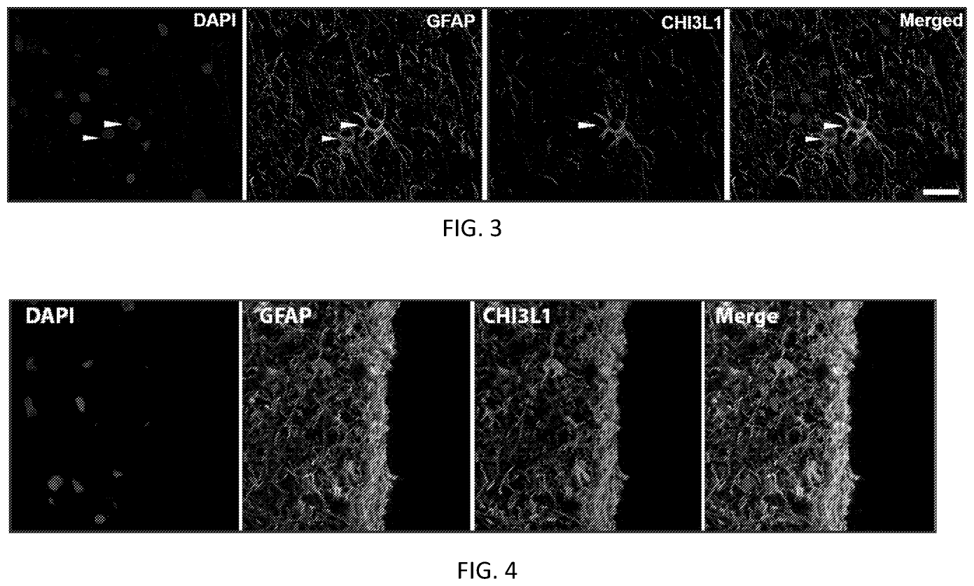

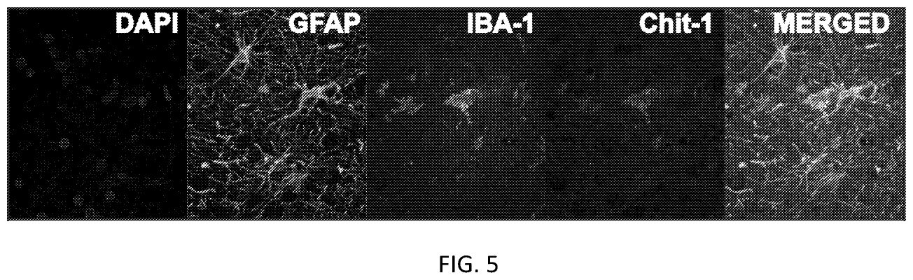

Using human post-mortem tissue sections from the Target ALS post-mortem tissue bank increased CHI3L1 expression as previously detected by the inventors in a subset of astrocytes located predominantly in the white matter of the motor cortex and spinal cord of ALS patients. CHI3L1 immunoreactivity was also increased in ALS patients in subpial layers of the cortex and surrounding some blood vessels in the gray matter. Confocal microscopy indicated that CHI3L1 was expressed in a subset of GFAP (glial fibrillary protein) positive reactive astrocytes (FIG. 3). Therefore, not all GFAP positive astrocytes express CHI3L1, and some CHI3L1 positive cells lack GFAP (data not shown). Within the subpial/layer 1 of the motor cortex, essentially all CHI3L1 positive cells were also GFAP immunoreactive (FIG. 4), suggesting that different astrocytes subtypes express CHI3L1 in different spatial locations. Chit-1 protein was detected in a subset of activated microglia and not GFAP positive astrocytes in ALS spinal cord (FIG. 5). The Chit-1 results confirm those previously reported in the spinal cord of ALS patients.

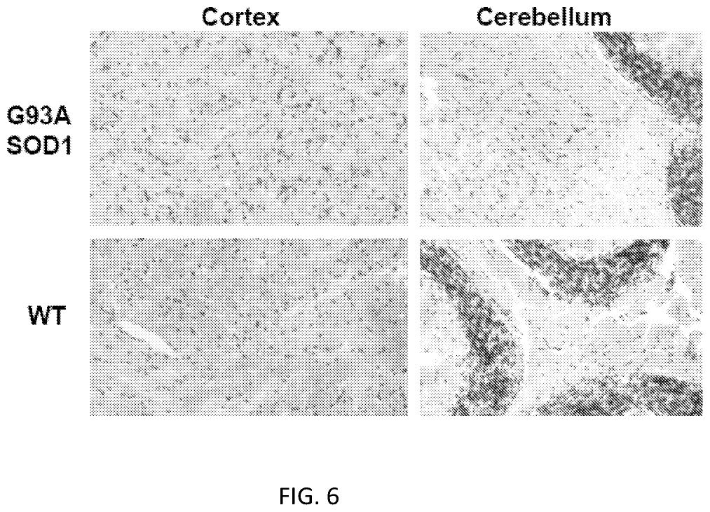

Ym1 expression is increased in cortex of transgenic G93A SOD1 mice prior to disease onset, correlating to an overall switch from neuroprotective to neurotoxic microglia. Preliminary studies were performed to explore BRP-39 expression in WT and transgenic SOD1 G93A mice, and similarly increased BRP-39 expression was noted within the cortex and cerebellum of mutant transgenic mice at the time of disease onset (FIG. 6).

### Example 6. Inflammatory Profile of Each Chitinase Gene in Distinct Glial Subsets in the Transgenic SOD1G93A Mouse Model of ALS

Adenoassociated virus (AAV) capsids paired with CRISPR/Cas technology are used to target specific glial subpopulations in the central nervous system to modulate endogenous chitinase gene expression in specific cell types. AAV is the leading vehicle for clinical gene therapy and the chief candidate for in vivo delivery of CRISPR/Cas therapeutic genome editing. Using an AAV based approach that targets specific cell types is superior to a global mouse knock-out approach. Moreover, using a CRISPR/Cas approach to modulate expression from an endogenous chitinase locus holds greater translation than approaches such as siRNA or overexpression using ectopic promoters.

Using a variety of directed evolution methods (e.g. capsid mutagenesis and target peptide insertion), novel AAV capsids with improved non-neuronal tropism were engineered. Combining these capsids with glial-specific promoters (e.g. truncated GFAP promoter for astrocytes, or the CD68 promoter for microglia) allows for a preponderance of transgene expression in the target cell per se. These engineered capsids are utilized to specifically target astrocytes or microglia to further define the spatial and glial subtypes targeted by these novel capsids. To modulate murine chitinase expression, a CRISPR/Cas tool modified for transcriptional regulation of the endogenous locus is utilized, using a catalytically dead effector (dCAS9) fused with a KRAB domain or a VP64 domain to either repress or activate transcription of the endogenous chitinase genes, respectively. Specific gRNAs for BRP-39, Ym1 or Chit-1 is carried within the same genome, thus, control of endogenous expression will be achieved using a single recombinant AAV genome.

Chitinase expression in specific glial subtypes results in either a pro-inflammatory (neurodegenerative) or anti-inflammatory (protective) condition (Table 2). More specifically, chitinase protein (Chit-1) promotes neuroinflammation while Chitinase-Like Proteins (CHI3L1 and CHI3L2) play a more anti-inflammatory neuroprotective role.

TABLE 2
Chitinase genes, cell type expression and contribution to disease.

| | | | Proposed | |
|---|---|---|---|---|
| Human | Mouse | Cell | Immune | |
| Gene | Homologue | Expression | function | References |
| Chit-1 | Chit-1 | Microglia | Pro- | [16, 21] |
| | | | inflammatory | |
| CH13L1 | BRP-39 | Astrocytes | Anti- | [10, 13, 19] |
| | | | inflammatory | |
| CH13L2 | Ym1 | Microglia | Anti- | [15, 22, 23] |
| | | | inflammatory | |

### Example 7. Chitinase Expression in Human Glial Sub-Populations

In this example, the type and location of glial populations that express Chit-1, CHI3L1 and CHI3L2 in ALS and control tissue sections are defined. Chit-1 co-localizes to a subset of microglia located predominately in white matter of the spinal cord and cortex, but also near neurons containing cytoplasmic TDP-43 pathology in the cortex and p62 pathology in the cerebellum. Chit-1 expressing microglia Iba-1+/MAC2+ in the cortex and TMEM119+ and CD74− in the white matter and spinal cord. These results are interpreted to be a more pro-inflammatory microglial response in ALS. CHI3L1 is be expressed in GFAP+/AGT+ astrocytes at the pial surface, but EAAT1+/AGT− astrocytes in both the white and gray matter, indicating a more pro-inflammatory phenotype at the pial surface but more anti-inflammatory in the deep white and gray matter.

CHI3L2 is expressed predominately in microglia in ALS and contained in Iba-1+/CD74+ microglia in the white matter and CX3CR1+/TREM2− defining microglia with more oligodendrogenesis/neurogenesis activity. Accordingly, CHI3L2 is a more anti-inflammatory and oligo- and neuroprotective activities in ALS.

Therefore, expression of the various chitinases occurs in a mixture of pro-inflammatory and alternatively activating, as well as anti-inflammatory (alternative activation—repair of cells and extracellular matrix) glial responses that are spatially and temporally dependent in the brain and spinal cord.

In line with the human studies, murine Chit-1 is shown to be expressed in Iba-1+/MAC2+ microglia in the cortex and spinal cord, as well as TMEM119+ microglia in the white matter. This represents Chit-1 expression in pro-inflammatory microglia responses throughout the CNS. BRP-39 expression is detected in EAAT1+/AGT-astrocytes in the cortex and GFAP+/AGT+ astrocytes in the spinal cord and pial surface of the cortex. Ym1 is observed in Iba-1+/CD74+ microglia in the white matter and CX3CR1+/TREM2−microglia in the gray matter, representing an alternative activation pathway of Ym1 positive microglia and an overall anti-inflammatory and repair/protective function. Ym1 and Chit-1 expression occur in overlapping or identical microglial subtypes.

TABLE 3
Markers used to define microglia and astrocyte subtypes.

| Markers of Microglia Subtypes | | Markers of Astrocyte Subtypes | |
|---|---|---|---|
| Iba-1+ | General marker of | GFAP+ | General marker of astrocyte activation |
| | microglia activation | | |
| CX3CR1+/TREM2− | Neuroprotective microglia | GFAP+/AGT+ | Activated A1 neurotoxic astrocytes at |
| | [49] | | pial surface and white matter [44, 45, |
| | | | 50] |
| Iba-1+/MAC2+ | Activated microglia | EAAT1+/AGT− | Activated A2 neuroprotective |
| | surrounding motor neurons | (GLAST) | astrocytes in gray matter expressing |
| | in ALS [51] | | glutamatergic neurotransmitter markers |
| | | | [44, 52] |
| TMEM119+ | Activated microglia | EAAT1−/AGT+ | Activated A3 astrocytes in gray and |
| | response to white matter | | white matter expressing GABAergic |
| | injury [41, 53] | | neurotransmitter (GAT3) markers [44, |
| | | | 50] |
| Iba-1+/CD74− | De-myelinating microglia | | |
| | [43] | | |
| Iba-1+/CD74+ | Re-myelinating microglia | | |
| | [43] | | |

The data presented herein shows a complex distribution, both spatially and in regard to phenotypic distribution, of the various chitinases in the ALS CNS, suggesting an apparent dichotomous role in disease. Within the human tissues, the relationship of chitinase expressing cells are correlated to ALS pathology (pTDP-43 or p62 inclusions) and clinical parameters of disease (duration of disease, site of disease onset, presence or absence of C9 repeat expansion).

Chit-1 co-localizes to a subset of microglia located predominately in white matter of the spinal cord and cortex, but also near neurons containing cytoplasmic TDP-43 pathology in the cortex and p62 pathology in the cerebellum. Chit-1 expressing microglia are Iba-1+/MAC2+ in the cortex and TMEM119+ and CD74−in the white matter and spinal cord. Therefore, Chit-1 can be a more pro-inflammatory microglial response in ALS (Table 4). CHI3L1 is expressed in GFAP+/AGT+ astrocytes at the pial surface, but EAAT1+/AGT− astrocytes in both the white and gray matter, indicating a more pro-inflammatory phenotype at the pial surface but more anti-inflammatory in the deep white and gray matter. CHI3L2 is expressed predominantly in microglia in ALS and contained in Iba-1+/CD74+ microglia in the white matter and CX3CR1+/TREM2-defining microglia with more oligodendrogenesis/neurogenesis activity. Therefore, CHI3L2 can be a more anti-inflammatory and oligo- and neuroprotective activities in ALS (Table 4).

TABLE 4
AAV-targeted approach to alter in vivo inflammatory condition

| Pro-inflammatory condition | Anti-inflammatory condition | Celltype |
|---|---|---|
| Increased Chit1 | Decreased Chit 1 | Microglia |
| Decreased BRP-39 | Increased BRP-39 | Astrocytes |
| Decreased Ym1 | Increased Ym1 | Microglia |

### Example 8. AAV-Based Animal Studies to Regulate Chitinase Gene Expression

As noted in the examples above, a dichotomous change in chitinase expression in specific glial subtypes results in either a pro-inflammatory (neurodegenerative) or anti-inflammatory (protective) condition (Table 4).

To modulate chitinase gene expression, a series of novel AAV genomes aimed at manipulating transcription of the endogenous chitinases: Chit-1, BRP-39 and Ym1 genes specifically in either microglia or astrocytes in the murine brain and spinal cord, are used. A set of genomes utilizing a CRISPR/Cas tool from Staphylococcus aureus modified for transcriptional regulation, using a catalytically dead effector (dCAS9) fused with a VP64 or KRAB domain to either activate or repress transcription of the endogenous genes, respectively were developed (see Table 5). Specific guide RNAs (gRNAs), designed for the promoter region of each gene (Chit-1, BRP-39 or Ym1) were designed using the Broad Institute Genetic Perturbation Platform webtool, and are tested for efficiency in cultured and LPS activated murine astrocytes (C8-D1A: ATCC CRL-2541) to stimulate expression of pro-inflamatory proteins or LPS activated murine microglia (C8-B4: ATCC CRL-2540) by quantifying changes in protein levels using immunoblots and mRNA using digital droplet PCR (ddPCR). The leading candidates (i.e. highest induction or repression of the respective proteins, with a minimum cut-off of 50% from baseline; see caveats below) is used to generate AAV. Thus, control of endogenous expression is achieved using a single recombinant AAV. A similar vector with a gRNA designed against a sequence not present in the mammalian genome (i.e LacZ) is used as a negative control.

TABLE 5
AAV genomes to modulate chitinase expression in glial subtypes

| Capsid | Promoter | Vector genome | Purpose |
|---|---|---|---|
| AAV^{A} | GFAP | dCas9-VP64-gBRP-39 | Enhance Chit1 expression in astrocytes |
| | | dCas9-KRAB-gBRP-39 | Repress Chit1 expression in astrocytes |
| | | dCas9-VP64-gLacZ | Control for VP64 activity in astrocytes |
| | | dCas9-KRAB-gLacZ | Control for KRAB activity in astrocytes |
| AAV^{M} | CD68 | dCas9-VP64-gYm1 | Enhance Ym1 expression in microglia |
| | | dCas9-KRAB-gYm1 | Repress Ym1 expression in microglia |
| | | dCas9-VP64-gChit1 | Enhance Chit1 expression in microglia |
| | | dCas9-KRAB-gChit1 | Repress Chit1 expression in microglia |
| | | dCas9-VP64-gLacZ | Control for VP64 activity in microglia |
| | | dCas9-KRAB-gLacZ | Control for KRAB activity in microglia |

Pathological studies using the designed AAV genomes show that increased expression of Chit-1 by microglia will increase pathology, increase disease progression, and reduce survival. Down regulation of anti-inflammatory murine chitinases (BRP-39 and Ym1) results in faster disease progression, reduced spinal cord motor neurons, and reduced survival of the SOD1 G93A mice. Similarly, increased expression of BRP-39 in astrocytes or Ym1 in microglia slows disease progression, reduces loss of motor neurons, and increases survival.

### Example 9. Distribution of Chitinase Expression in Synucleinopathies: Study Design and Results

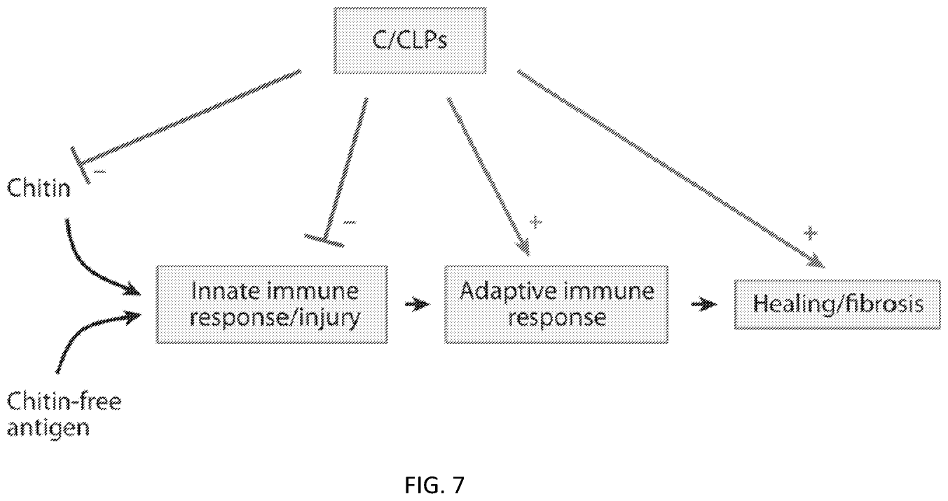

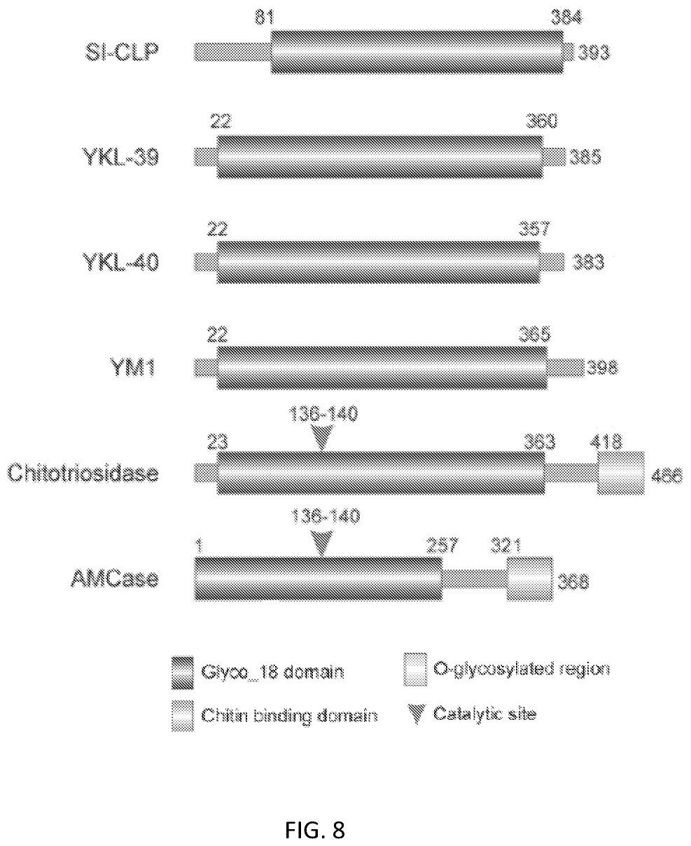

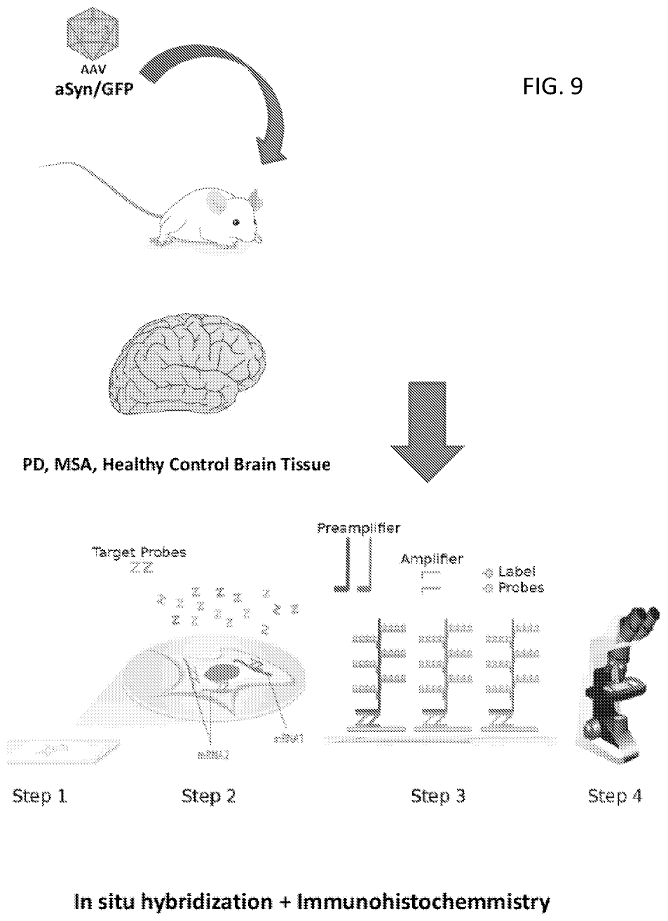

Chitinases and Chitinase-Like proteins (C/CLPs) belong to the glycoside hydrolase family 18. C/CLPs act as host-defense enzymes and are widely expressed in prokaryotes and eukaryotes and are believed to play a key role as regulators of the immune response, such of which are shown in FIG. 7. They are associated with many acute and chronic inflammatory conditions, including cancer and neurological disorders, such as Alzheimer's disease and amyotrophic lateral sclerosis. No studies exist in evaluating the expression of chitinases in synucleinopathies. The current study was to define the role of chitinases and chitinase-like proteins, such as those shown in FIG. 8, in the etiology of synucleinopathies and to develop therapeutic approaches to regulate their activity in order to halt or slow disease progression. It was hypothesized that astrocytic CHI3L1 expression is a histopathological feature associated with α-syn pathology in synucleinopathies, and that the regulation of astrocytic CHI3L1 may alter neuroinflammation in an α-syn overexpression models of synucleinopathies.

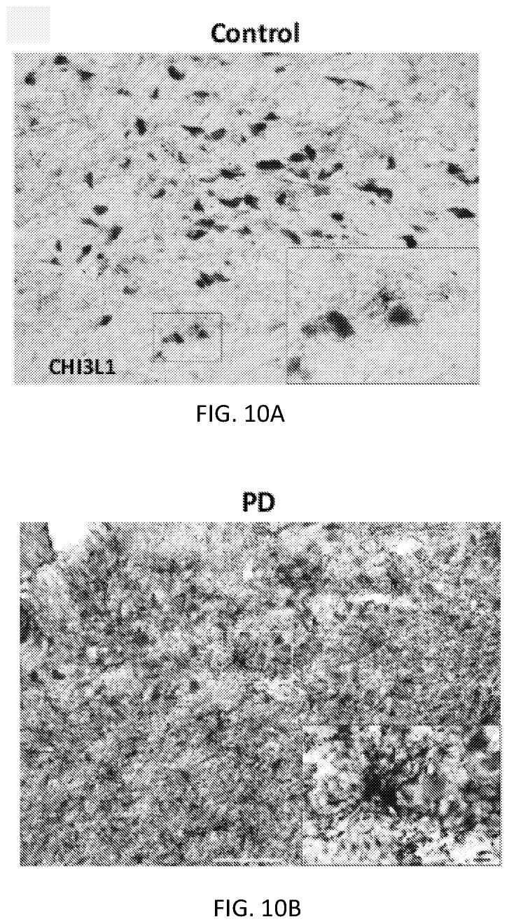

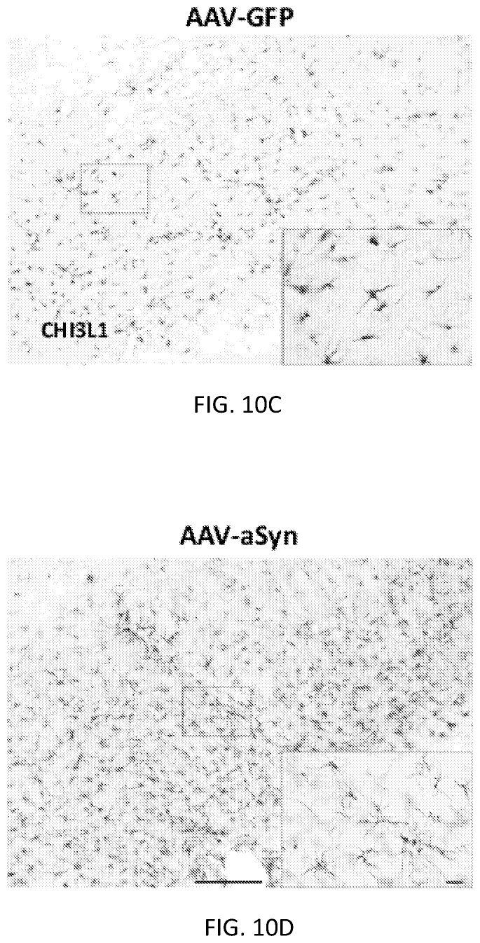

In the first part of the study, chitinase protein expression was identified and characterized in post-mortem brain sections from the substantia nigra (SN) of Healthy Control Subjects (FIG. 10A) and Parkinson's disease (PD) patients (FIG. 10B). These brain sections were stained for CHI3L1 (Chitinase 3 Like 1)/YKL-40. To model PD, rats were injected in the SN with AAV2/5 expressing either GFP (FIG. 10C) or human alpha-synuclein (FIG. 10D). To model multiple system atrophy (MSA), oligotrophic capsid Olig001 expressing either GFP (FIG. 10E) or human α-syn (FIG. 10F) was injected in the striatum of rats. Qualitatively higher expression patterns of CHI3L1 were observed in PD subjects (FIG. 10B), as well as both synucleinopathy animal models (FIG. 10D, FIG. 10E), when comparing to controls (FIG. 10A, FIG. 10C, FIG. 10E).

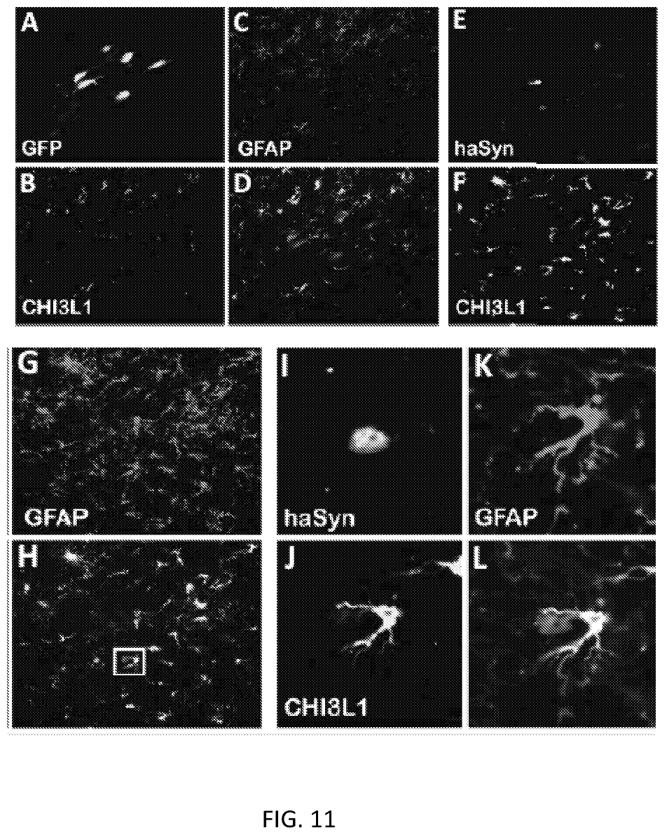

In the second part, CHI3L1 localization in astrocytes was studied. Rats were injected in the SN with either AAV-GFP (FIG. 11 panel A, FIG. 11 panel D) or AAV-αSyn (FIG. 11 panel E, FIG. 11 panel L). As shown in FIG. 11, histological analysis shows the colocalization of CHI3L1 with astrocyte marker GFAP in both AAV-GFP (FIG. 11 panel A, FIG. 11 panel D) and AAV− α-syn (FIG. 11 panel E, FIG. 11 panel L) injected animals. Moreover, the expression of CHI3L1 (FIG. 11 panel F) and GFAP (FIG. 11 panel G) appear to be increased in AAV-αSyn injected animals compared to AAV-GFP injected animals (FIG. 11 panel B, FIG. 11 panel C).

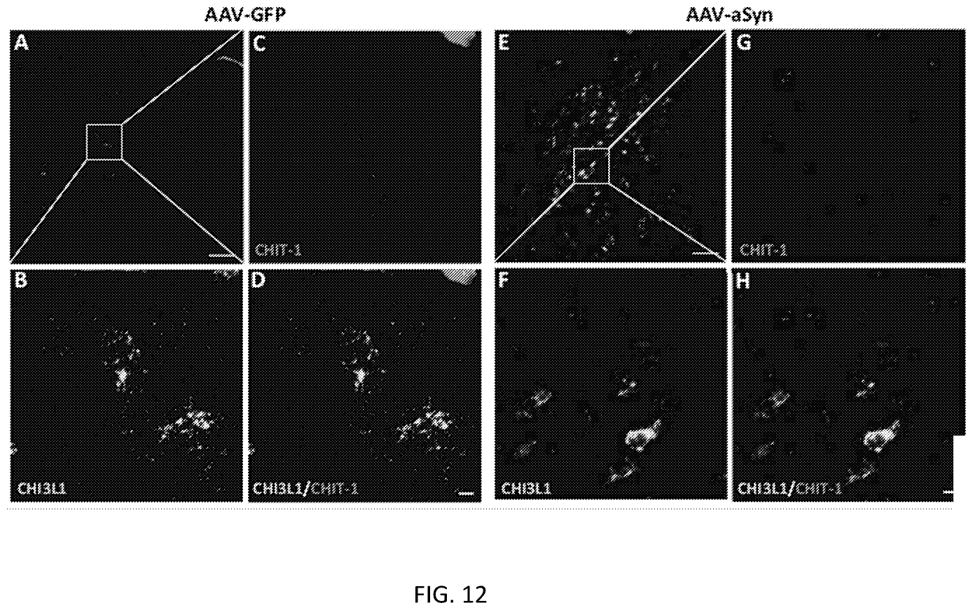

In the third part, in situ hybridization expression of CHI3L1 and CHIT-1 was studied. To evaluate the presence of Chitinase-Like protein and true chitinases, RNAscope™ probes were designed to target CHI3L1 and CHIT-1 respectively. As shown in FIG. 12, the pattern of CHI3L1 in situ hybridization (ISH) was more abundant than CHIT-1 in the SN of both GFP (FIG. 12 panel A-FIG. 12 panel D) and αSyn (FIG. 12 panel E-FIG. 12 panel H) injected rats. Moreover, CHI3L1 ISH was more abundant in αSyn (E-H) injected rats compared to GFP controls (FIG. 12 panel A-FIG. 12 panel D), validating the previous Immunohistochemistry results (FIG. 10). Scale Bar=100 μm in low magnification images (FIG. 12 panel A, FIG. 12 panel E) and 10 um in high magnification images (FIG. 12 panel B, FIG. 12 panel C, FIG. 12 panel D, FIG. 12 panel F, FIG. 12 panel G, FIG. 12 panel H).

The above studies showed that Chitinase 3 Like 1 (CHI3L1)/YKL-40 was increased in the SN of post-mortem PD subjects, as well as AAV-mediated αSyn overexpression models of PD and MSA, compared to control cases. RNAscope™ in situ hybridization showed apparent increase of CHI3L1, as opposed to true chitinase CHIT-1, in AAV-αSyn overexpression. Colocalization of CHI3L1 and GFAP highlighted astrocytes as a key player in the secretion of chitinases in synucleinopaties. Further studies include to characterize whether A1 (neurotoxic) or A2 (neuroprotective) reactive astrocytes are responsible for CHI3L1 secretion, and to modulate chitinase expression in rat α-syn over-expression models of PD and MSA.

### Example 10. Modulation of Chitinase Expression In Vivo

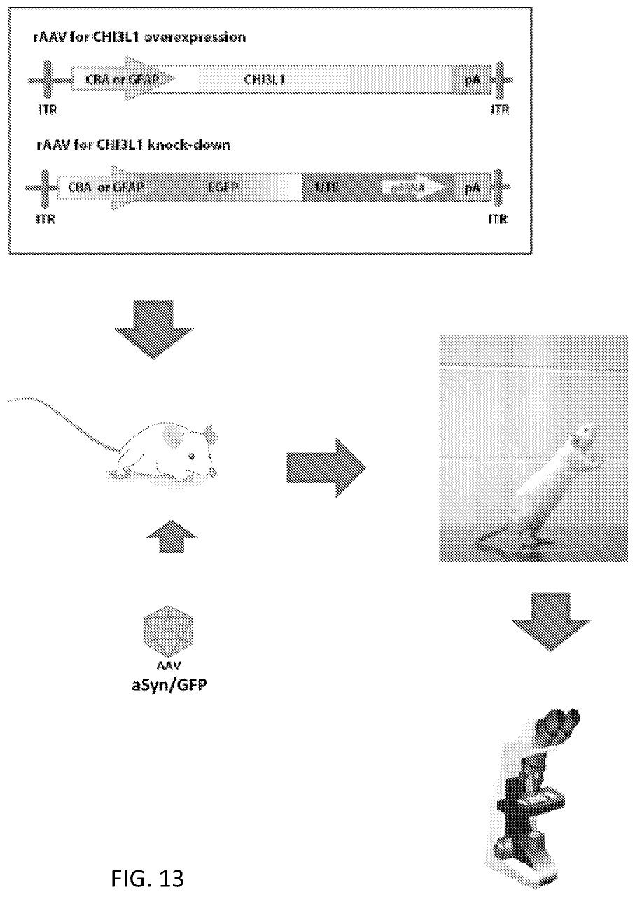

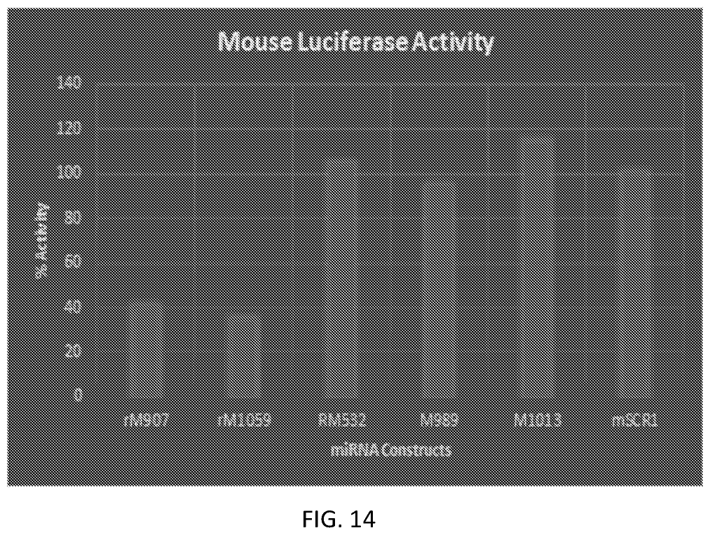

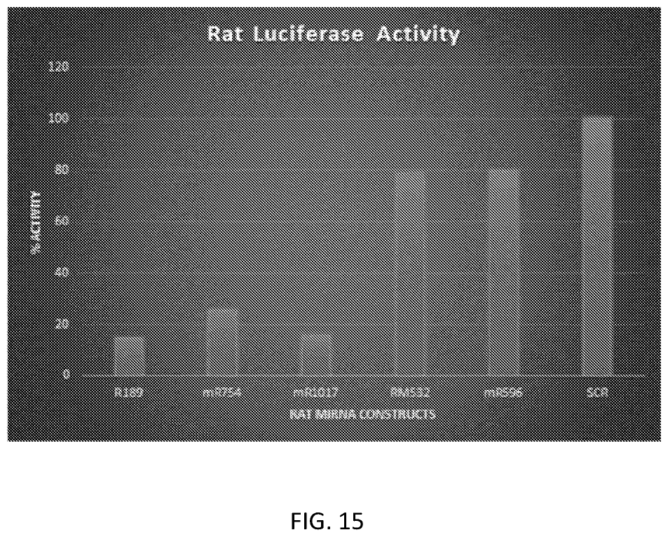

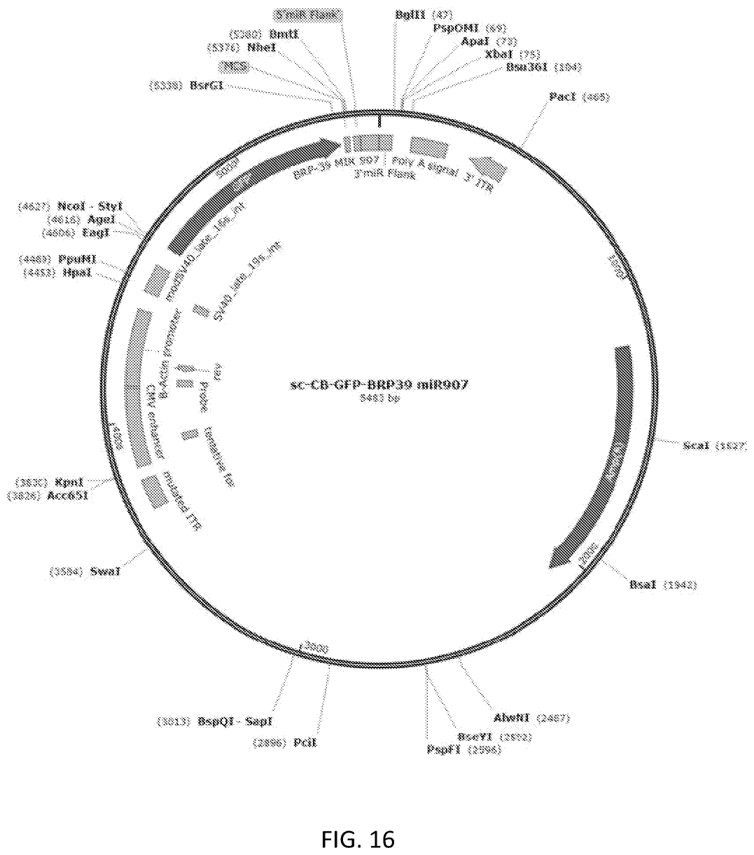

FIG. 13 provides the steps to modulate chitinase expression in rats, wherein rats were administered either with rAAV genomes comprising expression constructs for over-expression of CHI3L1 (FIG. 13 first panel; or rAAV genomes comprising expression constructs for CHI3L1 knock-down using miRNAs (FIG. 13 first panel). Similar design applied to mice studies. Various genomes were engineered for knocking down CHI3L1 expression in rat or mice using the rAAV genome of SEQ ID NO: 87. FIG. 16 shows a plasmid comprising the rAAV genome of SEQ ID NO: 87 having the miRNA of SEQ ID NO: 59. Knockdown of CHI3L1 using various miRNA sequences in mice and rats are shown in FIG. 14 (mice) and FIG. 15 (rats). Results show that the engineered genomes can knock down CHI3L1 expression in mice (FIG. 14) and rats (FIG. 15).

Other miRNAs were constructed to build plasmids for modulating chitinase expression (See sequence tables below).

Comparison of the cDNA sequences encoding mouse chitinase 3-like 1 (SEQ ID NO: 49) with the
cDNA sequences encoding rat chitinase 3-like 1 (SEQ ID NO: 64). Bold, underlined, italic,
and double underlined sequences denote miRNA target sequences.

| | | |
|---|---|---|
| BC005611.2:48-1193 | ATGGGCATGAGGGCGGCACTGACAGGCTTTGCGGTCCTGATGCTGCTCCAGAGCTGCTCT | 60 |
| BC091365.1:96-1208 | ---------------------------------------ATGCTGCTCCAGAGCTGCTCT | 21 |
| | ********************* | |
| BC005611.2:48-1193 | GCGTACAAGCTGGTCTGCTACTTCACCAGCTGGTCCCAGTACCGGGAAGGCGTTGGAAGC | 120 |
| BC091365.1:96-1208 | GCGTACAAACTGGTCTGCTACTACACCAACTGGTCCCAGTACCGGGAAGGCAATGGGAGC | 81 |
| | ******** ************* ***** ********************** *** *** | |
| BC005611.2:48-1193 | TTCTTACCAGACGCCATCCAACCTTTCCTGTGCACCCACATCATCTACAGCTTTGCCAAC | 180 |
| BC091365.1:96-1208 | TGCTTCCCAGATGCCCTCGACCATTCCCTGTGCACCCATATCATCTACAGCTTTGCCAAC | 141 |
| | * *** ***** *** ** * * ** ************ ********************* | |
| BC005611.2:48-1193 | ATCAGCAGCGACAACATGCTTAGCACATGGGAGTGGAATGACGAGTCGAACTATGACAAG | 240 |
| BC091365.1:96-1208 | ATCAGCAAC---AACAAGCTCAGCACATCGGAGTGGAATGACGTAACCCTGTATGGCATG | 198 |
| | ******* * **** *** ******* ************** * **** ** * | |
| BC005611.2:48-1193 | CTGAATAAACTGAAGACCAGAAACACCAACCTGAAGACCCTCCTGTCTGTTGGAGGGTGG | 300 |
| BC091365.1:96-1208 | CTGAATACTCTCAAGACCAGAAACCCCAGACTGAAGACACTGCTGTCTGTTGGAGGATGG | 258 |
| | ******* ** ************ *** ******** ** ************** *** | |
| BC005611.2:48-1193 | AAATTTGGCGAAAAAAGATTTTCCGAGATTGCCTCCAACACTGAGAGACGCACTGCTTTC | 360 |
| BC091365.1:96-1208 | AGCTTTGGCTCAGAAAGATTTTCCAGGATTGTCTCCAACGCTAAGAGTCGCAAGACTTTC | 318 |
| | * ****** * *********** ***** ******* ** **** **** ***** | |
| BC005611.2:48-1193 | GTCCGGTCGGTAGCCCCGTTCCTGCGTTCTTATGGCTTTGATGGGCTGGATCTCGCCTGG | 420 |
| BC091365.1:96-1208 | GTCCAGTCGGTAGCTCCCTTCCTGCGGACCTATGGCTTTGATGGACTGGATCTCGCCTGG | 378 |
| | **** ********* ** ******** * ************** *************** | |
| BC005611.2:48-1193 | CTCTACCCTCGCTTAAGAGACAAGCAGTATTTCTCCACCCTGATCAAGGAACTGAATGCG | 480 |
| BC091365.1:96-1208 | CTCTACCCGGGCCCGAAAGACAAGCAACATTTTACCACACTGATCAAGGAACTGAAGGCG | 438 |
| | ******** ** * ********* **** **** ***************** *** | |
| BC005611.2:48-1193 | GAATTCACAAAGGAGGTCCAGCCAGGCAGAGAGAAACTCCTGCTCAGCGCAGCTTTGTCA | 540 |
| BC091365.1:96-1208 | GAATTCACAAAGGAAGTCCAGCCAGGCACAGAGAAACTCCTGCTCAGTGCTGCCGTGTCA | 498 |
| | ************** ************* ****************** ** ** ***** | |
| BC005611.2:48-1193 | GCAGGAAAGGTGGCCATTGACACTGGCTATGACATCGCCCAGATAGCCCAACACCTGGAT | 600 |
| BC091365.1:96-1208 | GCAGGAAAGGTGACCCTTGACAGTGGCTATGATGTTGCCCAGATAGCCCAACACCTAGAT | 558 |
| | ************ ** ****** ********* * ******************** *** | |
| BC005611.2:48-1193 | TTTATCAATCTCATGACCTACGATTTCCATGGAGTCTGGCGCCAAATCACAGGCCACCAC | 660 |
| BC091365.1:96-1208 | TTCATTAATCTCATGACCTATGATTTCCATGGAACCTGGCGCCACACCACAGGACATCAC | 618 |
| | ** ** ************** ************ ********* * ****** ** *** | |
| BC005611.2:48-1193 | AGCCCCCTCTTCCAAGGCCAGAAGGACACTAGGTTTGACAGATACAGCAATGTGAACTAT | 720 |
| BC091365.1:96-1208 | AGCCCCCTCTTCCGAGGCCAGCAGGACACTGGGCCTGACAGATTCAGCAATGTGGACTAT | 678 |
| | ************* ******* ******** ** ******** ********** ***** | |
| BC005611.2:48-1193 | GCCGTGCAGTACATGATACGTCTGGGAGCCCAGGCCAGCAAGCTACTGATGGGCATCCCC | 780 |
| BC091365.1:96-1208 | GGTGTGGGGTACATGCTAAGGCTGGGAGCCCCCACCAACAAGCTAGTGATGGGTATCCCC | 738 |
| | * *** ******* ** * ********** *** ******* ******* ****** | |
| BC005611.2:48-1193 | ACCTTTGGGAAGAGCTTCACTCTGGCATCTTCTGAAAATCAGTTGGGAGCTCCAATCTCA | 840 |
| BC091365.1:96-1208 | ACCTTTGGAAAGAGCTTCACTCTGGCATCTTCTGAGAATCAAGTGGGAGCTCCAATCACA | 798 |
| | ******** ************************** ***** ************** ** | |
| BC005611.2:48-1193 | GGGGAAGGATTACCAGGCCGGTTCACCAAGGAGGCAGGGACCCTGGCCTACTACGAGATA | 900 |
| BC091365.1:96-1208 | GGGTCAGGATTACCAGGCCGCTACACCAAGGAGAAAGGGACCCTCGCCTACTACGAGATA | 858 |
| | *** *************** * ********** ********* *************** | |
| BC005611.2:48-1193 | TGCGACTTCCTCAAAGGAGCTGAAGTACATCGACTCTCCAACGAGAAGGTTCCCTTCGCT | 960 |
| BC091365.1:96-1208 | TGCGACTTCCTCAGAGGAGCTGAAGTACATAGAATTCTTGGCCAGCAGGTTCCCTTTGCT | 918 |
| | ************* **************** ** * * ** ********** *** | |
| BC005611.2:48-1193 | ACCAAGGGCAACCAGTGGGTGGGGTATGAGGACAAGGAGAGTGTCAAAAACAAGGTTGGG | 1020 |
| BC091365.1:96-1208 | ACCAAGGGCAACCAGTGGGTGGGGTATGATGACCCGGAGAGCGTCAAAAACAAGGTGAAG | 978 |
| | ***************************** *** ****** ************** * | |
| BC005611.2:48-1193 | TTCCTGAAGGAGAAGAAGCTGGCAGGAGCCATGGTGTGGGCACTGGATTTGGATGATTTC | 1080 |
| BC091365.1:96-1208 | TACCTGAAGAACAAGCAGCTGGCAGGAGCCATGGTGTGGGCAGTGGATTTGGATGATTTC | 1038 |
| | * ******* * *** ************************** ***************** | |
| BC005611.2:48-1193 | CAGGGCACCTGTCAGC---CGAAGGAATTCTTCCCGCTCACCAACGCCATCAAGGATGCC | 1137 |
| BC091365.1:96-1208 | CGGGGCTCCTTCTGTGGGCATAACGTACACTTCCCGCTCACCAACGCCATCAAGGAGGCC | 1098 |
| | * **** *** ** * * *************************** *** | |
| BC005611.2:48-1193 | CTGGCTTAG------ | 1146 |
| BC091365.1:96-1208 | CTGGCTGTGGCTTAG | 1113 |
| | ****** * | |

Comparison of the cDNA sequences encoding mouse chitinase 1 (chitotriosidase) (Chit1) (SEQ ID
NO: 17) with the cDNA sequences encoding rat chitinase 1 (chitotriosidase) (Chit1) (SEQ ID
NO: 33). Bold, underlined, italic, and double underlined sequences denote miRNA target
sequences.

| | | |
|---|---|---|
| NM_001284525.1:265-1659 | ATGGTGCAGTCCCTGGCCTGGGCAGGTGTGATGACTCTGCTGATGGTCCAGTGGGGCTCT | 60 |
| XM_032915045.1:585-1979 | ATGGTGCAGTCCCTGGCCTTGGCAGGTGTGATGACTCTGCTGATGATCCAGTGGGGCTCT | 60 |
| | ******************* ************************* ************** | |
| NM_001284525.1:265-1659 | GCTGCAAAACTGGTCTGCTACCTCACCAACTGGTCCCAGTACCGGACGGAGGCAGTTCGG | 120 |
| XM_032915045.1:585-1979 | GCTGCAAAACTGTTCTGCTACTTCACCAACTGGGCCCAGTACCGGTCCGGGGCAGCTCGA | 120 |
| | ************ ******** *********** *********** * * ***** *** | |
| NM_001284525.1:265-1659 | TTCTTTCCCAGGGATGTGGATCCCAACCTGTGTACCCACGTCATCTTTGCTTTTGCTGGA | 180 |
| XM_032915045.1:585-1979 | TTCCTACCTAGGGATGTGGATCCCAACCTGTGTACCCATGTCATCTATGCCTTTGCTGGA | 180 |
| | *** * ** ***************************** ******* *** ********* | |
| NM_001284525.1:265-1659 | ATGGACAACCATCAGCTCAGCACTGTGGAGCACAATGACGAACTTCTCTACCAGGAGCTG | 240 |
| XM_032915045.1:585-1979 | ATGAACAACCACCAGATCAGCACTGTAGAGCCCAATGACGAGCTTTTCTACCAAGAGCTG | 240 |
| | *** ******* *** ********** *** ********** *** ******* ****** | |
| NM_001284525.1:265-1659 | AACAGCCTAAAGACTAAGAACCCCAAGCTCAAGACCCTGTTAGCCGTTGGAGGCTGGACC | 300 |
| XM_032915045.1:585-1979 | AACAGCCTAAAGAAGAGGAACCCCAAGCTCAAGACCCTGTTAGCCGTCGGGGGCTGGAGC | 300 |
| | ************* * ****************************** ** ******* * | |
| NM_001284525.1:265-1659 | TTTGGTACCCAGAAGTTCACAGACATGGTGGCCACCGCCAGCAACCGGCAGACCTTTGTG | 360 |
| XM_032915045.1:585-1979 | TTTGGTACCCAGAAGTTCACAGACATGGTGGCCACAGCCAGCACCCGGCAGACCTTTGTC | 360 |
| | *********************************** ******* *************** | |
| NM_001284525.1:265-1659 | AAGTCAGCCCTAAGTTTCCTGCGCACTCAAGGTTTTGATGGCCTTGACCTTGACTGGGAG | 420 |
| XM_032915045.1:585-1979 | AACTCAGCTCTCTCGTTCCTGCGCACTCATGGTTTTGACGGCCTTGACCTTGACTGGGAA | 420 |
| | ** ***** ** ************** ******** ******************** | |
| NM_001284525.1:265-1659 | TTCCCAGGTGGACGTGGGAGCCCCACAGTAGACAAAGAGAGATTCACAGCCCTGATACAG | 480 |
| XM_032915045.1:585-1979 | TACCCAGGAAGCCGAGGGAGCCCAGCAGTAGACAAAGAGAGATTCACAGCGCTGATACAG | 480 |
| | * ****** * ** ******** ************************** ********* | |
| NM_001284525.1:265-1659 | GACTTGGCCAAAGCCTTCCAGGAGGAAGCCCAGTCCTCAGGGAAGGAACGCCTCCTTCTG | 540 |
| XM_032915045.1:585-1979 | GATTTGGCCAAAGCCTTCCAGGAGGAAGCCCGGGCCTCAGGGAAAAGTCGCCTCCTTCTG | 540 |
| | ** **************************** * ********** ************ | |
| NM_001284525.1:265-1659 | ACTGCAGCTGTACCGAGTGATCGAGGCCTGGTGGATGCTGGCTACGAGGTGGACAAGATT | 600 |
| XM_032915045.1:585-1979 | ACTGCAGCTGTACCAACTGGTCGAGGCCATGTGGATGCTGGTTATGAGGTGGACAAGATT | 600 |
| | ************** * ** ******** *********** ** *************** | |
| NM_001284525.1:265-1659 | GCCCAGAGCTTGGATTTCATCAACCTTATGGCCTACGACTTCCACAGCTCCTTGGAAAAG | 660 |
| XM_032915045.1:585-1979 | GCTCAGAGCTTGGATTTCATCAACCTTATGGCCTACGACTTCCACAGCTCCTGGGACAAG | 660 |
| | ** ************************************************* *** *** | |
| NM_001284525.1:265-1659 | ACCACAGGGCATAACAGCCCCCTCTACAAAAGGCAAGGAGAAAGTGGGGCAGCCGCTGAG | 720 |
| XM_032915045.1:585-1979 | ACCACAGGGCACAACAGCCCCCTCTACAAAAGGCAAGGAGAGACTGGGAAGGATGCTGAA | 720 |
| | *********** ***************************** * **** * ***** | |
| NM_001284525.1:265-1659 | CAAAACGTGGATGCTGCTGTGACGCTCTGGCTGCAGAAGGGGACCCCAGCCAGCAAACTG | 780 |
| XM_032915045.1:585-1979 | AAAAATGTGGATGCTGCTGTGACGCTCTGGCTGAAGAAGGGGACCCCTGCCAGCAAACTG | 780 |
| | **** *************************** ************* ************ | |
| NM_001284525.1:265-1659 | ATCCTTGGCATGCCTACCTATGGACGCTCTTTCACCTTGGCCTCCTCGTCAGACAATGGA | 840 |
| XM_032915045.1:585-1979 | ATGCTTGGCATGCCCGCCTACGGACGCTCCTTCACCCTGGCCTCCTCATCAGACAGTGGA | 840 |
| | ** *********** **** ******** ****** ********** ******* **** | |
| NM_001284525.1:265-1659 | GTTGGGGCCCCAGCCACAGGGCCTGGTGCCCCAGGCCCCTATACGAAGGACAAAGGGGTC | 900 |
| XM_032915045.1:585-1979 | GTTGGGGCCCCAGCCACAGGACCTGGGGCCCCAGGCCCCTATACTAAGGAAAAGGGGATA | 900 |
| | ******************** ***** ***************** ***** ** *** * | |
| NM_001284525.1:265-1659 | CTGGCTTACTATGAGGCCTGCTCCTGGAAGGAAAGACACAGAATCGAGGACCAGAAGGTG | 960 |
| XM_032915045.1:585-1979 | CTGGTTTACTTTGAGGTCTGCTCCTGGAAGGGAAAACAGAGAATCGAGGACCAGAAGGTG | 960 |
| | **** ***** ***** ************** ** *** ********************* | |
| NM_001284525.1:265-1659 | CCTTACGCCTTCCAGGACAACCAGTGGGTGAGCTTTGACGACGTGGAAAGCTTCAAAGCC | 1020 |
| XM_032915045.1:585-1979 | CCCTATGTCTCCCAGGGCAACCAATGGGTGGGATTTGATGACAGGGAAAGCTTCAGAGCC | 1020 |
| | ** ** * ** ***** ****** ****** * ***** *** *********** **** | |
| NM_001284525.1:265-1659 | AAGGCTGCCTACCTGAAACAGAAGGGGCTGGGAGGAGCCATGGTCTGGGTCCTGGACTTG | 1080 |
| XM_032915045.1:585-1979 | AAGGCTGCCTATGTGAAACAGAAGGGACTAGGAGGGGCCATGGTCTGGATCCTGGATGGG | 1080 |
| | *********** ************* ** ***** ************ ******* * | |
| NM_001284525.1:265-1659 | GATGACTTCAAGGGTTCCTTCTGCAACCAGGGCCCGTACCCTCTCATCCGGACACTACGG | 1140 |
| XM_032915045.1:585-1979 | GATGACTTCAAAGGTTCCTTCTGCAACGAGGGCCAGTACCCTCTCATCCGGACACTACAC | 1140 |
| | *********** *************** ****** *********************** | |
| NM_001284525.1:265-1659 | CAGGAACTAAATCTTCCATCCGAGACTCCAAGGAGCCCAGAACAGATAATACCTGAGCCA | 1200 |
| XM_032915045.1:585-1979 | CAGGAGCTGAGCCTTTCATCCGGGCCTCCAAGAAGCCCAGAACAGGAAGTACCTGGGCCA | 1200 |
| | ***** ** * *** ****** * ******* ************ * ****** **** | |
| NM_001284525.1:265-1659 | CGCCCATCTTCTATGCCAGAGCAGGGACCCAGCCCAGGGCTAGATAACTTCTGCCAAGGC | 1260 |
| XM_032915045.1:585-1979 | CACCAGCCTTCTGAGCCCGAGCAGGGCCCCAGCCCAGGGCTAGATAACTTCTGCCAAGGC | 1260 |
| | * ** ***** *** ******** ********************************* | |
| NM_001284525.1:265-1659 | AAAGCTGATGGGGTCTACCCCAACCCTGGAGACGAGTCCACTTACTACAACTGTGGAGGA | 1320 |
| XM_032915045.1:585-1979 | AAAGCTGATGGGCTCTACCCCAACCCTACAGAAAAGTCCAGTTTCTACAGCTGTGGAGGG | 1320 |
| | ************ ************** *** ****** ** ***** ********* | |
| NM_001284525.1: 265-1659 | GGGCGGCTGTTCCAGCAGAGCTGTCCTCCAGGCCTGGTGTTTAGAGCCTCTTGCAAATGT | 1380 |
| XM_032915045.1:585-1979 | GGGCGACTGTTCCAGCACAGCTGCCCCCCAGGCCTGGTGTTTATCGACTCTTGCAAATGC | 1380 |
| | ***** *********** ***** ** **************** * ************ | |
| NM_001284525.1:265-1659 | TGTACCTGGAGCTAA | 1395 |
| XM_032915045.1:585-1979 | TGTACCTGGGGTTGA | 1395 |
| | ********* * * * | |

### SEQUENCE LISTING

### SEQ ID NO: 1

Plasmid comprising an expression construct expressing rAAV genome comprising miRNA rm907 at nucleotides 5420-5483
LOCUS miR_entry_Plasmi 5419 bp DNA circular SYN 19 Oct. 2015
DEFINITION dsCB-GFP-mirFlank-ployA cut BsmBI..2435 to BsmBI..2395
ACCESSION miR_entry_Plasmi
KEYWORDS ⋅
SOURCE Unknown.
   ORGANISM Unknown
      Unclassified.
REFERENCE 1 (bases 1 to 5419)
   AUTHORS Self
   JOURNAL Unpublished.
COMMENT SECID/File created by Clone Manager, Scientific & Educational Software
FEATURES Location/Qualifiers

| | |
|---|---|
| misc_feature | 1..45 |
| | /dnas_title=″3′miR Flank″ |
| | /gene=″3′miR Flank″ |
| | /SECDrawAs=″Region″ |
| | /SECStyleId=″1″ |
| misc_feature | 114..240 |
| | /dnas_title=″Poly A signal″ |
| | /gene=″Poly A signal″ |
| | /product=″Rabbit globin poly A″ |
| | /SECDrawAs=″Region″ |
| | /SECStyleId=″1″ |
| misc_feature | complement (329..458) |
| | /dnas_title=″3′ ITR″ |
| | /gene=″3′ ITR″ |
| | /SECDrawAs=″Region″ |
| | /SECStyleId=″1″ |
| CDS | 1221..2078 |
| | /dnas_title=″Amp (R) ″ |
| | /gene=″Amp (R) ″ |
| | /SECDrawAs=″Gene″ |
| | /SECStyleId=″1″ |
| misc_feature | 3686..3791 |
| | /dnas_title=″mutated ITR″ |
| | /gene=″mutated ITR″ |
| | /SECDrawAs=″Region″ |
| | /SECStyleId=″1″ |
| misc_feature | 3838..4117 |
| | /dnas_title=″CMV enhancer″ |
| | /gene=″CMV enhancer″ |
| | /SECDrawAs=″Region″ |
| | /SECStyleId=″1″ |
| misc_feature | 3894..3923 |
| | /dnas_title=″tentative for″ |
| | /gene=″tentative for″ |
| | /SECDrawAs=″Region″ |
| | /SECStyleId=″1″ |
| misc_feature | 4124..4150 |
| | /dnas_title=″Probe″ |
| | /gene=″Probe″ |
| | /SECDrawAs=″Region″ |
| | /SECStyleId=″1″ |
| misc_feature | 4124..4393 |
| | /dnas_title=″B-Actin promoter″ |
| | /gene=″B-Actin promoter″ |
| | /product=″Chicken″ |
| | /SECDrawAs=″Region″ |
| | /SECStyleId=″1″ |
| misc_feature | complement (4192..4214) |
| | /dnas_title=″rev″ |
| | /gene=″rev″ |
| | /SECDrawAs=″Region″ |
| | /SECStyleId=″1″ |
| misc_feature | 4459..4489 |
| | /dnas_title=″SV40_late_19s_int″ |
| | /gene=″SV40_late_19s_int″ |
| | /SECDrawAs=″Region″ |
| | /SECStyleId=″1″ |
| misc_feature | 4459..4555 |
| | /dnas_title=″modSV40_late_16s_int″ |
| | /gene=″modSV40_late_16s_int″ |
| | /SECDrawAs=″Region″ |
| | /SECStyleId=″1″ |
| CDS | 4629..5349 |
| | /dnas_title=″GFP ′ ″ |
| | /gene=″GFP ′ ″ |
| | /SECDrawAs=″Gene″ |
| | /SECStyleId=″1″ |
| misc_feature | 5365..5381 |
| | /dnas_title=″ ′MCS″ |
| | /gene=″ ′MCS″ |
| | /SECDrawAs=″Region″ |
| | /SECStyleId=″1″ |
| misc_feature | 5396..5419 |
| | /dnas_title=″5′miR Flank′ ″ |
| | /gene=″5′miR Flank′ ″ |
| | /SECDrawAs=″Region″ |
| | /SECStyleId=″1″ |
| source | 1..5419 |
| | /dnas_title=″sc-CB-GFP-BRP39 miR907″ |
| misc_feature | 5420..5483 |
| | /note=″BRP-39 MIR 907″ |

| | |
|---|---|
| 1 | caggacacaa ggcctgttac tagcactcac atggaacaaa tggcccagat ctggccgcac |
| 61 | tcgaaaacgg gccctctaga ctcgaggacg gggtgaacta cgcctgagga tccgatcttt |
| 121 | ttccctctgc caaaaattat ggggacatca tgaagcccct tgagcatctg acttctggct |
| 181 | aataaaggaa atttattttc attgcaatag tgtgttggaa ttttttgtgt ctctcactcg |
| 241 | gaagcaattc gttgatctga atttcgacca cccataatac ccattaccct ggtagataag |
| 301 | tagcatggcg ggttaatcat taactacaag gaacccctag tgatggagtt ggccactccc |
| 361 | tctctgcgcg ctcgctcgct cactgaggcc gggcgaccaa aggtcgcccg acgcccgggc |
| 421 | tttgcccggg cggcctcagt gagcgagcga gcgcgcagcc ttaattaacc taattcactg |
| 481 | gccgtcgttt tacaacgtcg tgactgggaa aaccctggcg ttacccaact taatcgcctt |
| 541 | gcagcacatc cccctttcgc cagctggcgt aatagcgaag aggcccgcac cgatcgccct |
| 601 | tcccaacagt tgcgcagcct gaatggcgaa tgggacgcgc cctgtagcgg cgcattaagc |
| 661 | gcggcgggtg tggtggttac gcgcagcgtg accgctacac ttgccagcgc cctagcgccc |
| 721 | gctcctttcg ctttcttccc ttcctttctc gccacgttcg ccggctttcc ccgtcaagct |
| 781 | ctaaatcggg ggctcccttt agggttccga tttagtgctt tacggcacct cgaccccaaa |
| 841 | aaacttgatt agggtgatgg ttcacgtagt gggccatcgc cctgatagac ggtttttcgc |
| 901 | cctttgacgt tggagtccac gttctttaat agtggactct tgttccaaac tggaacaaca |
| 961 | ctcaacccta tctcggtcta ttcttttgat ttataaggga ttttgccgat ttcggcctat |
| 1021 | tggttaaaaa atgagctgat ttaacaaaaa tttaacgcga attttaacaa aatattaacg |
| 1081 | cttacaattt aggtggcact tttcggggaa atgtgcgcgg aacccctatt tgtttatttt |
| 1141 | tctaaataca ttcaaatatg tatccgctca tgagacaata accctgataa atgcttcaat |
| 1201 | aatattgaaa aaggaagagt atgagtattc aacatttccg tgtcgccctt attccctttt |
| 1261 | ttgcggcatt ttgccttcct gtttttgctc acccagaaac gctggtgaaa gtaaaagatg |
| 1321 | ctgaagatca gttgggtgca cgagtgggtt acatcgaact ggatctcaac agcggtaaga |
| 1381 | tccttgagag ttttcgcccc gaagaacgtt ttccaatgat gagcactttt aaagttctgc |
| 1441 | tatgtggcgc ggtattatcc cgtattgacg ccgggcaaga gcaactcggt cgccgcatac |
| 1501 | actattctca gaatgacttg gttgagtact caccagtcac agaaaagcat cttacggatg |
| 1561 | gcatgacagt aagagaatta tgcagtgctg ccataaccat gagtgataac actgcggcca |
| 1621 | acttacttct gacaacgatc ggaggaccga aggagctaac cgcttttttg cacaacatgg |
| 1681 | gggatcatgt aactcgcctt gatcgttggg aaccggagct gaatgaagcc ataccaaacg |
| 1741 | acgagcgtga caccacgatg cctgtagcaa tggcaacaac gttgcgcaaa ctattaactg |
| 1801 | gcgaactact tactctagct tcccggcaac aattaataga ctggatggag gcggataaag |
| 1861 | ttgcaggacc acttctgcgc tcggcccttc cggctggctg gtttattgct gataaatctg |
| 1921 | gagccggtga gcgtgggtct cgcggtatca ttgcagcact ggggccagat ggtaagccct |
| 1981 | cccgtatcgt agttatctac acgacgggga gtcaggcaac tatggatgaa cgaaatagac |
| 2041 | agatcgctga gataggtgcc tcactgatta agcattggta actgtcagac caagtttact |
| 2101 | catatatact ttagattgat ttaaaacttc atttttaatt taaaaggatc taggtgaaga |
| 2161 | tcctttttga taatctcatg accaaaatcc cttaacgtga gttttcgttc cactgagcgt |
| 2221 | cagaccccgt agaaaagatc aaaggatctt cttgagatcc tttttttctg cgcgtaatct |
| 2281 | gctgcttgca aacaaaaaaa ccaccgctac cagcggtggt ttgtttgccg gatcaagagc |
| 2341 | taccaactct ttttccgaag gtaactggct tcagcagagc gcagatacca aatactgttc |
| 2401 | ttctagtgta gccgtagtta ggccaccact tcaagaactc tgtagcaccg cctacatacc |
| 2461 | tcgctctgct aatcctgtta ccagtggctg ctgccagtgg cgataagtcg tgtcttaccg |
| 2521 | ggttggactc aagacgatag ttaccggata aggcgcagcg gtcgggctga acggggggtt |
| 2581 | cgtgcacaca gcccagcttg gagcgaacga cctacaccga actgagatac ctacagcgtg |
| 2641 | agctatgaga aagcgccacg cttcccgaag ggagaaaggc ggacaggtat ccggtaagcg |
| 2701 | gcagggtcgg aacaggagag cgcacgaggg agcttccagg gggaaacgcc tggtatcttt |
| 2761 | atagtcctgt cgggtttcgc cacctctgac ttgagcgtcg atttttgtga tgctcgtcag |
| 2821 | gggggcggag cctatggaaa aacgccagca acgcggcctt tttacggttc ctggcctttt |
| 2881 | gctggccttt tgctcacatg ttctttcctg cgttatcccc tgattctgtg gataaccgta |
| 2941 | ttaccgcctt tgagtgagct gataccgctc gccgcagccg aacgaccgag cgcagcgagt |
| 3001 | cagtgagcga ggaagcggaa gagcgcccaa tacgcaaacc gcctctcccc gcgcgttggc |
| 3061 | cgattcatta atgcagctga ttctaacgag gaaagcacgt tatacgtgct cgtcaaagca |
| 3121 | accatagtac gcgccctgta gcggcgcatt aagcgcggcg ggtgtggtgg ttacgcgcag |
| 3181 | cgtgaccgct acacttgcca gcgccctagc gcccgctcct ttcgctttct tcccttcctt |
| 3241 | tctcgccacg ttcgccggct ttccccgtca agctctaaat cgggggctcc ctttagggtt |
| 3301 | ccgatttagt gctttacggc acctcgaccc caaaaaactt gattagggtg atggttcacg |
| 3361 | tagtgggcca tcgccctgat agacggtttt tcgccctttg acgttggagt ccacgttctt |
| 3421 | taatagtgga ctcttgttcc aaactggaac aacactcaac cctatctcgg tctattcttt |
| 3481 | tgatttataa gggattttgc cgatttcggc ctattggtta aaaaatgagc tgatttaaca |
| 3541 | aaaatttaac gcgaatttta acaaaatatt aacgcttaca atttaaatat ttgcttatac |
| 3601 | aatcttcctg tttttggggc ttttctgatt atcaaccggg gtacatatga ttgacatgct |
| 3661 | agttttacga ttaccgttca tcgccctgcg cgctcgctcg ctcactgagg ccgcccgggc |
| 3721 | aaagcccggg cgtcgggcga cctttggtcg cccggcctca gtgagcgagc gagcgcgcag |
| 3781 | agagggagtg gaattcacgc gtggatctga attcaattca cgcgtggtac ctctggtcgt |
| 3841 | tacataactt acggtaaatg gcccgcctgg ctgaccgccc aacgaccccc gcccattgac |
| 3901 | gtcaataatg acgtatgttc ccatagtaac gccaataggg actttccatt gacgtcaatg |
| 3961 | ggtggagtat ttacggtaaa ctgcccactt ggcagtacat caagtgtatc atatgccaag |
| 4021 | tacgccccct attgacgtca atgacggtaa atggcccgcc tggcattatg cccagtacat |
| 4081 | gaccttatgg gactttccta cttggcagta catctactcg aggccacgtt ctgcttcact |
| 4141 | ctccccatct cccccccctc cccaccccca attttgtatt tatttatttt ttaattattt |
| 4201 | tgtgcagcga tgggggcggg gggggggggg gggcgcgcgc caggcggggc ggggcggggc |
| 4261 | gaggggcggg gcggggcgag gcggagaggt gcggcggcag ccaatcagag cggcgcgctc |
| 4321 | cgaaagtttc cttttatggc gaggcggcgg cggcggcggc cctataaaaa gcgaagcgcg |
| 4381 | cggcgggcgg gagcgggatc agccaccgcg gtggcggcct agagtcgacg aggaactgaa |
| 4441 | aaaccagaaa gttaactggt aagtttagtc tttttgtctt ttatttcagg tcccggatcc |
| 4501 | ggtggtggtg caaatcaaag aactgctcct cagtggatgt tgcctttact tctaggcctg |
| 4561 | tacggaagtg ttacttctgc tctaaaagct gcggaattgt acccgcggcc gatccaccgg |
| 4621 | tcgccaccat ggtgagcaag ggcgaggagc tgttcaccgg ggtggtgccc atcctggtcg |
| 4681 | agctggacgg cgacgtaaac ggccacaagt tcagcgtgtc cggcgagggc gagggcgatg |
| 4741 | ccacctacgg caagctgacc ctgaagttca tctgcaccac cggcaagctg cccgtgccct |
| 4801 | ggcccaccct cgtgaccacc ctgacctacg gcgtgcagtg cttcagccgc taccccgacc |
| 4861 | acatgaagca gcacgacttc ttcaagtccg ccatgcccga aggctacgtc caggagcgca |
| 4921 | ccatcttctt caaggacgac ggcaactaca agacccgcgc cgaggtgaag ttcgagggcg |
| 4981 | acaccctggt gaaccgcatc gagctgaagg gcatcgactt caaggaggac ggcaacatcc |
| 5041 | tggggcacaa gctggagtac aactacaaca gccacaacgt ctatatcatg gccgacaagc |
| 5101 | agaagaacgg catcaaggtg aacttcaaga tccgccacaa catcgaggac ggcagcgtgc |
| 5161 | agctcgccga ccactaccag cagaacaccc ccatcggcga cggccccgtg ctgctgcccg |
| 5221 | acaaccacta cctgagcacc cagtccgccc tgagcaaaga ccccaacgag aagcgcgatc |
| 5281 | acatggtcct gctggagttc gtgaccgccg ccgggatcac tctcggcatg gacgagctgt |
| 5341 | acaagtaaag cggccctagc gtttccggcg acggtgctag cgtcgaccag tggatcctgg |
| 5401 | aggcttgctg aaggctgtaT GCTGTACTTC AGCTCCTTTG AGGAAGTTTT GGCCACTGAC |
| 5461 | TGACTTCCTC AAGAGCTGAA GTA |

//

| SEQ | | |
|---|---|---|
| ID | | |
| NO. | Sequence | Description |
| 2 | ATGGCCAAGCTCATTCTTGTCACAGGTCTGGCAATTCTTCTGAACGTACAGCTGGGA | Coding sequence |
| | TCTTCCTACCAGCTGATGTGCTACTATACCAGTTGGGCTAAGGACAGGCCAATAGAA | of Mus musculus |
| | GGGAGTTTCAAACCTGGTAATATTGACCCCTGCCTGTGTACTCACCTGATCTATGCC | secretory |
| | TTTGCTGGAATGCAGAATAATGAGATCACTTACACACATGAGCAAGACTTGCGTGAC | protein |
| | TATGAAGCATTGAATGGTCTGAAAGACAAGAACACTGAGCTAAAAACTCTCCTGGCC | precursor |
| | ATTGGAGGATGGAAGTTTGGACCTGCCCCGTTCAGTGCCATGGTCTCTACTCCTCAG | (Ym1). |
| | AACCGTCAGATATTCATTCAGTCAGTTATCAGATTCCTTCGTCAATATAACTTTGAT | GenBank: |
| | GGCCTCAACCTGGACTGGCAGTACCCTGGGTCTCGAGGAAGCCCTCCTAAGGACAAA | M94584.2: 13- |
| | CATCTCTTCAGTGTTCTGGTGAAGGAAATGCGTAAAGCTTTTGAGGAAGAATCTGTG | 1209 |
| | GAGAAAGACATTCCAAGGCTGCTACTCACTTCCACAGGAGCAGGAATCATTGACGTA | |
| | ATCAAGTCTGGGTACAAGATCCCTGAACTGTCTCAGTCTCTTGACTATATTCAGGTC | |
| | ATGACATATGATCTCCATGATCCTAAGGATGGCTACACTGGAGAAAATAGTCCCCTC | |
| | TATAAATCTCCATATGACATTGGAAAGAGTGCTGATCTCAATGTGGATTCAATCATT | |
| | TCCTACTGGAAGGACCATGGAGCAGCTTCTGAGAAGCTCATTGTGGGATTTCCAGCA | |
| | TATGGGCATACCTTTATCCTGAGTGACCCTTCTAAGACTGGAATTGGTGCCCCTACA | |
| | ATTAGTACTGGCCCACCAGGAAAGTACACAGATGAATCAGGACTCCTGGCTTACTAT | |
| | GAGGTTTGTACATTTCTGAATGAAGGAGCCACTGAGGTCTGGGATGCCCCCCAGGAA | |
| | GTACCCTATGCCTATCAGGGTAATGAGTGGGTTGGTTATGACAATGTCAGGAGCTTC | |
| | AAGTTGAAGGCTCAGTGGCTCAAGGACAACAATTTAGGAGGTGCCGTGGTCTGGCCC | |
| | CTGGACATGGATGACTTCAGTGGTTCTTTCTGTCACCAGAGACATTTCCCTCTGACA | |
| | TCTACTTTAAAGGGAGATCTCAATATACACAGTGCAAGTTGCAAGGGCCCTTATTGA | |
| 3 | ACCAGCTGATGTGCTACTATA | Target sequence |
| | | for miRNA in |
| | | mouse Ym1 |
| | | sequence. |
| 4 | TGCTGTATAGTAGCACATCAGCTGGTGTTTTGGCCACTGACTGACACCAGCTGGTGC | MicroRNA |
| | TACTATA | sequence |
| | | targeting SEQ |
| | | ID NO: 3 |
| 5 | CCTGTGTACTCACCTGATCTA | Target sequence |
| | | for miRNA in |
| | | mouse Ym1 |
| | | sequence. |
| 6 | TGCTGTAGATCAGGTGAGTACACAGGGTTTTGGCCACTGACTGACCCTGTGTACACC | MicroRNA |
| | TGATCTA | sequence |
| | | targeting SEQ |
| | | ID NO: 5 |
| 7 | CAAGACTTGCGTGACTATGAA | Target sequence |
| | | for miRNA in |
| | | mouse Ym1 |
| | | sequence. |
| 8 | TCTCTTCAGTGTTCTGGTGAA | Target sequence |
| | | for miRNA in |
| | | mouse Ym1 |
| | | sequence. |
| 9 | AGCAGGAATCATTGACGTAAT | Target sequence |
| | | for miRNA in |
| | | mouse Ym1 |
| | | sequence. |
| 10 | TATCCTGAGTGACCCTTCTAA | Target sequence |
| | | for miRNA in |
| | | mouse Ym1 |
| | | sequence. |
| 11 | TGCTGTTAGAAGGGTCACTCAGGATAGTTTTGGCCACTGACTGACTATCCTGAGACC | MicroRNA |
| | CTTCTAA | sequence |
| | | targeting SEQ |
| | | ID NO: 10 |
| 12 | TGAATCAGGACTCCTGGCTTA | Target sequence |
| | | for miRNA in |
| | | mouse Ym1 |
| | | sequence. |
| 13 | ATGTCAGGAGCTTCAAGTTGA | Target sequence |
| | | for miRNA in |
| | | mouse Ym1 |
| | | sequence. |
| 14 | TGCTGTCAACTTGAAGCTCCTGACATGTTTTGGCCACTGACTGACATGTCAGGCTTC | MicroRNA |
| | AAGTTGA | sequence |
| | | targeting SEQ |
| | | ID NO: 14 |
| 15 | GGCTCAAGGACAACAATTTAG | Target sequence |
| | | for miRNA in |
| | | mouse Ym1 |
| | | sequence. |
| 16 | TTCCCTCTGACATCTACTTTA | Target sequence |
| | | for miRNA in |
| | | mouse Ym1 |
| | | sequence. |
| 17 | ATGGTGCAGTCCCTGGCCTGGGCAGGTGTGATGACTCTGCTGATGGTCCAGTGGGGC | NM 001284525.1: |
| | TCTGCTGCAAAACTGGTCTGCTACCTCACCAACTGGTCCCAGTACCGGACGGAGGCA | 265-1659 Mus |
| | GTTCGGTTCTTTCCCAGGGATGTGGATCCCAACCTGTGTACCCACGTCATCTTTGCT | musculus |
| | TTTGCTGGAATGGACAACCATCAGCTCAGCACTGTGGAGCACAATGACGAACTTCTC | chitinase 1 |
| | TACCAGGAGCTGAACAGCCTAAAGACTAAGAACCCCAAGCTCAAGACCCTGTTAGCC | (chitotriosidase) |
| | GTTGGAGGCTGGACCTTTGGTACCCAGAAGTTCACAGACATGGTGGCCACCGCCAGC | (Chit1), |
| | AACCGGCAGACCTTTGTGAAGTCAGCCCTAAGTTTCCTGCGCACTCAAGGTTTTGAT | transcript |
| | GGCCTTGACCTTGACTGGGAGTTCCCAGGTGGACGTGGGAGCCCCACAGTAGACAAA | variant 1, mRNA |
| | GAGAGATTCACAGCCCTGATACAGGACTTGGCCAAAGCCTTCCAGGAGGAAGCCCAG | |
| | TCCTCAGGGAAGGAACGCCTCCTTCTGACTGCAGCTGTACCGAGTGATCGAGGCCTG | |
| | GTGGATGCTGGCTACGAGGTGGACAAGATTGCCCAGAGCTTGGATTTCATCAACCTT | |
| | ATGGCCTACGACTTCCACAGCTCCTTGGAAAAGACCACAGGGCATAACAGCCCCCTC | |
| | TACAAAAGGCAAGGAGAAAGTGGGGCAGCCGCTGAGCAAAACGTGGATGCTGCTGTG | |
| | ACGCTCTGGCTGCAGAAGGGGACCCCAGCCAGCAAACTGATCCTTGGCATGCCTACC | |
| | TATGGACGCTCTTTCACCTTGGCCTCCTCGTCAGACAATGGAGTTGGGGCCCCAGCC | |
| | ACAGGGCCTGGTGCCCCAGGCCCCTATACGAAGGACAAAGGGGTCCTGGCTTACTAT | |
| | GAGGCCTGCTCCTGGAAGGAAAGACACAGAATCGAGGACCAGAAGGTGCCTTACGCC | |
| | TTCCAGGACAACCAGTGGGTGAGCTTTGACGACGTGGAAAGCTTCAAAGCCAAGGCT | |
| | GCCTACCTGAAACAGAAGGGGCTGGGAGGAGCCATGGTCTGGGTCCTGGACTTGGAT | |
| | GACTTCAAGGGTTCCTTCTGCAACCAGGGCCCGTACCCTCTCATCCGGACACTACGG | |
| | CAGGAACTAAATCTTCCATCCGAGACTCCAAGGAGCCCAGAACAGATAATACCTGAG | |
| | CCACGCCCATCTTCTATGCCAGAGCAGGGACCCAGCCCAGGGCTAGATAACTTCTGC | |
| | CAAGGCAAAGCTGATGGGGTCTACCCCAACCCTGGAGACGAGTCCACTTACTACAAC | |
| | TGTGGAGGAGGGCGGCTGTTCCAGCAGAGCTGTCCTCCAGGCCTGGTGTTTAGAGCC | |
| | TCTTGCAAATGTTGTACCTGGAGCTAA | |
| 18 | GCACAATGACGAACTTCTCTA | Target sequence |
| | | for miRNA in |
| | | mouse Chit1 |
| | | sequence. |
| 19 | ACTTCTCTACCAGGAGCTGAA | Target sequence |
| | | for miRNA in |
| | | mouse Chit1 |
| | | sequence. |
| 20 | TGCTGTTCAGCTCCTGGTAGAGAAGTGTTTTGGCCACTGACTGACACTTCTCTCAGG | MicroRNA |
| | AGCTGAA | sequence |
| | | targeting SEQ |
| | | ID NO: 19 |
| 21 | CCTTTGTGAAGTCAGCCCTAA | Target sequence |
| | | for miRNA in |
| | | mouse Chit1 |
| | | sequence. |
| 22 | TTTGTGAAGTCAGCCCTAAGT | Target sequence |
| | | for miRNA in |
| | | mouse Chit1 |
| | | sequence. |
| 23 | TGCTGACTTAGGGCTGACTTCACAAAGTTTTGGCCACTGACTGACTTTGTGAACAGC | MicroRNA |
| | CCTAAGT | sequence |
| | | targeting SEQ |
| | | ID NO: 22 |
| 24 | CCTAAGTTTCCTGCGCACTCA | Target sequence |
| | | for miRNA in |
| | | mouse Chit1 |
| | | sequence. |
| 25 | TGCTGTACAGCTGCAGTCAGAAGGAGGTTTTGGCCACTGACTGACCTCCTTCTCTGC | MicroRNA |
| | AGCTGTA | sequence |
| | | targeting SEQ |
| | | ID NO: 24 |
| 26 | GAGAGATTCACAGCCCTGATA | Target sequence |
| | | for miRNA in |
| | | mouse Chit1 |
| | | sequence |
| 27 | CTCCTTCTGACTGCAGCTGTA | Target sequence |
| | | for miRNA in |
| | | mouse Chit1 |
| | | sequence |
| 28 | GCTTGGATTTCATCAACCTTA | Target sequence |
| | | for miRNA in |
| | | mouse Chit1 |
| | | sequence. |
| 29 | TGCTGTAAGGTTGATGAAATCCAAGCGTTTTGGCCACTGACTGACGCTTGGATCATC | MicroRNA |
| | AACCTTA | sequence |
| | | targeting SEQ |
| | | ID NO: 28 |
| 30 | TGAGCTTTGACGACGTGGAAA | Target sequence |
| | | for miRNA in |
| | | mouse Chit1 |
| | | sequence. |
| 31 | TGCTGTTTCCACGTCGTCAAAGCTCAGTTTTGGCCACTGACTGACTGAGCTTTCGAC | MicroRNA |
| | GTGGAAA | sequence |
| | | targeting SEQ |
| | | ID NO: 30 |
| 32 | GTGTTTAGAGCCTCTTGCAAA | Target sequence |
| | | for miRNA in |
| | | mouse Chit1 |
| | | sequence |
| 33 | ATGGTGCAGTCCCTGGCCTTGGCAGGTGTGATGACTCTGCTGATGATCCAGTGGGGC | XM_032915045.1: |
| | TCTGCTGCAAAACTGTTCTGCTACTTCACCAACTGGGCCCAGTACCGGTCCGGGGCA | 585-1979 |
| | GCTCGATTCCTACCTAGGGATGTGGATCCCAACCTGTGTACCCATGTCATCTATGCC | PREDICTED: |
| | TTTGCTGGAATGAACAACCACCAGATCAGCACTGTAGAGCCCAATGACGAGCTTTTC | Rattus rattus |
| | TACCAAGAGCTGAACAGCCTAAAGAAGAGGAACCCCAAGCTCAAGACCCTGTTAGCC | chitinase |
| | GTCGGGGGCTGGAGCTTTGGTACCCAGAAGTTCACAGACATGGTGGCCACAGCCAGC | (Chit1), |
| | ACCCGGCAGACCTTTGTCAACTCAGCTCTCTCGTTCCTGCGCACTCATGGTTTTGAC | transcript |
| | GGCCTTGACCTTGACTGGGAATACCCAGGAAGCCGAGGGAGCCCAGCAGTAGACAAA | variant X2, |
| | GAGAGATTCACAGCGCTGATACAGGATTTGGCCAAAGCCTTCCAGGAGGAAGCCCGG | mRNA |
| | GCCTCAGGGAAAAGTCGCCTCCTTCTGACTGCAGCTGTACCAACTGGTCGAGGCCAT | |
| | GTGGATGCTGGTTATGAGGTGGACAAGATTGCTCAGAGCTTGGATTTCATCAACCTT | |
| | ATGGCCTACGACTTCCACAGCTCCTGGGACAAGACCACAGGGCACAACAGCCCCCTC | |
| | TACAAAAGGCAAGGAGAGACTGGGAAGGATGCTGAAAAAAATGTGGATGCTGCTGTG | |
| | ACGCTCTGGCTGAAGAAGGGGACCCCTGCCAGCAAACTGATGCTTGGCATGCCCGCC | |
| | TACGGACGCTCCTTCACCCTGGCCTCCTCATCAGACAGTGGAGTTGGGGCCCCAGCC | |
| | ACAGGACCTGGGGCCCCAGGCCCCTATACTAAGGAAAAGGGGATACTGGTTTACTTT | |
| | GAGGTCTGCTCCTGGAAGGGAAAACAGAGAATCGAGGACCAGAAGGTGCCCTATGTC | |
| | TCCCAGGGCAACCAATGGGTGGGATTTGATGACAGGGAAAGCTTCAGAGCCAAGGCT | |
| | GCCTATGTGAAACAGAAGGGACTAGGAGGGGCCATGGTCTGGATCCTGGATGGGGAT | |
| | GACTTCAAAGGTTCCTTCTGCAACGAGGGCCAGTACCCTCTCATCCGGACACTACAC | |
| | CAGGAGCTGAGCCTTTCATCCGGGCCTCCAAGAAGCCCAGAACAGGAAGTACCTGGG | |
| | CCACACCAGCCTTCTGAGCCCGAGCAGGGCCCCAGCCCAGGGCTAGATAACTTCTGC | |
| | CAAGGCAAAGCTGATGGGCTCTACCCCAACCCTACAGAAAAGTCCAGTTTCTACAGC | |
| | TGTGGAGGGGGGCGACTGTTCCAGCACAGCTGCCCCCCAGGCCTGGTGTTTATCGAC | |
| | TCTTGCAAATGCTGTACCTGGGGTTGA | |
| 34 | AACCACCAGATCAGCACTGTA | Target sequence |
| | | for miRNA in |
| | | rat Chit1 |
| | | sequence. |
| 35 | TGCTGTACAGTGCTGATCTGGTGGTTGTTTTGGCCACTGACTGACAACCACCATCAG | MicroRNA |
| | CACTGTA | sequence |
| | | targeting SEQ |
| | | ID NO: 34 |
| 36 | GCTTTGGTACCCAGAAGTTCA | Target sequence |
| | | for miRNA in |
| | | rat Chit1 |
| | | sequence. |
| 37 | GCCCAGCAGTAGACAAAGAGA | Target sequence |
| | | for miRNA in |
| | | rat Chit1 |
| | | sequence. |
| 38 | TGCTGTCTCTTTGTCTACTGCTGGGCGTTTTGGCCACTGACTGACGCCCAGCAAGAC | MicroRNA |
| | AAAGAGA | sequence |
| | | targeting SEQ |
| | | ID NO: 37 |
| 39 | CTCCTTCTGACTGCAGCTGTA | Target sequence |
| | | for miRNA in |
| | | rat Chit1 |
| | | sequence. |
| 40 | TGCTGTACAGCTGCAGTCAGAAGGAGGTTTTGGCCACTGACTGACCTCCTTCTCTGC | MicroRNA |
| | AGCTGTA | sequence |
| | | targeting SEQ |
| | | ID NO: 39 |
| 41 | GGCCATGTGGATGCTGGTTAT | Target sequence |
| | | for miRNA in |
| | | rat Chit1 |
| | | sequence. |
| 42 | AGATTGCTCAGAGCTTGGATT | Target sequence |
| | | for miRNA in |
| | | rat Chit1 |
| | | sequence. |
| 43 | TGCTGAATCCAAGCTCTGAGCAATCTGTTTTGGCCACTGACTGACAGATTGCTGAGC | MicroRNA |
| | TTGGATT | sequence |
| | | targeting SEQ |
| | | ID NO: 42 |
| 44 | AACCAATGGGTGGGATTTGAT | Target sequence |
| | | for miRNA in |
| | | rat Chit1 |
| | | sequence. |
| 45 | TGGGATTTGATGACAGGGAAA | Target sequence |
| | | for miRNA in |
| | | rat Chit1 |
| | | sequence. |
| 46 | TGCTGTTTCCCTGTCATCAAATCCCAGTTTTGGCCACTGACTGACTGGGATTTTGAC | MicroRNA |
| | AGGGAAA | sequence |
| | | targeting SEQ |
| | | ID NO: 45 |
| 47 | ACTTCAAAGGTTCCTTCTGCA | Target sequence |
| | | for miRNA in |
| | | rat Chit1 |
| | | sequence. |
| 48 | TGGTGTTTATCGACTCTTGC | Target sequence |
| | | for miRNA in |
| | | rat Chit1 |
| | | sequence. |
| 49 | ATGGGCATGAGGGCGGCACTGACAGGCTTTGCGGTCCTGATGCTGCTCCAGAGCTGC | BC005611.2:48- |
| | TCTGCGTACAAGCTGGTCTGCTACTTCACCAGCTGGTCCCAGTACCGGGAAGGCGTT | 1193 Mus |
| | GGAAGCTTCTTACCAGACGCCATCCAACCTTTCCTGTGCACCCACATCATCTACAGC | musculus |
| | TTTGCCAACATCAGCAGCGACAACATGCTTAGCACATGGGAGTGGAATGACGAGTCG | chitinase 3- |
| | AACTATGACAAGCTGAATAAACTGAAGACCAGAAACACCAACCTGAAGACCCTCCTG | like 1, mRNA |
| | TCTGTTGGAGGGTGGAAATTTGGCGAAAAAAGATTTTCCGAGATTGCCTCCAACACT | (CDNA clone |
| | GAGAGACGCACTGCTTTCGTCCGGTCGGTAGCCCCGTTCCTGCGTTCTTATGGCTTT | MGC:7884 |
| | GATGGGCTGGATCTCGCCTGGCTCTACCCTCGCTTAAGAGACAAGCAGTATTTCTCC | IMAGE:3582304), |
| | ACCCTGATCAAGGAACTGAATGCGGAATTCACAAAGGAGGTCCAGCCAGGCAGAGAG | complete cds |
| | AAACTCCTGCTCAGCGCAGCTTTGTCAGCAGGAAAGGTGGCCATTGACACTGGCTAT | |
| | GACATCGCCCAGATAGCCCAACACCTGGATTTTATCAATCTCATGACCTACGATTTC | |
| | CATGGAGTCTGGCGCCAAATCACAGGCCACCACAGCCCCCTCTTCCAAGGCCAGAAG | |
| | GACACTAGGTTTGACAGATACAGCAATGTGAACTATGCCGTGCAGTACATGATACGT | |
| | CTGGGAGCCCAGGCCAGCAAGCTACTGATGGGCATCCCCACCTTTGGGAAGAGCTTC | |
| | ACTCTGGCATCTTCTGAAAATCAGTTGGGAGCTCCAATCTCAGGGGAAGGATTACCA | |
| | GGCCGGTTCACCAAGGAGGCAGGGACCCTGGCCTACTACGAGATATGCGACTTCCTC | |
| | AAAGGAGCTGAAGTACATCGACTCTCCAACGAGAAGGTTCCCTTCGCTACCAAGGGC | |
| | AACCAGTGGGTGGGGTATGAGGACAAGGAGAGTGTCAAAAACAAGGTTGGGTTCCTG | |
| | AAGGAGAAGAAGCTGGCAGGAGCCATGGTGTGGGCACTGGATTTGGATGATTTCCAG | |
| | GGCACCTGTCAGCCGAAGGAATTCTTCCCGCTCACCAACGCCATCAAGGATGCCCTG | |
| | GCTTAG | |
| 50 | CTTTCCTGTGCACCCACATCA | Target sequence |
| | | for miRNA in |
| | | mouse CHI3L1 |
| | | sequence. |
| 51 | TGCTGTGATGTGGGTGCACAGGAAAGGTTTTGGCCACTGACTGACCTTTCCTGCACC | MicroRNA |
| | CACATCA | sequence |
| | | targeting SEQ |
| | | ID NO: 50 |
| 52 | CGAACTATGACAAGCTGAATA | Target sequence |
| | | for miRNA in |
| | | mouse CHI3L1 |
| | | sequence |
| 53 | CACTGAGAGACGCACTGCTTT | Target sequence |
| | | for miRNA in |
| | | mouse CHI3L1 |
| | | sequence |
| 54 | GCTTAAGAGACAAGCAGTATT | Target sequence |
| | | for miRNA in |
| | | mouse CHI3L1 |
| | | sequence |
| 55 | GAACTGAATGCGGAATTCACA | Target sequence |
| | | for miRNA in |
| | | mouse CHI3L1 |
| | | sequence. |
| 56 | TGCTGTGTGAATTCCGCATTCAGTTCGTTTTGGCCACTGACTGACGAACTGAACGGA | MicroRNA |
| | ATTCACA | sequence |
| | | targeting SEQ |
| | | ID NO: 55 |
| 57 | GCAGCTTTGTCAGCAGGAAAG | Target sequence |
| | | for miRNA in |
| | | mouse CHI3L1 |
| | | sequence |
| 58 | CTAGGTTTGACAGATACAGCA | Target sequence |
| | | for miRNA in |
| | | mouse CHI3L1 |
| | | sequence |
| 59 | TTCCTCAAAGGAGCTGAAGTA | Target sequence |
| | | for miRNA in |
| | | mouse CHI3L1 |
| | | sequence. |
| 60 | TGCTGTACTTCAGCTCCTTTGAGGAAGTTTTGGCCACTGACTGACTTCCTCAAGAGC | MicroRNA |
| | TGAAGTA | sequence |
| | | targeting SEQ |
| | | ID NO: 59 (si5- |
| | | rM907-F) |
| 61 | GGCACTGGATTTGGATGATTT | Target sequence |
| | | for miRNA in |
| | | mouse CHI3L1 |
| | | sequence. |
| 62 | TGCTGAAATCATCCAAATCCAGTGCCGTTTTGGCCACTGACTGACGGCACTGGTTGG | MicroRNA |
| | ATGATTT | sequence |
| | | targeting SEQ |
| | | ID NO: 61 |
| 63 | CGAAGGAATTCTTCCCGCTCA | Target sequence |
| | | for miRNA in |
| | | mouse CHI3L1 |
| | | sequence |
| 64 | ATGCTGCTCCAGAGCTGCTCTGCGTACAAACTGGTCTGCTACTACACCAACTGGTCC | BC091365.1:96- |
| | CAGTACCGGGAAGGCAATGGGAGCTGCTTCCCAGATGCCCTCGACCATTCCCTGTGC | 1208 Rattus |
| | ACCCATATCATCTACAGCTTTGCCAACATCAGCAACAACAAGCTCAGCACATCGGAG | norvegicus |
| | TGGAATGACGTAACCCTGTATGGCATGCTGAATACTCTCAAGACCAGAAACCCCAGA | chitinase 3- |
| | CTGAAGACACTGCTGTCTGTTGGAGGATGGAGCTTTGGCTCAGAAAGATTTTCCAGG | like 1, mRNA |
| | ATTGTCTCCAACGCTAAGAGTCGCAAGACTTTCGTCCAGTCGGTAGCTCCCTTCCTG | (cDNA clone |
| | CGGACCTATGGCTTTGATGGACTGGATCTCGCCTGGCTCTACCCGGGCCCGAAAGAC | MGC:109420 |
| | AAGCAACATTTTACCACACTGATCAAGGAACTGAAGGCGGAATTCACAAAGGAAGTC | IMAGE:7319171), |
| | CAGCCAGGCACAGAGAAACTCCTGCTCAGTGCTGCCGTGTCAGCAGGAAAGGTGACC | complete cds |
| | CTTGACAGTGGCTATGATGTTGCCCAGATAGCCCAACACCTAGATTTCATTAATCTC | |
| | ATGACCTATGATTTCCATGGAACCTGGCGCCACACCACAGGACATCACAGCCCCCTC | |
| | TTCCGAGGCCAGCAGGACACTGGGCCTGACAGATTCAGCAATGTGGACTATGGTGTG | |
| | GGGTACATGCTAAGGCTGGGAGCCCCCACCAACAAGCTAGTGATGGGTATCCCCACC | |
| | TTTGGAAAGAGCTTCACTCTGGCATCTTCTGAGAATCAAGTGGGAGCTCCAATCACA | |
| | GGGTCAGGATTACCAGGCCGCTACACCAAGGAGAAAGGGACCCTCGCCTACTACGAG | |
| | ATATGCGACTTCCTCAGAGGAGCTGAAGTACATAGAATTCTTGGCCAGCAGGTTCCC | |
| | TTTGCTACCAAGGGCAACCAGTGGGTGGGGTATGATGACCCGGAGAGCGTCAAAAAC | |
| | AAGGTGAAGTACCTGAAGAACAAGCAGCTGGCAGGAGCCATGGTGTGGGCAGTGGAT | |
| | TTGGATGATTTCCGGGGCTCCTTCTGTGGGCATAACGTACACTTCCCGCTCACCAAC | |
| | GCCATCAAGGAGGCCCTGGCTGTGGCTTAG | |
| 65 | CTTTCCTGTGCACCCACATCA | Target sequence |
| | | for miRNA in |
| | | Rat CHI3L1 |
| | | sequence. |
| 66 | TGCTGTGATGTGGGTGCACAGGAAAGGTTTTGGCCACTGACTGACCTTTCCTGCACC | MicroRNA |
| | CACATCA | sequence |
| | | targeting SEQ |
| | | ID NO: 65 |
| 67 | CGAACTATGACAAGCTGAAT | Target sequence |
| | | for miRNA in |
| | | Rat CHI3L1 |
| | | sequence |
| 68 | CACTGAGAGACGCACTGCTTT | Target sequence |
| | | for miRNA in |
| | | Rat CHI3L1 |
| | | sequence |
| 69 | GCTTAAGAGACAAGCAGTATT | Target sequence |
| | | for miRNA in |
| | | Rat CHI3L1 |
| | | sequence |
| 70 | GAACTGAATGCGGAATTCACA | Target sequence |
| | | for miRNA in |
| | | Rat CHI3L1 |
| | | sequence. |
| 71 | TGCTGTGTGAATTCCGCATTCAGTTCGTTTTGGCCACTGACTGACGAACTGAACGGA | MicroRNA |
| | ATTCACA | sequence |
| | | targeting SEQ |
| | | ID NO: 70 |
| 72 | GCAGCTTTGTCAGCAGGAAAG | Target sequence |
| | | for miRNA in |
| | | Rat CHI3L1 |
| | | sequence |
| 73 | CTAGGTTTGACAGATACAGCA | Target sequence |
| | | for miRNA in |
| | | Rat CHI3L1 |
| | | sequence |
| 74 | TTCCTCAAAGGAGCTGAAGTA | Target sequence |
| | | for miRNA in |
| | | Rat CHI3L1 |
| | | sequence. |
| 75 | TGCTGTACTTCAGCTCCTTTGAGGAAGTTTTGGCCACTGACTGACTTCCTCAAGAGC | MicroRNA |
| | TGAAGTA | sequence |
| | | targeting SEQ |
| | | ID NO: 74 |
| 76 | GGCACTGGATTTGGATGATTT | Target sequence |
| | | for miRNA in |
| | | Rat CHI3L1 |
| | | sequence. |
| 77 | TGCTGAAATCATCCAAATCCAGTGCCGTTTTGGCCACTGACTGACGGCACTGGTTGG | MicroRNA |
| | ATGATTT | sequence |
| | | targeting SEQ |
| | | ID NO: 76 |
| 78 | CGAAGGAATTCTTCCCGCTCA | Target sequence |
| | | for miRNA in |
| | | Rat CHI3L1 |
| | | sequence. |
| 79 | TGCTGAGAGTATTCAGCATGCCATACGTTTTGGCCACTGACTGACGTATGGCACTGA | si9-R189-F |
| | ATACTCT | |
| 80 | TGCTGCTCAGAAGATGCCAGAGTGAAGTTTTGGCCACTGACTGACTTCACTCTCATC | si11-mR754-F |
| | TTCTGAG | |
| 81 | TGCTGAAATCATCCAAATCCACTGCCGTTTTGGCCACTGACTGACGGCAGTGGTTGG | si13-mR1017-F |
| | ATGATTT | |
| 82 | TGCTGTAGATCAGGTGAGTACACAGGGTTTTGGCCACTGACTGACCCTGTGTACACC | si29-M147-F |
| | TGATCTA | |
| 83 | CAGCGGCCGCATGCTGCTCCAGAGCTGC | 199-rCHI3L1-F |
| 84 | CAGCGGCCGCATGGTGCAGTCCCTGGCCTT | 201-rChit1-F |
| 85 | CAGCGGCCGCATGGGCATGAGGGCGGCAC | 203-mBRP-39-F |
| 86 | CAGCGGCCGCATGGCCAAGCTCATTCTTGTC | 207-mYm-1-F |
| 87 | tgcg cgctcgctcg ctcactgagg ccgcccgggc aaagcccggg | rAAV vector |
| | cgtcgggcga cctttggtcg cccggcctca gtgagcgage gagcgcgcag | |
| | agagggagtg gaattcacgc gtggatctga attcaattca cgcgtggtac | |
| | ctctggtcgt tacataactt acggtaaatg gcccgcctgg ctgaccgccc | |
| | aacgaccccc gcccattgac gtcaataatg acgtatgttc ccatagtaac | |
| | gccaataggg actttccatt gacgtcaatg ggtggagtat ttacggtaaa | |
| | ctgcccactt ggcagtacat caagtgtatc atatgccaag tacgccccct | |
| | attgacgtca atgacggtaa atggcccgcc tggcattatg cccagtacat | |
| | gaccttatgg gactttccta cttggcagta catctactcg aggccacgtt | |
| | ctgcttcact ctccccatct cccccccctc cccaccccca attttgtatt | |
| | tatttatttt ttaattattt tgtgcagcga tgggggcggg gggggggggg | |
| | gggcgcgcgc caggcggggc ggggcggggc gaggggcggg gcggggcgag | |
| | gcggagaggt gcggcggcag ccaatcagag cggcgcgctc cgaaagtttc | |
| | cttttatggc gaggcggcgg cggcggcggc cctataaaaa gcgaagcgcg | |
| | cggcgggcgg gagcgggatc agccaccgcg gtggcggcct agagtcgacg | |
| | aggaactgaa aaaccagaaa gttaactggt aagtttagtc tttttgtctt | |
| | ttatttcagg tcccggatcc ggtggtggtg caaatcaaag aactgctcct | |
| | cagtggatgt tgcctttact tctaggcctg tacggaagtg ttacttctgc | |
| | tctaaaagct gcggaattgt acccgcggcc gatccaccgg tcgccaccat | |
| | ggtgagcaag ggcgaggagc tgttcaccgg ggtggtgccc atcctggtcg | |
| | agctggacgg cgacgtaaac ggccacaagt tcagcgtgtc cggcgagggc | |
| | gagggcgatg ccacctacgg caagctgacc ctgaagttca tctgcaccac | |
| | cggcaagctg cccgtgccct ggcccaccct cgtgaccacc ctgacctacg | |
| | gcgtgcagtg cttcagccgc taccccgacc acatgaagca gcacgacttc | |
| | ttcaagtccg ccatgcccga aggctacgtc caggagcgca ccatcttctt | |
| | caaggacgac ggcaactaca agacccgcgc cgaggtgaag ttcgagggcg | |
| | acaccctggt gaaccgcatc gagctgaagg gcatcgactt caaggaggac | |
| | ggcaacatcc tggggcacaa gctggagtac aactacaaca gccacaacgt | |
| | ctatatcatg gccgacaagc agaagaacgg catcaaggtg aacttcaaga | |
| | tccgccacaa catcgaggac ggcagcgtgc agctcgccga ccactaccag | |
| | cagaacaccc ccatcggcga cggccccgtg ctgctgcccg acaaccacta | |
| | cctgagcacc cagtccgccc tgagcaaaga ccccaacgag aagcgcgatc | |
| | acatggtcct gctggagttc gtgaccgccg ccgggatcac tctcggcatg | |
| | gacgagctgt acaagtaaag cggccctagc gtttccggcg acggtgctag | |
| | cgtcgaccag tggatcctgg aggcttgctg aaggctgtaT caggacacaa | |
| | ggcctgttac tagcactcac atggaacaaa tggcccagat ctggccgc | |
| | CAGGACACAA GGCCTGTTAC TAGCACTCAC ATGGAACAAA TGGCCCAGAT | |
| | CTGGCCGCAC TCGAAAACGG GCCCTCTAGA CTCGAGGACG GGGTGAACTA | |
| | CGCCTGAGGA TCCGATCTTT TTCCCTCTGC CAAAAATTAT GGGGACATCA | |
| | TGAAGCCCCT TGAGCATCTG ACTTCTGGCT AATAAAGGAA ATTTATTTTC | |
| | ATTGCAATAG TGTGTTGGAA TTTTTTGTGT CTCTCACTCG GAAGCAATTC | |
| | GTTGATCTGA ATTTCGACCA CCCATAATAC CCATTACCCT GGTAGATAAG | |
| | TAGCATGGCG GGTTAATCAT TAACTACAAG GAACCCCTAG TGATGGAGTT | |
| | GGCCACTCCC TCTCTGCGCG CTCGCTCGCT CACTGAGGCC GGGCGACCAA | |
| | AGGTCGCCCG ACGCCCGGGC TTTGCCCGGG CGGCCTCAGT GAGCGAGCGA | |
| | GCGCG | |
| 88 | GTTACATAACTT ACGGTAAATG GCCCGCCTGG CTGACCGCCC AACGACCCCC | Chimeric CMV- |
| | GCCCATTGAC GTCAATAATG ACGTATGTTC CCATAGTAAC GCCAATAGGG | chicken ß-actin |
| | ACTTTCCATT GACGTCAATG GGTGGAGTAT TTACGGTAAA CTGCCCACTT | promoter (CBA) |
| | GGCAGTACAT CAAGTGTATC ATATGCCAAG TACGCCCCCT ATTGACGTCA | |
| | ATGACGGTAA ATGGCCCGCC TGGCATTATG CCCAGTACAT GACCTTATGG | |
| | GACTTTCCTA CTTGGCAGTA CATCTACTCG AGGCCACGTT CTGCTTCACT | |
| | CTCCCCATCT CCCCCCCCTC CCCACCCCCA ATTTTGTATT TATTTATTTT | |
| | TTAATTATTT TGTGCAGCGA TGGGGGCGGG GGGGGGGGGG GGGCGCGCGC | |
| | CAGGCGGGGC GGGGCGGGGC GAGGGGCGGG GCGGGGCGAG GCGGAGAGGT | |
| | GCGGCGGCAG CCAATCAGAG CGGCGCGCTC CGAAAGTTTC CTTTTATGGC | |
| | GAGGCGGCGG CGGCGGCGGC CCTATAAAAA GCGAAGCGCG CGGCGGGCGG | |
| | GAGCGGGATC AGCCACCGCG GTGGCGGCCT AGAGTCGACG AGGAACTGAA | |
| | AAACCAGAAA GTTAACTGGT AAGTTTAGTC TTTTTGTCTT TTATTTCAGG | |
| | TCCCGGATCC GGTGGTGGTG CAAATCAAAG AACTGCTCCT CAGTGGATGT | |
| | TGCCTTTACT TCTAG | |

What is claimed is:

1. An engineered vector-mediated system for modifying the expression of a chitinase protein in a target cell, the system comprising:
a. a nucleic acid expression construct comprising:
i. a promoter operably linked to a nucleic acid sequence encoding a programmable nucleic acid modification system targeted to a nucleotide sequence encoding the chitinase protein; or
ii. a nucleotide sequence encoding the chitinase protein operably linked to a promoter; and
b. a nucleic acid delivery vector comprising the nucleic acid expression construct for delivering the nucleic acid expression construct to the target cell;
wherein expressing the programmable nucleic acid modification system or chitinase protein modifies the expression of the chitinase protein.

2. The vector-mediated system of claim 1, wherein the chitinase protein is an SI-CLP protein, a chitinase-3 like-protein-2 (CHI3L2; YKL-39) protein, a CHI3L1 (YKL-40) protein, a chitriosidase (Chit-1) protein, an AMCase protein, an oviductin protein, a YM1 protein, a YM2 protein, or any combination thereof.

3. The vector-mediated system of claim 2, wherein the YM1 protein comprises an amino acid sequence encoded by a nucleotide sequence comprising at least about 75% or more, at least about 85% or more, at least about 95% or more, or 100% sequence identity with the nucleic acid sequence of SEQ ID NO: 2.

4. The vector-mediated system of claim 3, wherein the programmable nucleic acid modification system is an miRNA molecule comprising a nucleotide sequence complementary to a target sequence within the nucleotide sequence encoding the YM1 protein.

5. The vector-mediated system of claim 4, wherein the miRNA molecule comprises a nucleotide sequence selected from SEQ ID NOs: 4, 6, 11, 14, 87, and any combination thereof.

6. The vector-mediated system of claim 4, wherein the target sequence within a gene encoding the YM1 protein is selected from SEQ ID NOs: 3, 5, 7-10, 12, 13, and any combination thereof.

7. The vector-mediated system of claim 2, wherein the Chit-1 protein comprises an amino acid sequence encoded by a nucleotide sequence comprising at least about 75% or more, at least about 85% or more, at least about 95% or more, or 100% sequence identity with the nucleic acid sequence of SEQ ID NO: 17.

8. The vector-mediated system of claim 7, wherein the programmable nucleic acid modification system is an miRNA molecule comprising a nucleotide sequence complementary to a target sequence within the nucleotide sequence encoding the Chit-1 protein.

9. The vector-mediated system of claim 8, wherein the miRNA molecule comprises a nucleotide sequence selected from SEQ ID NOs: 20, 23, 25, 29, 31, and any combination thereof.

10. The vector-mediated system of claim 8, wherein the target sequence within a gene encoding the Chit-1 protein is selected from SEQ ID NOs: 18, 19, 21, 22, 24, 26-28, 30, 32, and any combination thereof.

11. The vector-mediated system of claim 2, wherein the Chit-1 protein comprises an amino acid sequence encoded by a nucleotide sequence comprising at least about 75% or more, at least about 85% or more, at least about 95% or more, or 100% sequence identity with the nucleic acid sequence of SEQ ID NO: 33.

12. The vector-mediated system of claim 11, wherein the programmable nucleic acid modification system is an miRNA molecule comprising a nucleotide sequence complementary to a target sequence within the nucleotide sequence encoding the Chit-1 protein.

13. The vector-mediated system of claim 12, wherein the miRNA molecule comprises a nucleotide sequence selected from SEQ ID NOs: 35, 38, 40, 43, 46, and any combination thereof.

14. The vector-mediated system of claim 12, wherein the target sequence within a gene encoding the Chit-1 protein is selected from SEQ ID NOs: 34, 36, 37, 39, 41, 42, 44, 45, 47, 48, and any combination thereof.

15. The vector-mediated system of claim 2, wherein the CHI3L1 protein comprises an amino acid sequence encoded by a nucleotide sequence comprising at least about 75% or more, at least about 85% or more, at least about 95% or more, or 100% sequence identity with the nucleic acid sequence of SEQ ID NO: 49.

16. The vector-mediated system of claim 15, wherein the programmable nucleic acid modification system is an miRNA molecule comprising a nucleotide sequence complementary to a target sequence within the nucleotide sequence encoding the CHI3L1 protein.

17. The vector-mediated system of claim 16, wherein the miRNA molecule comprises a nucleotide sequence selected from SEQ ID NOs: 51, 56, 60, 62, and any combination thereof.

18. The vector-mediated system of claim 16, wherein the target sequence within a gene encoding the CHI3L1 protein is selected from SEQ ID NOs: 50, 52-55, 57-59, 61, 63, and any combination thereof.

19. The vector-mediated system of claim 2, wherein the CHI3L1 protein comprises an amino acid sequence encoded by a nucleotide sequence comprising at least about 75% or more, at least about 85% or more, at least about 95% or more, or 100% sequence identity with the nucleic acid sequence of SEQ ID NO: 64.

20. The vector-mediated system of claim 19, wherein the programmable nucleic acid modification system is an miRNA molecule comprising a nucleotide sequence complementary to a target sequence within the nucleotide sequence encoding the CHI3L1 protein.

21. The vector-mediated system of claim 20, wherein the miRNA molecule comprises a nucleotide sequence selected from SEQ ID NOs: 66, 71, 75, 77, and any combination thereof.

22. The vector-mediated system of claim 20, wherein the target sequence within a gene encoding the CHI3L1 protein is selected from SEQ ID NOs: 65, 67-70, 72-74, 76, 78, and any combination thereof.

23. The vector-mediated system of claim 1, wherein the promoter is a chimeric CMV-chicken β-actin promoter (CBA) promoter comprising a nucleic acid sequence comprising at least about 75% or more, at least about 85% or more, at least about 95% or more, or 100% sequence identity with a sequence selected from SEQ ID NO: 87.

24. The vector-mediated system of claim 1, wherein the programmable nucleic acid modification system is an interfering nucleic acid molecule.

25. The vector-mediated system of claim 24, wherein the interfering nucleic acid molecule is selected from the group consisting of an antisense molecule, siRNA molecules, single-stranded siRNA molecules, miRNA molecules, piRNA molecules, lncRNA molecules, shRNA molecules, and any combination thereof.

26. The vector-mediated system of claim 25, wherein the interfering nucleic acid molecule is an miRNA molecule.

27. The vector-mediated system of claim 1, wherein the nucleic acid delivery vector is a recombinant AAV (rAAV) vector comprising the nucleic acid expression construct inserted between the inverted terminal repeats (ITR) of an AAV virus genome.

28. The vector-mediated system of claim 27, wherein the rAAV vector comprises a nucleotide sequence comprising at least about 75% or more, at least about 85% or more, at least about 95% or more, or 100% sequence identity with the nucleotide sequence of SEQ ID NO: 88.

29. The vector-mediated system of claim 1, wherein the programmable nucleic acid modification system is an RNA-guided clustered regularly interspersed short palindromic repeats (CRISPR)/CRISPR-associated (Cas) (CRISPR/Cas) nuclease system, a CRISPR/Cpf1 nuclease system, a zinc finger nuclease (ZFN), a transcription activator-like effector nuclease (TALEN), a meganuclease, a ribozyme, or a programmable DNA binding domain linked to a nuclease domain.

30. The vector-mediated system of claim 29, wherein the programmable nucleic acid modification system is a CRISPR/Cas tool modified for transcriptional regulation of a locus.

31. The vector-mediated system of any one of the preceding claims, wherein the target cell or tissue type is a cell or tissue type wherein expression of the chitinase protein is associated with a disease condition.

32. The vector-mediated system of any preceding claim, wherein the target cell or tissue type is an organ in the body, a cell in the nervous system, a cancer cell or tumor, or a cell of the immune system.

33. The vector-mediated system of any preceding claim, wherein decreasing the expression of the chitinase protein decreases the inflammatory profile of the one or more chitinase genes in distinct glial subsets.

34. The vector-mediated system of any preceding claim, wherein the target cell or tissue type is in a subject having ALS, PD, or MSA, and wherein the expression of CHI3L1 is increased, wherein the expression of CHI3L2 is increased, the expression of Chit-1 is decreased, or any combination thereof.

35. A recombinant AAV (rAAV) vector-mediated system for modifying the expression of a chitinase protein in a target cell, the rAAV vector comprising a nucleic acid expression construct inserted between the ITRs of an AAV virus genome, the nucleic acid expression construct comprising:
a. a nucleic acid sequence encoding a programmable nucleic acid modification system targeted to a nucleotide sequence encoding a chitinase protein operably linked to a promoter; or
b. a nucleotide sequence encoding a chitinase protein operably linked to a promoter;
wherein expressing the programmable nucleic acid modification system or chitinase protein modifies the expression of the chitinase protein.

36. An AAV virion comprising an AAV capsid protein encapsidating a recombinant rAAV vector comprising a nucleic acid expression construct inserted between the ITRs of an AAV virus genome, the nucleic acid expression construct comprising:
a. a nucleic acid sequence encoding a programmable nucleic acid modification system targeted to a nucleotide sequence encoding a chitinase protein operably linked to a promoter; or
b. a nucleotide sequence encoding a chitinase protein operably linked to a promoter;
wherein expressing the programmable nucleic acid modification system or chitinase protein modifies the expression of the chitinase protein.

37. One or more nucleic acid constructs encoding the engineered vector-mediated system of any of claims 1-34, the rAAV vector of claim 35.

38. A cell comprising the engineered vector-mediated system of any of claims 1-34, the rAAV vector of claim 35, the AAV virion of claim 36, or the one or more nucleic acid constructs of claim 37.

39. A method of treating a disease condition associated with expression of a chitinase protein in a cell or tissue type in a subject in need thereof, the method comprising modifying the expression of one or more chitinase proteins in the cell or tissue type in the subject by administering to the subject a therapeutically effective amount of a composition comprising the engineered vector-mediated system of any of any of claims 1-34, the rAAV vector of claim 35, the AAV virion of claim 36, or the one or more nucleic acid constructs of claim 37.

40. The method of claim 39, wherein the disease condition is a condition associated with inflammation.

41. The method of claim 39, wherein the disease condition is a neurological condition.

42. The method of claim 39, wherein the disease condition is a cancer.

43. A method of treating a neurological condition associated with expression of a chitinase protein in a cell or tissue type in the nervous system in a subject in need thereof, the method comprising modifying the expression of one or more chitinase proteins in the cell or tissue type in the nervous system of the subject by administering to the subject a therapeutically effective amount of a composition comprising the engineered vector-mediated system of any of any of claims 1-34, the rAAV vector of claim 35, the AAV virion of claim 36, or the one or more nucleic acid constructs of claim 37.

44. The method of claim 43, wherein the neurological condition is Alzheimer's disease (AD), Amyotrophic lateral sclerosis (ALS), Parkinson's disease (PD), multiple sclerosis (MS), multiple system atrophy (MSA), ataxia, Bell's palsy, or epilepsy.

45. The method of claim 44, wherein the neurological condition is a synucleinopathy.

46. The method of claim 43, wherein the protein expression modification system reduces the expression of the one or more chitinase proteins to decrease the inflammatory profile in distinct glial subsets.

47. The method of claim 43, wherein the one or more chitinase proteins are Chit-1, CHI3L1, CHI3L2, or any combination thereof.

48. The method of claim 43, wherein the neurological condition is ALS.

49. The method of claim 43, wherein the cell or tissue type is an activated glial subtype.

50. The method of claim 43, wherein the cell or tissue type is an activated astrocyte.

51. The method of claim 43, wherein the protein expression modification system reduces the expression of CHI3L1 protein in activated astrocytes.

52. A method of treating amyotrophic lateral sclerosis (ALS) in a subject in need thereof, the method comprising decreasing the expression of one or more chitinase proteins in the nervous system of the subject by administering to the subject a therapeutically effective amount of a composition comprising the engineered vector-mediated system of any of any of claims 1-34, the rAAV vector of claim 35, the AAV virion of claim 36, or the one or more nucleic acid constructs of claim 37.

53. The method of claim 52, wherein the protein expression modification system modifies the expression of one or more chitinase proteins in activated glial subtypes.

54. The method of claim 52, wherein the protein expression modification system modifies the expression of Chit-1, CHI3L1, CHI3L2, or any combination thereof in the central nervous system.

55. The method of claim 52, wherein the protein expression modification system modifies the expression of Chit-1, CHI3L1, CHI3L2, or any combination thereof in a cell of glial lineage.

56. The method of claim 52, wherein the protein expression modification system reduces the expression of Chit-1 in activated microglia, reduces the expression of CHI3L1 in activated astrocytes, reduces the expression of CHI3L2 in activated microglia, or any combination thereof.

57. A method of treating Parkinson's disease (PD) in a subject in need thereof, the method comprising reducing the expression of one or more chitinase proteins in a cell or tissue type in the nervous system by administering to the subject a therapeutically effective amount of a composition comprising the engineered vector-mediated system of any of any of claims 1-34, the rAAV vector of claim 35, the AAV virion of claim 36, or the one or more nucleic acid constructs of claim 37.

58. The method of claim 57, wherein the protein expression modification system modifies the expression of one or more chitinase proteins in activated astrocytes.

59. The method of claim 57, wherein the protein expression modification system reduces the expression of CHI3L1 proteins in activated astrocytes.

60. A method of treating an MSA disease in a subject in need thereof, the method comprising reducing the expression of one or more chitinase proteins in a cell or tissue type in the nervous system by administering to the subject a therapeutically effective amount of a composition comprising the engineered vector-mediated system of any of any of claims 1-34, the rAAV vector of claim 35, the AAV virion of claim 36, or the one or more nucleic acid constructs of claim 37.

61. The method of claim 60, wherein the protein expression modification system modifies the expression of one or more chitinase proteins in activated astrocytes.

62. The method of claim 60, wherein the protein expression modification system reduces the expression of CHI3L1 proteins in activated astrocytes.

63. Use of one or more engineered of a composition comprising the engineered vector-mediated system of any of any of claims 1-34, the rAAV vector of claim 35, the AAV virion of claim 36, or the one or more nucleic acid constructs of claim 37, for the treatment or prevention of a neuronal condition in a subject in need thereof.

64. A kit for modifying the expression of a chitinase protein in a target cell, the kit comprising one or more vector-mediated engineered systems of a composition comprising the engineered vector-mediated system of any of any of claims 1-34, the rAAV vector of claim 35, the AAV virion of claim 36, or the one or more nucleic acid constructs of claim 37, for the treatment or prevention of a neuronal condition in a subject in need thereof.
